SEQUENCE LISTING (8 sequences)

SEQ ID NO: 1 - DNA, 20 bp, synthetic construct
  source 1..20: mol_type = other DNA
aatgatacggcgaccaccga

SEQ ID NO: 2 - DNA, 21 bp, synthetic construct
  source 1..21: mol_type = other DNA
caagcagaagacggcatacga

SEQ ID NO: 3 - DNA, 30 bp, synthetic construct
  source 1..30: mol_type = other DNA
ttttttttttaatgatacggcgaccaccga

SEQ ID NO: 4 - DNA, 31 bp, synthetic construct
  source 1..31: mol_type = other DNA
ttttttttttcaagcagaagacggcatacga

SEQ ID NO: 5 - DNA, 33 bp, synthetic construct
  source 1..33: mol_type = other DNA
acactctttccctacacgacgctcttccgatct

SEQ ID NO: 6 - DNA, 20 bp, synthetic construct
  source 1..20: mol_type = other DNA
acactctttccctacacgac

SEQ ID NO: 7 - DNA, 34 bp, synthetic construct
  source 1..34: mol_type = other DNA
gtgactggagttcagacgtgtgctcttccgatct

SEQ ID NO: 8 - DNA, 21 bp, synthetic construct
  source 1..21: mol_type = other DNA
gtgactggagttcagacgtgt

(19) United States
(12) Patent Application Publication
(10) Pub. No.: US 2025/0101493 A1
(43) Pub. Date: Mar. 27, 2025

(54) SPATIAL OMICS PLATFORMS AND SYSTEMS

(71) Applicant: Illumina, Inc., San Diego, CA (US)

(72) Inventors: Andrea MANZO, San Diego, CA (US); Aathavan KARUNAKARAN, San Diego, CA (US); Jeffrey S. FISHER, San Diego, CA (US); Jeffrey G. MANDELL, San Diego, CA (US); Eric Hans VERMAAS, San Diego, CA (US); Fiona KAPER, San Diego, CA (US); Adam WHITE, San Diego, CA (US); Andrew J. PRICE, San Diego, CA (US); Andrew Zachary OSTROW, San Diego, CA (US); Jeffrey BRODIN, San Diego, CA (US); Sarah Munchel SHULTZABERGER, San Diego, CA (US)

(21) Appl. No.: 18/725,576

(22) PCT Filed: Dec. 29, 2022
(86) PCT No.: PCT/US2022/082546

§ 371 Date: Jun. 28, 2024

Related U.S. Application Data
(60) Provisional application No. 63/295,831, filed on Dec. 31, 2021.

(51) Int. Cl. C12Q 1/6806; C12N 15/10; C12Q 1/6804

(52) U.S. Cl. CPC C12Q 1/6806; C12N 15/1065; C12N 15/1082; C12N 15/1096; C12Q 1/6804

(57) ABSTRACT

The disclosure provides compositions, methods, and kits that facilitate the characterization of omic variation in tissues while preserving spatial information related to the origin of target analytes in the tissue.

### CROSS REFERENCE TO RELATED APPLICATIONS

This application claim priority under 35 U.S.C. § 119(e) to U.S. Provisional Application No. 63/295,831, filed Dec. 31, 2021, the disclosure of which is incorporated herein by reference for all purposes.

### FIELD OF THE INVENTION

The disclosure provides compositions, methods, and kits that facilitate the characterization of omic variation in tissues while preserving spatial information related to the origin of target analytes in the tissue.

### INCORPORATION BY REFERENCE OF SEQUENCE LISTING

Accompanying this filing is a Sequence Listing entitled, “00140-028WO1.xml” created on Dec. 29, 2022 and having 8,140 bytes of data, machine formatted on IBM-PC, MS-Windows operating system. The sequence listing is hereby incorporated by reference in its entirety for all purposes.

### BACKGROUND

Existing techniques for the detection and analysis of nucleic acids (e.g., mRNA or genomic DNA) in a tissue sample typically provide spatial or localized information for one or limited number of genes at a time or provide information for all genes in the sample without the desired positional information. Recent interest has focused on the development of techniques that allow the characterization of transcriptomes and/or genomic variations in tissues while preserving spatial information about the tissue. There is a need for methods of characterizing nucleic acids in the context of a tissue sample.

### SUMMARY

The disclosure provides compositions, methods, and kits that facilitate the characterization of omic variation in tissues while preserving spatial information related to the origin of target analytes in the tissue.

In a particular embodiment, the disclosure provides a spatial genomics Assay for Transposase-Accessible Chromatin with high-throughput sequencing (ATAC-Seq) method, comprising: (A) loading nanoparticles into nanowells of an array, wherein the nanoparticles comprise oligos that attached to the nanoparticles through a selectively cleavable linker (e.g., desthiobiotin molecules (ddBio), or PC Linker), the oligos comprising an adapter sequence (e.g., a P5 adapter sequence (SEQ ID NO:1), a P7 adapter sequence (SEQ ID NO:2), etc.), a spatial address sequence, and a transposome hybridization region; (B) decoding the spatial address sequences of the oligos to determine the x,y position of the nanoparticles; (C) placing tissue on top of the nanoparticles in the array, and lysing the cell membranes to access chromatin regions in the tissue; (D) tagmentating the chromatin regions with a transposome complex to form tagmented fragments, and removing the transposome complex; (E) permeabilizing the tissue to allow diffusion of the tagmented fragments to the nanoparticles in the array; (F) capturing the tagmented fragments to the nanoparticles by hybridizing the tagmented fragments to the transposome hybridization region of the oligos; (G) processing the captured tagmented fragments to make nanoparticle bound genomic library constructs for sequencing; (H) releasing the genomic library constructs from the nanoparticles by cleaving the selectively cleavable linker (e.g., using heat and added biotin); (I) obtaining spatial genomics information of the tissue by sequencing the genomic library constructs using a sequencer and mapping the sequencing reads with x,y positions of the nanoparticles. In a further embodiment, the nanoparticles are beads. In another embodiment or a further embodiment, the transposome complex is a TN5 transposome complex. In another embodiment or a further embodiment, the spatial address sequences are decoded by using a decoding-by-hybridization method. In another embodiment or a further embodiment, the cell membranes are lysed by using a mild detergent (e.g., NP40, Tween 20, Bile Salts, Triton X100, and (3-((3-cholamidopropyl)dimethylammonio)-1-propanesulfonate) (CHAPS)). In another embodiment or a further embodiment, the tissue is permeabilized by using a detergent (e.g., Tween 20 and/or Triton X100) and Proteinase K. In another embodiment or a further embodiment, the captured tagmented fragments are processed by using top strand ligation, hybridization to Y-shaped adaptors, and gap-filling ligations steps (e.g., use of a DNA polymerase lacking strand-displacement activity, dNTPs, and a DNA ligase). In another embodiment or a further embodiment, prior to sequencing the library constructs are amplified using PCR. In another embodiment or a further embodiment, the sequencer utilizes sequencing by synthesis technology. In another embodiment or a further embodiment, the hybridization region of the transposome complex is initially blocked by use of an accessory oligo that is then removed in step (E).

In a certain embodiment, the disclosure also provides a spatial multi-omics Assay for Transposase-Accessible Chromatin with high-throughput sequencing (ATAC-Seq) and RNA-Seq method, comprising: (A) loading nanoparticles into nanowells of an array, wherein the nanoparticles comprise two sets of oligos that attached to the nanoparticles through a selectively cleavable linker (e.g., desthiobiotin molecules (ddBio), or PC Linker), the first set of oligos comprising a first adapter sequence (e.g., a P5 adapter sequence (SEQ ID NO:1), a P7 adapter sequence (SEQ ID NO:2), etc.), a spatial address sequence, and a transposome hybridization region, the second set of oligos comprising a second adapter sequence (e.g., a P5 adapter sequence (SEQ ID NO:1), a P7 adapter sequence (SEQ ID NO:2), etc.), a sequencing primer site sequence (e.g., a R1 sequencing primer site sequence (e.g., SEQ ID NO:5-6), a R2 sequencing primer site sequence (e.g., SEQ ID NO:7-8), etc.), a spatial address sequence, a unique molecular identifier (UMI) sequence, and an oligo(dT) sequence; (B) decoding the spatial address sequences of the oligos to determine the x,y position of the nanoparticles; (C) placing tissue on top of the nanoparticles in the array, and lysing the cell membranes to access poly-A RNA transcripts and chromatin regions in the tissue; (D) tagmentating the chromatin regions with a first transposome complex to form tagmented fragments, and removing the first transposome complex; (E) permeabilizing the tissue to allow diffusion of the tagmented fragments and poly-A RNA transcripts to the nanoparticles in the array; (F) capturing the tagmented fragments and poly-A RNA transcripts to the nanoparticles by (i) hybridizing the tagmented fragments to the transposome hybridization region of the first set of oligos, and (ii) hybridizing the poly-A RNA transcripts to the oligo(dT) sequence of the second set of oligos; (G′) processing the first set of oligos comprising captured tagmented fragments to make nanoparticle bound genomic library constructs for sequencing; (G″) reverse transcribing the second set of oligos comprising captured poly-A RNA transcripts in the presence of a single stranded template switch oligo to make nanoparticle bound cDNA library constructs for sequencing; (H) releasing the genomic library constructs and the cDNA library constructs from the nanoparticles by cleaving the selectively cleavable linker (e.g., by using heat and added biotin); (I) amplifying the genomic library constructs and cDNA library constructs using PCR and splitting the amplified products into two portions comprising both amplified constructs; (J) amplifying a first portion of the amplified constructs with a first adapter primer (e.g., a P5 primer, or a P7 primer) and a second adapter primer (e.g., a P5 primer or a P7 primer) to form an ATAC-Seq library, wherein the first adapter primer and the second adapter primer bind to adapter sequences, and wherein the first adapter primer and/or the second adapter primer further comprises an index sequence (e.g., i5 sequence, i7 sequence, etc.) and/or a sequencing primer site sequence (e.g., a R1 sequencing primer site sequence (e.g., SEQ ID NO:5-6), a R2 sequencing primer site sequence (e.g., SEQ ID NO:7-8), etc.); (J′) tagmentating a second portion of the amplified constructs with a primer comprising the sequence primer sequence and TSM and then amplifying the tagmented amplified library constructs using PCR with the first adapter primer comprising a sequencing primer site sequence and the second adapter primer comprising an index sequence and a sequencing primer site sequence to form an RNA-Seq library, wherein the first adapter primer and the second adapter primer have difference sequence primer sequences; and (K) obtaining multi-omics information by sequencing the ATAC-Seq library and RNA-Seq library using a sequencer and mapping the sequencing reads with x,y positions of the nanoparticles. In a further embodiment, the nanoparticles are beads. In another embodiment or a further embodiment, the transposome complex is a TN5 transposome complex. In another embodiment or a further embodiment, the spatial address sequences are decoded by using a decoding-by-hybridization method. In another embodiment or a further embodiment, the cell membranes are lysed by using a mild detergent (e.g., NP40, Tween 20, Bile Salts, Triton X100, and (3-((3-cholamidopropyl)dimethylammonio)-1-propanesulfonate) (CHAPS). In another embodiment or a further embodiment, the tissue is permeabilized by using a detergent (e.g., Tween 20 and/or Triton X100) and Proteinase K. In another embodiment or a further embodiment, the captured tagmented fragments are processed by using top strand ligation, hybridization to Y-shaped adaptors, and gap-filling ligations steps (e.g., a DNA polymerase lacking strand-displacement activity, dNTPs, and a DNA ligase). In another embodiment or a further embodiment, prior to sequencing the library constructs are amplified using PCR. In another embodiment or a further embodiment, the sequencer utilizes sequencing by synthesis technology. In another embodiment or a further embodiment, the hybridization region of the transposome is initially blocked by use of an accessory oligo that is then removed in step (E). In another embodiment or a further embodiment, steps (G) and (G′) are performed sequentially or simultaneously. In another embodiment or a further embodiment, steps (J) and (J′) are performed sequentially or simultaneously. In another embodiment or a further embodiment, the oligo d(T) sequence comprises 5, 6, 7, 8, 9, 10, 11, 12, 13, 14, 15, 16, 17, 18, 19, 20, 21, 22, 23, 24, 25, 26, 27, 28, 29, or 30 nucleotides, or a range of nucleotides that includes or is between any two of the foregoing numbers (e.g., 16 to 20 nucleotides).

In a particular embodiment, the disclosure further provides a spatial transcriptomics method using a beadchip, comprising: (A) loading beads into nanowells of a beadchip, wherein the beads comprise oligos comprising an adapter sequence (e.g., a P5 adapter sequence (SEQ ID NO:1), a P7 adapter sequence (SEQ ID NO:2), etc), a spatial address sequence, an optional sequence primer site (e.g., a R1 sequence primer site (e.g., SEQ ID NO:5-6), a R2 sequence primer site (e.g., SEQ ID NO:7-8), etc.), and an oligo(dT) sequence; (B) decoding the spatial address sequences of the oligos to determine the x,y position of the beads; (C) placing tissue into sectioned areas (e.g., hyb-seal sectioned areas) of the beadchip, and lysing the cell membranes to access poly-A RNA transcripts in the tissue; (D) capturing the poly-A RNA transcripts to the beads; (E) reverse transcribing the oligos comprising captured poly-A RNA transcripts: (i) in the presence of a single stranded template switch oligo to make a bead bound cDNA library constructs for sequencing, or (ii) further performing a single strand ligation reaction with a single stranded oligo to make a bead bound cDNA library constructs for sequencing; (F) amplifying from the sectioned areas (e.g., hyb-seal sectioned areas) the bead bound cDNA library constructs using PCR with an adapter primer (e.g., a P5 adapter primer, a P7 adapter primer, etc.); and (G) obtaining transcriptomics information by sequencing the amplified cDNA library constructs using a sequencer and mapping the sequencing reads with x,y positions of the beads in the beadchip. In a further embodiment, the beadchip has a feature density (feature/mm²) of greater than 300000. In another embodiment or a further embodiment, the beads comprise lanthanide nanophosphor labels.

In a certain embodiment, the disclosure provides a spatial multi-omics method to detect targeted RNA and proteins, comprising: (A) loading multiple sets of lanthanide nanophosphor labeled beads into an array, wherein a first set of beads comprise oligos comprising an adapter sequence (e.g., a P5 adapter sequence (SEQ ID NO:1), a P7 adapter sequence (SEQ ID NO:2), etc), a spatial address sequence, an optional sequence primer site (e.g., a R1 sequence primer site (e.g., SEQ ID NO:5-6), a R2 sequence primer site (e.g., SEQ ID NO:7-8), etc.), and an oligo(dT) sequence, and a second set of beads that comprise oligos comprising a spatial address sequence, a UMI sequence, and a capture sequence; (B) placing tissue on top of the beads, and lysing the cell membranes to access poly-A RNA transcripts and proteins in the tissue; (C) adding antibodies comprising a barcode nucleotide sequence which bind with specificity to protein targets; (D) capturing the poly-A RNA transcripts to the first set of beads; (D′) capturing the antibodies to the second set of beads by hybridizing the barcode nucleotide sequence of the antibodies to the capture sequence; (E) reverse transcribing the oligos comprising captured poly-A RNA transcripts with oNTPS; (F) adding a primer which binds to oligos on the sets of beads and extending the primers with oNTPS; (G) adding a dsDNA dye that binds to the extended primer sequences; and (H) detecting targeted RNA and proteins by imaging and decoding the sets of beads. In a further embodiment, the dsDNA dye is a cyanine-based dye (e.g., PicoGreen). In another embodiment or a further embodiment, the imaging and decoding the sets of beads is performed simultaneously. In another embodiment or a further embodiment, steps (D) and (D′) are performed sequentially or simultaneously.

In a particular embodiment, the disclosure also provides a method for in situ decoding of spatially addressed nano-particles, comprising: (A) infusing oligo-coated nanoparticles into tissue, the oligo-coated nanoparticles comprising attached oligos comprising an adapter sequence (e.g., a P5 adapter sequence (SEQ ID NO:1), a P7 adapter sequence (SEQ ID NO:2), etc), a sequence primer site (e.g., a R1 sequence primer site (e.g., SEQ ID NO:5-6), a R2 sequence primer site (e.g., SEQ ID NO:7-8), etc.), a spatial address sequence, and an oligo d(T) sequence; (B) capturing mRNA in situ by hybridizing mRNA in the tissue with the oligo d(T) sequence of the oligos; (C) reverse transcribing the oligos comprising captured poly-A RNA transcripts in situ to form a nanoparticle bound cDNA construct; (D) mapping the cDNA construct in situ by: (i) direct in situ sequencing of the spatial address sequence; or (ii) using decoding-by-hybridization approach to decode the spatial address sequence in situ; and (E) digesting the tissue with a detergent (e.g., Tween 20 and/or Triton X100) and Proteinase K, and isolating the tagged constructs; (F) separating the cDNA construct from the nanoparticle, library prepping the cDNA construct and sequencing the cDNA construct.

In a certain embodiment, the disclosure further provides a method for in situ decoding of spatially addressed nano-particles, comprising: (A) infusing nanoparticles comprising multiple sets of immobilized oligos into a tissue, the multiple sets of immobilized oligos comprising: a first set of oligos comprising an adapter sequence (e.g., a P5 adapter sequence (SEQ ID NO:1), a P7 adapter sequence (SEQ ID NO:2), etc), a sequence primer site (e.g., a R1 sequence primer site (e.g., SEQ ID NO:5-6), a R2 sequence primer site (e.g., SEQ ID NO:7-8), etc.), a spatial address sequence, and an oligo d(T) sequence, a second set of oligos comprising a spatial address sequence and a Sbs/ME sequence, an optional third set of oligos comprising a an adapter sequence (e.g., a P5 adapter sequence (SEQ ID NO:1), a P7 adapter sequence (SEQ ID NO:2), etc), a spatial address sequence, and a Sbs/ME sequence, wherein the first and third set of oligos are attached to the nanoparticles by a selectively cleavable linker (e.g., biotin-based molecules, PC Linker, and a recognition site for a rare-cutter enzyme), and wherein the second set of oligos are not attached to the nanoparticles by a selectively cleavable linker; (B) capturing mRNA in situ by hybridizing mRNA in the tissue with the oligo d(T) sequence of the first set of oligos; (C) reverse transcribing the oligos comprising captured poly-A RNA transcripts in situ to form a nanoparticle bound cDNA construct; (D) extracting ex situ the cDNA construct from the nanoparticle by cleaving the selectively cleavable linker and separating the cDNA construct from the nanoparticle; (E) mapping the second set of oligos in situ by: (i) direct in situ sequencing of the spatial address sequence; or (ii) using decoding-by-hybridization approach to decode the spatial address sequence in situ; and (F) library prepping the cDNA construct and sequencing the cDNA construct. In a further embodiment, the oligo-coated nanoparticles are functionalized with biological triggers for endocytosis and intracellular transport, or with targeting moieties or ligands for specific subcellular organelles. In another embodiment or a further embodiment, the oligo-coated nanoparticles comprise lanthanide nanophosphor labels or Q-dot particles.

In a particular embodiment, the disclosure provides a spatial genomics method using ex situ spatial capture on a beadchip, comprising: (A) loading beads into nanowells of a beadchip, wherein the beads comprise oligos that attached to the nanoparticles through a selectively cleavable linker (e.g., biotin-based molecules, PC Linker, and a recognition site for a rare-cutter enzyme), the oligos comprising a first adapter sequence (e.g., a P5 adapter sequence (SEQ ID NO:1), a P7 adapter sequence (SEQ ID NO:2), etc), a spatial address sequence, and a first capture sequence; (B) decoding the spatial address sequences of the oligos to determine the x,y position of the beads; (C) placing tissue into sectioned areas of the beadchip, lysing the cell membranes, and adding non-tethered pool of oligos comprising a second adapter sequence (e.g., a P5 adapter sequence (SEQ ID NO:1), a P7 adapter sequence (SEQ ID NO:2), etc), an optional UMI sequence, a sample index sequence, and a second capture sequence, wherein the first and second capture sequence bind to different portions of a biomolecule; (D) capturing the biomolecule to the first and second capture sequence of the oligos; (E) extending and/or ligating the first capture sequence to the second capture sequence to form a construct which comprises the first adapter sequence, the spatial address sequence, the first capture sequence, a gap or bridge molecule of interest, the second capture sequence, the optional UMI sequence, and the second adapter sequence; (F) obtaining ex situ spatial genomics information of the tissue by sequencing the construct using a sequencer and mapping the sequencing reads with x,y positions of the beads. In a further embodiment, the first capture sequence and the second capture sequence having complementary sequences to a targeted gene. In another embodiment or a further embodiment, the gap or bridge molecule of interest comprise nucleotides up to a thousand nucleotides in length

In a certain embodiment, the disclosure provides a spatial genomics Assay for Transposase-Accessible Chromatin with high-throughput sequencing (ATAC-Seq) method, comprising: (A) providing a substrate which comprises oligos attached to the substrate through a selectively cleavable linker, the oligos comprising an adapter sequence, a spatial address sequence, and a transposome hybridization region, wherein if the substrate is an ordered substrate, then the spatial address sequence of the oligos is optional; (B) decoding the spatial address sequences of the oligos to determine the x,y positions of the oligos on the substrate, wherein if the substrate is an ordered substrate, then step (B) is optional; (C) placing tissue on top of the substrate, and lysing the cell membranes to access chromatin regions in the tissue; (D) tagmentating the chromatin regions with a transposome complex to form tagmented fragments, and removing the transposome complex; (E) permeabilizing the tissue to allow diffusion of the tagmented fragments to the substrate; (F) capturing the tagmented fragments to the substrate by hybridizing the tagmented fragments to the transposome hybridization region of the oligos; (G) processing the captured tagmented fragments to make substrate bound genomic library constructs for sequencing; (H) releasing the genomic library constructs from the substrate by selectively cleaving the linker; (I) obtaining spatial genomics information of the tissue by sequencing the genomic library constructs using a sequencer and mapping the sequencing reads with x,y positions of the oligos. In a further embodiment, the substrate is a plate, a multiwell plate, a slide, a flowcell, or nanoparticles. In another embodiment, the substrate is a patterned substrate comprising areas of immobilized oligos separated by interstitial areas lacking immobilized oligos. In yet another embodiment, the substrate is a patterned substrate comprising islands or clusters of immobilized oligos separated by interstitial areas lacking immobilized oligos, and wherein each island or cluster of immobilized oligos has a spatial address sequence that is different from the spatial address sequence of the other islands or clusters of immobilized oligos. In a further embodiment, the substrate is an ordered substrate and the x,y positions of the oligos on the substrate are predetermined or can be readily determined without having to decode the spatial address sequences in step (B). In yet a further embodiment, the transposome complex is a TN5 transposome complex. In another embodiment, the spatial address sequences are decoded by using a decoding-by-hybridization method. In yet another embodiment, the cell membranes are lysed by using a mild detergent. Examples of mild detergent include, but are not limited to, NP40, Tween 20, Bile Salts, Triton X100, and (3-((3-cholamidopropyl) dimethylammonio)-1-propanesulfonate) (CHAPS). In a further embodiment, the tissue is permeabilized by using a detergent (e.g., Tween 20 and/or Triton X100) and Proteinase K. In yet a further embodiment, the captured tagmented fragments are processed by using top strand ligation, hybridization to Y-shaped adaptors, and gap-filling ligations steps. In a certain embodiment, prior to sequencing the library constructs are amplified using PCR. In a further embodiment, the sequencer utilizes sequencing-by-synthesis technology. In yet a further embodiment, the hybridization region of the transposome complex is initially blocked by use of an accessory oligo that is then removed in step (E).

In a particular embodiment, the disclosure also provides a spatial multi-omics Assay for Transposase-Accessible Chromatin with high-throughput sequencing (ATAC-Seq) and RNA-Seq method, comprising: (A) providing a substrate which comprises multiple sets of immobilized oligos attached to the substrate through selectively cleavable linker(s), wherein the substrate comprises a first set of oligos which comprises a first adapter sequence, a spatial address sequence, and a transposome complex hybridization region, and a second set of oligos that comprises a second adapter sequence, a sequencing primer site sequence, a spatial address sequence, a unique molecular identifier (UMI) sequence, and an oligo(dT) sequence, wherein if the substrate is an ordered substrate, then the spatial address sequence of the first set of oligos, and/or second set of oligos are optional; (B) decoding the spatial address sequences of the oligos to determine the x,y position of the oligos on the substrate, wherein if the substrate is an ordered substrate, then step (B) is optional; (C) placing tissue on top of the substrate, and lysing the cell membranes to access poly-A RNA transcripts and chromatin regions in the tissue; (D) tagmentating the chromatin regions with a first transposome complex to form tagmented fragments, and removing the first transposome complex; (E) permeabilizing the tissue to allow diffusion of the tagmented fragments and poly-A RNA transcripts to the substrate; (F) capturing the tagmented fragments and poly-A RNA transcripts to the substrate by (i) hybridizing the tagmented fragments to the transposome hybridization region of the first set of oligos, and (ii) hybridizing the poly-A RNA transcripts to the oligo(dT) sequence of the second set of oligos; (G′) processing the first set of oligos comprising captured tagmented fragments to make substrate bound genomic library constructs for sequencing; (G″) reverse transcribing the second set of oligos comprising captured poly-A RNA transcripts in the presence of a single stranded template switch oligo to make substrate bound cDNA library constructs for sequencing; (H) releasing the genomic library constructs and the cDNA library constructs from the substrate by selectively cleaving the linker(s); (I) amplifying the genomic library constructs and cDNA library constructs using PCR and splitting the amplified products into two portions comprising both amplified constructs; (J) amplifying a first portion of the amplified constructs with a first adapter primer and a second adapter primer to form an ATAC-Seq library, wherein the first adapter primer and the second adapter primer bind to first adapter sequence and second adapter sequence, respectively, and wherein the first adapter primer and/or the second adapter primer further comprises an index sequence and/or a sequencing primer site sequence; (J′) tagmentating a second portion of the amplified constructs with a primer comprising the sequence primer sequence and a transposome complex (TSM) and then amplifying the tagmented amplified library constructs using PCR with the first adapter primer comprising a sequencing primer site sequence and the second adapter primer comprising an index sequence and a sequencing primer site sequence to form an RNA-Seq library, wherein the first adapter primer and the second adapter primer have difference sequence primer sequences; and (K) obtaining multi-omics information by sequencing the ATAC-Seq library and RNA-Seq library using a sequencer and mapping the sequencing reads with x,y positions of the oligos. In another embodiment, the substrate is a plate, a multiwell plate, a slide, a flowcell, or nanoparticles. In yet another embodiment, the substrate is a patterned substrate comprising areas of immobilized oligos separated by interstitial areas lacking immobilized oligos. In a further embodiment, the substrate is a patterned substrate comprising islands or clusters of the two sets of immobilized oligos separated by interstitial areas lacking immobilized oligos, and wherein each island or cluster of the immobilized oligos has a spatial address sequence that is different from the spatial address sequence of the other islands or clusters of immobilized oligos. In yet a further embodiment, the substrate is an ordered substrate and the x,y positions of the oligos on the substrate are predetermined or can be readily determined without having to decode the spatial address sequences in step (B). In another embodiment, the transposome complex is a TN5 transposome complex. In yet another embodiment, the spatial address sequences are decoded by using a decoding-by-hybridization method. In a further embodiment, the cell membranes are lysed by using a mild detergent. In yet a further embodiment, the tissue is permeabilized by using a detergent and Proteinase K. In another embodiment, the captured tagmented fragments are processed by using top strand ligation, hybridization to Y-shaped adaptors, and gap-filling ligations steps. In yet another embodiment, prior to sequencing the library constructs are amplified using PCR. In a certain embodiment, the sequencer utilizes sequencing-by-synthesis technology. In another embodiment, the hybridization region of the transposome complex is initially blocked by use of an accessory oligo that is then removed in step (E). In yet another embodiment, steps (G) and (G′) are performed sequentially or concurrently. In a further embodiment, steps (J) and (J′) are performed sequentially or concurrently. In yet a further embodiment, the oligo d(T) sequence comprises 16 to 20 nucleotides.

In a particular embodiment, the disclosure also provides a spatial transcriptomics method, comprising: (A) providing a substrate comprising features, wherein oligos are immobilized on the features of the substrate, wherein the oligos comprise an adapter sequence, a spatial address sequence, an optional sequence primer site, and an oligo(dT) sequence, wherein if the substrate is an ordered substrate, then the spatial address sequence of the oligos is optional; (B) decoding the spatial address sequences of the oligos to determine their x,y position in the substrate, wherein if the substrate is an ordered substrate, then step (B) is optional; (C) placing tissue onto the substrate, and lysing the cell membranes to access poly-A RNA transcripts in the tissue; (D) capturing the poly-A RNA transcripts to the oligos; (E) reverse transcribing the oligos comprising captured poly-A RNA transcripts: (i) in the presence of a single stranded template switch oligo to make substrate bound cDNA library constructs for sequencing, or (ii) further performing a single strand ligation reaction with a single stranded oligo to make substrate bound cDNA library constructs for sequencing; (F) amplifying the oligo bound cDNA library constructs using PCR with an adapter primer; (G) obtaining transcriptomics information by sequencing the amplified cDNA library constructs using a sequencer and mapping the sequencing reads with x,y positions of the oligos on the features of the substrate. In another embodiment, the substrate is a microarray, a plate, a multiwell plate, or a flowcell. In yet another embodiment, the substrate is an ordered substrate and the x,y positions of the oligos on the features of the substrate are predetermined or can be readily determined without having to decode the spatial address sequences in step (B). In a further embodiment, the substrate has a feature density (feature/mm²) of greater than 300000. In yet a further embodiment, the features of the substrate and/or oligos further comprise lanthanide nanophosphor labels.

In a particular embodiment, the disclosure provides a spatial multi-omics method to detect targeted RNA and proteins, comprising: (A) providing a substrate comprising features having lanthanide nanophosphor labels, wherein the features comprise multiple sets of immobilized oligos, wherein a first set of oligos comprise an adapter sequence, a spatial address sequence, an optional sequence primer site, and an oligo(dT) sequence, and a second set oligos that comprise a spatial address sequence, a UMI sequence, and a capture sequence, and wherein if the substrate is an ordered substrate, then the spatial address sequence of the first set of oligos and/or the second set of oligos are optional; (B) decoding the spatial address sequences of the oligos to determine their x,y position in the substrate, wherein if the substrate is an ordered substrate, then step (B) is optional; (C) placing tissue on top of the substrate, and lysing the cell membranes to access poly-A RNA transcripts and proteins in the tissue; (D) adding antibodies comprising a barcode nucleotide sequence which bind with specificity to a targeted protein(s); (E) capturing the poly-A RNA transcripts to the first set of oligos; (E′) capturing the antibodies to the second set of oligos by hybridizing the barcode nucleotide sequence of the antibodies to the capture sequence; (F) reverse transcribing the oligos comprising captured poly-A RNA transcripts with oNTPS; (G) adding a primer which binds to oligos on the features of the substrate and extending the primers with oNTPS; (H) adding a dsDNA dye that binds to the extended primer sequences; and (I) detecting targeted RNA and proteins by imaging, and determining the x,y positions of the first and second sets of oligos on the features of the substrate. In another embodiment, the substrate is a microarray, a plate, a multiwell plate, or a flowcell. In yet another embodiment, the substrate is an ordered substrate and the x,y positions of the oligos on the features of the substrate are predetermined or can be readily determined without having to decode the spatial address sequences in step (B). In a further embodiment, the substrate has a feature density (feature/mm²) of greater than 300000. In yet a further embodiment, the dsDNA dye is a cyanine-based dye. In a certain embodiment, the cyanine-based dye is selected from SYBR Green I, PicoGreen, SYBR Safe, SYBR Gold, thiazole orange, oxazole yellow, Safe-Green and Chai Green. In a further embodiment, steps (D) and (D′) are performed sequentially or concurrently.

In a particular embodiment, the disclosure also provides a method for in situ decoding of spatially addressed oligos, comprising: (A) infusing an oligo-coated substrate into tissue, the oligo-coated substrate comprising immobilized oligos comprising an adapter sequence, a sequence primer site, a spatial address sequence, and an oligo d(T) sequence; (B) capturing mRNA in situ by hybridizing mRNA in the tissue with the oligo d(T) sequence of the oligos; (C) reverse transcribing the oligos that comprise captured poly-A RNA transcripts in situ to form a substrate bound cDNA construct; (D) mapping the cDNA construct in situ by: (i) direct in situ sequencing of the spatial address sequence; or (ii) using decoding-by-hybridization approach to decode the spatial address sequence in situ; and (E) digesting the tissue with a detergent and Proteinase K, and isolating the substrate bound cDNA construct; (F) separating the cDNA construct from the substrate, library prepping the cDNA construct and sequencing the cDNA construct. In a further embodiment, the substrate is from 10 nm to 10 μm in size. In yet a further embodiment, the substrate is functionalized with biological triggers for endocytosis and intracellular transport, or with targeting moieties or ligands for specific subcellular organelles. In another embodiment, the substrate or oligos comprise lanthanide nanophosphor labels or Q-dot particles.

In a particular embodiment, the disclosure further provides, a method for in situ decoding of spatially addressed oligos, comprising: (A) infusing an oligo-coated substrate into tissue, the oligo-coated substrate comprising multiple sets of immobilized oligos sets, the multiple sets of immobilized oligos comprising: a first set of oligos comprising a first adapter sequence, a sequence primer site, a spatial address sequence, and an oligo d(T) sequence, a second set of oligos comprising a spatial address sequence and a Sbs/ME sequence, an optional third set of oligos comprising the first adapter sequence, a spatial address sequence, and a Sbs/ME sequence, wherein the first and third set of oligos are attached to the substrate by a selectively cleavable linker, and wherein the second set of oligos are not attached to the substrate by a selectively cleavable linker; (B) capturing mRNA in situ by hybridizing mRNA in the tissue with the oligo d(T) sequence of the first set of oligos; (C) reverse transcribing the oligos comprising captured poly-A RNA transcripts in situ to form a substrate bound cDNA construct; (D) extracting ex situ the cDNA construct from the substrate by selectively cleaving the cleavable linker and separating the cDNA construct from the substrate; (E) mapping the second set of oligos in situ by: (i) direct in situ sequencing of the spatial address sequence; or (ii) using decoding-by-hybridization approach to decode the spatial address sequence in situ; and (F) library prepping the cDNA construct and sequencing the cDNA construct. In another embodiment, the substrate is from 10 nm to 10 μm in size. In yet another embodiment, the substrate is functionalized with biological triggers for endocytosis and intracellular transport, or with targeting moieties or ligands for specific subcellular organelles. In yet another embodiment, the substrate or multiple sets of oligos comprise lanthanide nanophosphor labels or Q-dot particles.

In a particular embodiment, the disclosure provides a spatial genomics method using ex situ spatial capture on a substrate, comprising: (A) providing a substrate comprising features, wherein the features comprise immobilized oligos that attached to the features of the substrate through a selectively cleavable linker, the oligos comprising a first adapter sequence, a spatial address sequence, and a first capture sequence, wherein if the substrate is an ordered substrate, then the spatial address sequence of the oligos is optional; (B) decoding the spatial address sequences of the oligos to determine the x,y position of the beads, wherein if the substrate is an ordered substrate, then step (B) is optional; (C) placing tissue on the substrate, lysing the cell membranes, and adding non-tethered pool of oligos comprising a second adapter sequence, an optional UMI sequence, a sample index sequence, and a second capture sequence, wherein the first and second capture sequence bind to different portions of a biomolecule; (D) capturing the biomolecule to the first and second capture sequence of the oligos; (E) extending and/or ligating the first capture sequence to the second capture sequence to form a construct which comprises the first adapter sequence, the spatial address sequence, the first capture sequence, a gap or bridge molecule of interest, the second capture sequence, the optional UMI sequence, and the second adapter sequence; and (F) obtaining ex situ spatial genomics information of the tissue by sequencing the construct using a sequencer and mapping the sequencing reads with the determined x,y positions of the oligos. In a further embodiment, the substrate is a microarray, a plate, a multiwell plate, or a flowcell. In another embodiment, the substrate is an ordered substrate and the x,y positions of the oligos on the features of the substrate are predetermined, or can be readily determined without having to decode the spatial address sequences in step (B). In yet a further embodiment, the substrate has a feature density (feature/mm²) of greater than 300000. In another embodiment, the first capture sequence and the second capture sequence having complementary sequences to a targeted gene. In yet another embodiment, the gap or bridge molecule of interest comprise nucleotides up to a thousand nucleotides in length.

### BRIEF DESCRIPTION OF THE DRAWINGS

FIG. 1 presents a diagram of a nanoparticle-based array of the disclosure that can be used in various spatial omic systems and platforms, including, but not limited to, spatial proteomic systems and platforms; spatial transcriptomic systems and platforms; spatial agrigenomic systems and platforms; spatial epigenomics systems and platforms; spatial phenomic systems and platforms; spatial ligandomic systems and platforms; and spatial multiomic systems and platforms. As diagramed, each nanoparticle comprises a unique barcode that identifies the nanoparticle by location and by sample type.

FIG. 2 presents an embodiment of an array-based design for carrying out various spatial omic systems and platforms disclosed herein. As diagrammed, a bead or nanowell comprises address sequence(s) (e.g., target, sample ID, and linker sequences); and capture sequences that are complementary to: specific protein bound oligonucleotides; RNA/DNA; or capture sequences. For example, the capture sequence may hybridize to an oligonucleotide which has a linked antibody or a linked scFC domain that allows for analyte capture with downstream signal generation (e.g., protein capture). Alternatively, the capture sequence can hybridize to an oligonucleotide for tag capture and secondary readouts (e.g., antibody-linked transposome, other DNA signal generation method). In anther example, the capture sequence can bind to genomic sequences for direct capture applications (e.g., WGA/similar).

FIG. 3 diagrams a sample workflow for spatial omic systems and platforms utilizing nanoparticles coated with barcoded oligonucleotides that can bind mRNA (and/or protein or DNA). 3D in situ imaging of the beads can be used to decode the barcode. mRNA can be copied onto the barcoded oligonucleotides through reverse transcription; the cDNA molecules are then extracted from the tissue, followed by library preparation and sequencing to generated spatially localized omic information (e.g., spatially localized transcriptomic information).

FIG. 4 shows a scheme using tissue expansion and infusion of barcoded circular oligonucleotides followed by in situ rolling circle amplification to generate barcoded nanoballs. This embodiment of a method of the disclosure is advantageous as it may lead to improved tissue penetration by the barcoded oligonucleotides, as single barcoded oligonucleotides are smaller than an oligo-coated nanoparticle.

FIG. 5 illustrates that having two oligonucleotides on the nanoparticle (a decode oligo and a capture oligo) could be advantageous for the following reasons: a reverse transcription reaction can be performed and the cDNA extracted prior to decoding the barcode, so that the cDNA molecules would not have to be subjected to decode reaction (the decode oligo stays with the nanoparticle). The decode oligo/capture oligo stoichiometry could be different and the oligos themselves could be changed to fit the need. To overcome density issues (i.e., if the nanoparticles are too close, it may be difficult to image individual beads), the decode primer site could be made into several versions (e.g., sbs3.1, sbs3.2, sbs3.9); then multiple rounds of hybridization and decode by sequencing. The average density in any round would be 1/10 of the actual bead density. The process can handle multi-modal or co-assay nanoparticles with different oligonucleotide types (the illustration showing an mRNA capture oligo and a transposon for Tn5-based tagmentation of DNA).

FIG. 6A-C presents an embodiment of a process for decoding different sequences on a randomly ordered bead array. (A-B) The process starts by hybridizing labeled decoding nucleotides with the address segments on the beads at high concentrations, which allows for rapid hybridizations, followed by washing to remove non-specific signals and the background. (C) After the fluorescence readout, several rehybridization steps with other decoding nucleotide sets are performed, until there is sufficient data to unambiguously determine the identity of each bead.

FIG. 7 provides an exemplary workflow for carrying out spatial transcriptomics using tissue in situ. As shown, beads comprising an address and capture sequence are placed into an array, and their position are decoded using sequential hybridization and error-correcting strategy. After which, a frozen tissue section is placed on the decoded slide, stained and imaged, RNA is captured and then profiled by next generation sequencing.

FIG. 8 provides a workflow diagram showing how methods for spatial omics disclosed herein can be extended to Assay for Transposase-Accessible Chromatin with high-throughput sequencing (ATAC-Seq) using a bead-based array.

FIG. 9 provides a workflow diagram showing how methods for spatial omics disclosed herein can be modified to perform a co-assay of ATAC-Seq and RNA-Seq using a bead-based array.

FIG. 10 provides embodiments directed to how beads may be modified to carry out the workflow diagram of FIG. 9. In particular, the beads are modified so that 2 types of oligos are attached to each bead, both with the same spatial barcode. One oligo captures gDNA while the other oligo captures mRNA. Assembly of this type of bead is possible by modification of the split-pool technique. By using different linker regions for the two oligos types confers specificity of assembly.

FIG. 11 provides additional embodiments directed to how beads may be modified to carry out the workflow diagram of FIG. 9. In particular, the beads are modified so that 2 types of oligos are attached to each bead, both with the same spatial barcode. One oligo captures gDNA while the other oligo captures mRNA. Assembly of this type of bead is possible by modification of the split-pool technique. By incorporating blocking/deblocking steps allows the use of the same linker sequences, reducing the number of unique oligos that must be synthesized.

FIG. 12A-C provides an alternative method for spatial omics disclosed herein that utilizes oligonucleotides released from a substrate to capture RNA molecules in situ. In the original implementation, RNA molecules must diffuse from the tissue onto the bead array in order to hybridize with the capture oligos. (A) Reversing this process (i.e., allowing the oligos to diffuse from the bead array into the tissue) may improve capture efficiency as the barcoded oligos are much shorter than the average mRNA transcript and therefore may diffuse more efficiently into the tissue (150 bp vs 3 kb). (B) Applying an electric field with the positive electrode above the tissue may also help drive the oligos deep into the tissue. However, the electric field will also cause the mRNAs to migrate; in theory, the shorter oligos should be able to catch up to the longer mRNA molecules, but shorter mRNA fragments may not be captured. (C) An alternative approach would be to confer a positive charge to the oligos so that application of an electric field below the beads would result in the mRNA migrating towards the beads whereas the positively-charged spatial oligos would migrate towards the tissue. Conferring a positive charge to the oligos could be accomplished by using a positively-charged peptide nucleic acid (PNA) oligo or an oligo attached to a positively charged gold nanoparticle.

FIG. 13 provides a spatial omics assay that utilizes a bead based genotyping strategy for ex situ spatial capture. As shown, a bead is generated which comprises an identifying code of the x,y space (i.e., address sequence) and a first capture sequence (e.g., LSP1) that are tethered to the bead with biotin containing residue(s). The beads are placed in an array and the x,y positions are decoded. Another pool of oligonucleotides is created that are not tethered to a substrate, and which comprise a second capture sequence (LSP2), an index sequence, and optionally an UMI tag. A tissue sample is sectioned and placed on top of the beads and treated to release target molecules (e.g., DNA, RNA, polypeptides) which are then tethered to the beads by hybridizing to the capture sequence. The nontethered pool of the oligonucleotides are then added and hybridize to complementary sequences in the target molecules. After washing to remove unbound oligonucleotides and non-targeted molecules, an extension and ligation step is performed to form a construct of FIG. 14.

FIG. 14 provides the diagram of the construct made in FIG. 13, showing the arrangement of the various sequences making up the construct. The construct is depicted as having an UMI sequence, but the UMI is optional and may not be present.

FIG. 15 diagrams how the x,y positions of the beads of FIG. 13 can be visualized by using decoding methods described herein.

FIG. 16A-B provides embodiments on how a microarray system can be used in the methods disclosed herein for spatial omics. (A) The microarray system can be used for spatial transcriptomics, by comprising beads which contain a tethered oligonucleotide, the oligonucleotide comprising a P7 sequence, an address sequence, an optional sequence primer site sequence, and an oligo dT (OdT) sequence. RNA from a sample is then in situ hybridized to the OdT sequence. The bound RNA is reverse transcribed and a code is then ligated to the end of the transcript with an RNA ligase. Using P5 and P7 primers the transcript is amplified and cluster/sequences. The x,y position of the beads can be determined using the address sequence with a decoding protocol. (B) The microarray system can be used for spatial proteomics, by replacing the OdT sequences with a library of sequences that can capture tagged aptamers or mAb.

FIG. 17 provides a diagram how the systems disclosed herein can be improved to capture antibodies that comprise short barcode sequences.

FIG. 18 provides a diagram of a lanthanide nanophosphor bead-based microarray that can be used for spatial multi-omic (RNA and protein) detection. As shown, the microarray comprises multiple sets of oligonucleotides that can bind different target molecules, e.g., RNA and proteins.

FIG. 19 provides additional embodiments and specifics of the lanthanide nanophosphor bead-based microarray of FIG. 18 for spatial multi-omic applications.

### DETAILED DESCRIPTION

As used herein, the singular forms “a,” “an” and “the” include plural referents unless the content clearly dictates otherwise. Thus, for example, reference to “a protein” includes a mixture of two or more proteins, and the like.

It is to be further understood that where descriptions of various embodiments use the term “comprising,” those skilled in the art would understand that in some specific instances, an embodiment can be alternatively described using language “consisting essentially of” or “consisting of.”

As used herein, the terms “includes,” “including,” “includes,” “including,” “contains,” “containing,” “have,” “having,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter. Similarly, “comprise,” “comprises,” “comprising” “include,” “includes,” and “including” are interchangeable and not intended to be limiting.

Other than in the operating examples, or where otherwise indicated, all numbers expressing quantities of ingredients or reaction conditions used herein should be understood as modified in all instances by the term “about.” The term “about” when used to described embodiments of the disclosure, in connection with percentages means ±1%, ±2%, ±3%, 4%, ±5%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, or 6 to 9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

As used herein, the term “complementary” when used in reference to a polynucleotide is intended to mean a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application, and can be routinely determined by persons skilled in the art, without undue experimentation.

As used herein, the term “dNTP” refers to deoxynucleoside triphosphates. NTP refers to ribonucleotide triphosphates. The purine bases (Pu) include adenine (A), guanine (G) and derivatives and analogs thereof. The pyrimidine bases (Py) include cytosine (C), thymine (T), uracil (U) and derivatives and analogs thereof. Examples of such derivatives or analogs, by way of illustration and not limitation, are those which are modified with a reporter group, biotinylated, amine modified, radiolabeled, alkylated, and the like and also include phosphorothioate, phosphite, ring atom modified derivatives, and the like. The reporter group can be a fluorescent group such as fluorescein, a chemiluminescent group such as luminol, a terbium chelator such as N-(hydroxyethyl)ethylenediaminetriacetic acid that is capable of detection by delayed fluorescence, and the like.

As used herein, the term “hybridization” refers to the process in which two single-stranded polynucleotides bind non-covalently to form a stable double-stranded polynucleotide. A resulting double-stranded polynucleotide is a “hybrid” or “duplex.” Hybridization conditions will typically include salt concentrations of less than about 1 M, more usually less than about 500 mM and may be less than about 200 mM. A hybridization buffer can include a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are usually performed under stringent conditions, i.e., conditions under which a probe will hybridize to its target subsequence but will not hybridize to the other, uncomplimentary sequences. Stringent conditions are sequence-dependent and are different in different circumstances, and may be determined routinely by those skilled in the art.

As used herein, the terms “ligation,” “ligating,” and grammatical equivalents thereof are intended to mean to form a covalent bond or linkage between the termini of two or more nucleic acids, e.g., oligonucleotides and/or polynucleotides, typically in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide. Template driven ligation reactions are described in the following references: U.S. Pat. Nos. 4,883,750; 5,476,930; 5,593,826; and 5,871,921, incorporated herein by reference in their entireties. The term “ligation” also encompasses non-enzymatic formation of phosphodiester bonds, as well as the formation of non-phosphodiester covalent bonds between the ends of oligonucleotides, such as phosphorothioate bonds, disulfide bonds, and the like.

As used herein, the term “nucleic acid” means single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH⁴⁺, trialkylammonium, tetraalkylammonium, Mg²⁺, Na⁺ and the like. A nucleic acid can be a polynucleotide or an oligonucleotide. A nucleic acid may be composed entirely of deoxyribonucleotides, entirely of ribonucleotides, or mixtures thereof. The nucleotide monomer units may comprise any of the nucleotides described herein, including, but not limited to, naturally occurring nucleotides and nucleotides analogs. Nucleic acid typically ranges in size from a few monomeric units, e.g., 5-40, to several thousands of monomeric nucleotide units. Nucleic acids include, but are not limited to, genomic DNA, eDNA, hnRNA, mRNA, rRNA, tRNA, fragmented nucleic acid, nucleic acid obtained from sub-cellular organelles such as mitochondria or chloroplasts, and nucleic acid obtained from microorganisms or DNA or RNA viruses that may be present on or in a biological sample.

As used herein, the term “nucleotide analogs” refers to synthetic analogs having modified nucleotide base portions, modified pentose portions, and/or modified phosphate portions, and, in the case of polynucleotides, modified internucleotide linkages, as generally described elsewhere (e.g., Scheit, Nucleotide Analogs, John Wiley, New York, 1980; Englisch, Angew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1998). Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H⁺, NH⁴⁺, Na⁺, if such counterions are present. Exemplary modified nucleotide base portions include but are not limited to 5-methylcytosine (5mC); C-5-propynyl analogs, including but not limited to, C-5 propynyl-C and C-5 propynyl-U; 2,6-diaminopurine, also known as 2-amino adenine or 2-amino-dA); hypoxanthine, pseudouridine, 2-thiopyrimidine, isocytosine (isoC), 5-methyl isoC, and isoguanine (isoG; see, e.g., U.S. Pat. No. 5,432,272). Exemplary modified pentose portions include, but are not limited to, locked nucleic acid (LNA) analogs including without limitation Bz-A-LNA, 5-Me-Bz-C-LNA, dmf-G-LNA, and T-LNA (see, e.g., The Glen Report, 16(2):5, 2003; Koshkin et al., Tetrahedron 54:3607-30, 1998), and 2′- or 3′-modifications where the 2′- or 3′-position is hydrogen, hydroxy, alkoxy (e.g., methoxy, ethoxy, allyloxy, isopropoxy, butoxy, isobutoxy and phenoxy), azido, amino, alkylamino, fluoro, chloro, or bromo. Modified internucleotide linkages include phosphate analogs, analogs having achiral and uncharged intersubunit linkages (e.g., Sterchak, E. P. et al., Organic Chern., 52:4202, 1987), and uncharged morpholino-based polymers having achiral intersubunit linkages (see, e.g., U.S. Pat. No. 5,034,506). Some internucleotide linkage analogs include morpholidate, acetal, and polyamide-linked heterocycles.

In the context of “polynucleotides,” the terms “variant” and “derivative” as used herein refer to a polynucleotide that comprises a nucleotide sequence of a polynucleotide or a fragment of a polynucleotide, which has been altered by the introduction of nucleotide substitutions, deletions or additions. A variant or a derivative of a polynucleotide can be a fusion polynucleotide which contains part of the nucleotide sequence of a polynucleotide. The term “variant” or “derivative” as used herein also refers to a polynucleotide or a fragment thereof, which has been chemically modified, e.g., by the covalent attachment of any type of molecule to the polynucleotide. For example, but not by way of limitation, a polynucleotide or a fragment thereof can be chemically modified, e.g., by acetylation, phosphorylation, methylation, etc. The variants or derivatives are modified in a manner that is different from naturally occurring or starting nucleotide or polynucleotide, either in the type or location of the molecules attached. Variants or derivatives further include deletion of one or more chemical groups which are naturally present on the nucleotide or polynucleotide. A variant or a derivative of a polynucleotide or a fragment of a polynucleotide can be chemically modified by chemical modifications using techniques known to those of skill in the art, including, but not limited to specific chemical cleavage, acetylation, formulation, etc. Further, a variant or a derivative of a polynucleotide or a fragment of a polynucleotide can contain one or more dNTPs or nucleotide analogs. A polynucleotide variant or derivative may possess a similar or identical function as a polynucleotide or a fragment of a polynucleotide described herein. A polynucleotide variant or derivative may possess an additional or different function compared with a polynucleotide or a fragment of a polynucleotide described herein.

As used herein, the term “double-stranded,” when used in reference to a nucleic acid molecule, means that substantially all of the nucleotides in the nucleic acid molecule are hydrogen bonded to a complementary nucleotide. A partially double stranded nucleic acid can have at least 10%, 25%, 50%, 60%, 70%, 80%, 90% or 95% of its nucleotides hydrogen bonded to a complementary nucleotide.

As used herein, the term “single-stranded,” when used in reference to a nucleic acid molecule, means that essentially none of the nucleotides in the nucleic acid molecule are hydrogen bonded to a complementary nucleotide.

As used herein, the term “gene-specific” or “target specific” when used in reference to a capture probe or other nucleic acid is intended to mean a capture probe or other nucleic acid that includes a nucleotide sequence specific to a targeted nucleic acid, e.g., a nucleic acid from a tissue sample, namely a sequence of nucleotides capable of selectively annealing to an identifying region of a targeted nucleic acid. Gene-specific capture probes can have a single species of oligonucleotide, or can include two or more species with different sequences. Thus, the gene-specific capture probes can be two or more sequences, including 3, 4, 5, 6, 7, 8, 9 or 10 or more different sequences. The gene-specific capture probes can comprise a gene-specific capture primer sequence and a universal capture probe sequence. Other sequences such as sequencing primer site sequences, barcode, unique molecular identifiers, or the like also can be included in a gene-specific capture primer.

In comparison, the term “universal” when used in reference to a capture probe or other nucleic acid is intended to mean a capture probe or nucleic acid having a common nucleotide sequence among a plurality of capture probes. A common sequence can be, for example, a sequence complementary to the same adapter sequence. Universal capture probes are applicable for interrogating a plurality of different polynucleotides without necessarily distinguishing the different species whereas gene-specific capture primers are applicable for distinguishing the different species.

In various embodiments, the capture elements (e.g., capture primers or capture probes or other nucleic acid sequences) can be spaced to (A) spatially resolve nucleic acids within the geometry of a single cell, i.e., multiple capture sites per cell; (B) spatially resolve nucleic acids at about the single cell level, i.e., about 1 capture site per cell. Additionally, capture elements may be spaced as in A or B above, and be: (I) spaced to sample nucleic acids from a sample at regular intervals, e.g., spaced in a grid or pattern such that about every other or every 5th or every 10th cell is sampled, or about every other or every 5th or every 10 group of 2, 3, 4, 5, 6, 7, 8, 9, 10 or more cells is sampled; (II) spaced to capture samples from substantially all available cells in one or more regions of a sample; or (III) spaced to capture samples from substantially all available cells in the sample.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, or ligation chain reaction. An amplicon can be a nucleic acid molecule having a single copy of a particular nucleotide sequence (e.g., a PCR product) or multiple copies of the nucleotide sequence (e.g., a concatemeric product of RCA). A first amplicon of a target nucleic acid can be a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

The number of template copies or amplicons that can be produced can be modulated by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. The number of copies of a nucleic acid template can be at least 1, 10, 100, 200, 500, 1000, 2000, 3000, 4000, 5000, 6000, 7000, 8000, 9000 and 10,000 copies, or a range that includes or is between any two of the foregoing numbers, and can be varied depending on the particular application.

As used herein, the term “tagmentation,” “tagment,” or “tagmenting” refers to transforming a nucleic acid, e.g., a DNA, into adaptor-modified templates in solution ready for cluster formation and sequencing by the use of transposase mediated fragmentation and tagging. This process often involves the modification of the nucleic acid by a transposome complex comprising transposase enzyme complexed with adaptors comprising transposon end sequence. Tagmentation results in the simultaneous fragmentation of the nucleic acid and ligation of the adaptors to the 5′ ends of both strands of duplex fragments. Following a purification step to remove the transposase enzyme, additional sequences are added to the ends of the adapted fragments by PCR.

A “transposase” means an enzyme that is capable of forming a functional complex with a transposon end-containing composition (e.g., transposons, transposon ends, transposon end compositions) and catalyzing insertion or transposition of the transposon end-containing composition into the double-stranded target nucleic acid with which it is incubated, for example, in an in vitro transposition reaction. A transposase as presented herein can also include integrases from retrotransposons and retroviruses. Transposases, transposomes and transposome complexes are generally known to those of skill in the art, as exemplified by the disclosure of US Pat. Publ. No. 2010/0120098, the content of which is incorporated herein by reference in its entirety. Although many embodiments described herein refer to Tn5 transposase and/or hyperactive Tn5 transposase, it will be appreciated that any transposition system that is capable of inserting a transposon end with sufficient efficiency to 5′-tag and fragment a target nucleic acid for its intended purpose can be used in the present invention. In particular embodiments, a preferred transposition system is capable of inserting the transposon end in a random or in an almost random manner to 5′-tag and fragment the target nucleic acid.

As used herein, the term “transposition reaction” refers to a reaction wherein one or more transposons are inserted into target nucleic acids, e.g., at random sites or almost random sites. Essential components in a transposition reaction are a transposase and DNA oligonucleotides that exhibit the nucleotide sequences of a transposon, including the transferred transposon sequence and its complement (the non-transferred transposon end sequence) as well as other components needed to form a functional transposition or transposome complex. The DNA oligonucleotides can further comprise additional sequences (e.g., adaptor or primer sequences) as needed or desired. In some embodiments, the method provided herein is exemplified by employing a transposition complex formed by a hyperactive Tn5 transposase and a Tn5-type transposon end (Goryshin and Reznikoff, 1998, J. Biol. Chem., 273: 7367) or by a MuA transposase and a Mu transposon end comprising R1 and R2 end sequences (Mizuuchi, 1983, Cell, 35: 785; Savilahti et al., 1995, EMBO J., 14: 4893). However, any transposition system that is capable of inserting a transposon end in a random or in an almost random manner with sufficient efficiency to 5′-tag and fragment a target DNA for its intended purpose can be used in the present invention. Examples of transposition systems known in the art which can be used for the present methods include but are not limited to Staphylococcus aureus Tn552 (Colegio et al., 2001, J Bacterid., 183: 2384-8; Kirby et al., 2002, Mol Microbiol, 43: 173-86), TyI (Devine and Boeke, 1994, Nucleic Acids Res., 22: 3765-72 and International Patent Application No. WO 95/23875), Transposon Tn7 (Craig, 1996, Science. 271: 1512; Craig, 1996, Review in: Curr Top Microbiol Immunol, 204: 27-48), TnIO and ISIO (Kleckner et al., 1996, Curr Top Microbiol Immunol, 204: 49-82), Mariner transposase (Lampe et al., 1996, EMBO J., 15: 5470-9), Tci (Plasterk, 1996, Curr Top Microbiol Immunol, 204: 125-43), P Element (Gloor, 2004, Methods Mol Biol, 260: 97-114), TnJ (Ichikawa and Ohtsubo, 1990, J Biol Chem. 265: 18829-32), bacterial insertion sequences (Ohtsubo and Sekine, 1996, Curr. Top. Microbiol. Immunol. 204:1-26), retroviruses (Brown et al., 1989, Proc Natl Acad Sci USA, 86: 2525-9), and retrotransposon of yeast (Boeke and Corces, 1989, Annu Rev Microbiol. 43: 403-34). The method for inserting a transposon end into a target sequence can be carried out in vitro using any suitable transposon system for which a suitable in vitro transposition system is available or that can be developed based on knowledge in the art. In general, a suitable in vitro transposition system for use in the methods provided herein requires, at a minimum, a transposase enzyme of sufficient purity, sufficient concentration, and sufficient in vitro transposition activity and a transposon end with which the transposase forms a functional complex with the respective transposase that is capable of catalyzing the transposition reaction. Suitable transposase transposon end sequences that can be used in the invention include but are not limited to wild-type, derivative or mutant transposon end sequences that form a complex with a transposase chosen from among a wild-type, derivative or mutant form of the transposase.

As used herein, the term “transposome complex” refers to a transposase enzyme non-covalently bound to a double stranded nucleic acid. For example, the complex can be a transposase enzyme preincubated with double-stranded transposon DNA under conditions that support non-covalent complex formation. Double-stranded transposon DNA can include, without limitation, Tn5 DNA, a portion of Tn5 DNA, a transposon end composition, a mixture of transposon end compositions or other double-stranded DNAs capable of interacting with a transposase such as the hyperactive Tn5 transposase.

The term “transposon end” (TE) refers to a double-stranded nucleic acid, e.g., a double-stranded DNA that exhibits only the nucleotide sequences (the “transposon end sequences”) that are necessary to form the complex with the transposase or integrase enzyme that is functional in an in vitro transposition reaction. In some embodiments, a transposon end is capable of forming a functional complex with the transposase in a transposition reaction. As non-limiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US Pat. Publ. No. 2010/0120098, the content of which is incorporated herein by reference in its entirety. Transposon ends can include any nucleic acid or nucleic acid analogue suitable for forming a functional complex with the transposase or integrase enzyme in an in vitro transposition reaction. For example, the transposon end can include DNA, RNA, modified bases, non-natural bases, modified backbone, and can include nicks in one or both strands. Although the term “DNA” is sometimes used in the present disclosure in connection with the composition of transposon ends, it should be understood that any suitable nucleic acid or nucleic acid analogue can be utilized in a transposon end.

As used herein, the terms “address,” “tag,” or “index,” when used in reference to a nucleotide sequence is intended to mean a unique nucleotide sequence that is distinguishable from other indices as well as from other nucleotide sequences within polynucleotides contained within a sample. A nucleotide “address,” “tag,” or “index” can be a random or a specifically designed nucleotide sequence. An “address,” “tag,” or “index” can be of any desired sequence length so long as it is of sufficient length to be unique nucleotide sequence within a plurality of indices in a population and/or within a plurality of polynucleotides that are being analyzed or interrogated. A nucleotide “address,” “tag,” or “index” of the disclosure is useful, for example, to be attached to a target polynucleotide to tag or mark a particular species for identifying all members of the tagged species within a population. Accordingly, an index is useful as a barcode where different members of the same molecular species can contain the same index and where different species within a population of different polynucleotides can have different indices. In a particular embodiment, the “address,” “tag,” or “index” comprises 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, or 100 nucleotides, or a range of nucleotides that includes or is between any two of the foregoing numbers (e.g., 5 to 50 nucleotides, 10 to 30 nucleotides, 15 to 25 nucleotides, etc.).

As used herein, a “spatial address,” “spatial tag” or “spatial index,” when used in reference to a nucleotide sequence, means an address, tag or index encoding spatial information related to the region or location of origin of an addressed, tagged, or indexed nucleic acid in a tissue sample. In a particular embodiment, the “spatial address,” “spatial tag” or “spatial index,” comprises 7, 8, 9, 10, 11, 12, 13, 14, 15, 16, 17, 18, 19, 20, 21, 22, 23, 24, 25, 30, 35, 40, 45, 50, 60, 70, 80, 90, or 100 nucleotides, or a range of nucleotides that includes or is between any two of the foregoing numbers (e.g., 8 to 50 nucleotides, 10 to 30 nucleotides, 15 to 25 nucleotides, etc.).

As used herein, the term “substrate” is intended to mean a solid support. The term includes any material that can serve as a solid or semi-solid foundation for creation of features such as wells for the deposition of biopolymers, including nucleic acids, polypeptide and/or other polymers. A substrate as provided herein is modified, for example, or can be modified to accommodate attachment of biopolymers by a variety of methods well known to those skilled in the art. Exemplary types of substrate materials include glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, a variety of polymers other than those exemplified above (e.g., cyclic olefin copolymers, polyacrylamide, cyclic olefin polymers, etc.), and multiwell microtiter plates. Specific types of exemplary plastics include acrylics, polystyrene, copolymers of styrene and other materials, polypropylene, polyethylene, polybutylene, polyurethanes and Teflon™. Specific types of exemplary silica-based materials include silicon and various forms of modified silicon. In a particular embodiment, a “substate” as used herein includes, but is not limited to beads, a microarray, a plate, a multiwell plate, or a flowcell (e.g., a nonpatterned flowcell, or a pattered flowcell). The substrate can comprise a planar surface, or comprise a non-planar (e.g., convex or concave) surface. Those skilled in the art will know or understand that the composition and geometry of a substrate as provided herein can vary depending on the intended use and preferences of the user. In some embodiments, the substrate may be patterned. For example, the substrate may be patterned with nanowells. Therefore, although planar substrates such as slides, chips or wafers are exemplified herein in reference to microarrays for illustration, given the teachings and guidance provided herein, those skilled in the art will understand that a wide variety of other substrates exemplified herein or well known in the art also can be used in the methods and/or compositions herein.

In a certain embodiment, a “substrate” disclosed herein may further comprises islands or clusters of immobilized capture agents or capture oligos. The islands or clusters can be generated on the surface of a substrate (e.g., a flowcell) by using bridge amplification. In such a case, the substrate comprises a plurality immobilized capture oligos on the surface of the substrate, which bind with complementary adapter regions presents on nearby primers or oligos to form bridge-like structures; these bridge-like structures are then extended using a polymerase enzyme, generating a double stranded molecule, that is then denatured to leave a single-stranded capture oligo anchored to the substrate. After multiple iterations of the foregoing process, islands or clusters of immobilized capture oligos are created. An example of the foregoing process that can be used with the methods and compositions disclosed herein can be found in WO 2022/015913 A1, which is incorporated herein by reference in-full. In a particular embodiment, the nearby primers or oligos are attached to the substrate (e.g., a flowcell) by a selectively cleavable linker. Each island or cluster may be roughly circular or oval in shape. Each island or cluster may have an average diameter of 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1050 nm, 1100 nm, 1200 nm, or a range that includes or is in between any two of the forgoing diameters. In a further embodiment, the surface of the substrate (e.g., a flowcell) comprises per 1 mm² of surface area 0.3, 0.4, 0.5, 0.6. 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, or 2.5 million clusters, or range including or between any two of the forgoing numbers. In a particular embodiment, a “substrate” as disclosed herein comprises islands or clusters of immobilized capture oligos comprising adapter sequence(s), a spatial address sequence, an optional sequence primer site, and a capture moiety for a targeted analyte. In yet a further embodiment, each cluster or island on the substrate (e.g., a flowcell) comprises capture oligos that have a unique spatial address sequence, so the x,y location of each cluster or island can be identified. In such a case, the x,y location of each cluster or island can be determined by decoding the spatial address sequence. Methods to decode the spatial address sequence include, but are not limited, the decoding-by-hybridization or the decoding-by-sequencing methods disclosed herein.

In some embodiments, the substrate is an ordered substrate. An “ordered substrate” refers to an arrangement of different regions in or on an exposed layer of a substrate, where each region comprises features (e.g., nanowells) that have an assigned x,y spatial address, or an x,y spatial address that can be readily determined. An “ordered substrate” may have a specific pattern of features. In some embodiments, the pattern can be a repeating arrangement of features and/or interstitial regions. In a certain embodiment, the surface(s) of an “ordered substrate” can be patterned with spatial address sequences. Exemplary patterned substrate that can be used in the methods and compositions set forth herein are described in U.S. Ser. No. 13/661,524 or US Pat. App. Publ. No. 2012/0316086 A1, each of which is incorporated herein by reference. In a particular embodiment, the features of an ordered substrate can comprise immobilized oligos, or islands or clusters of immobilized oligos. In such an embodiment, the location of the islands or clusters of immobilized capture oligos can be readily be determined without having to decode the spatial address sequence of immobilized oligos. Accordingly, immobilized oligos having a unique spatial address sequence is optional for an “ordered substrate.” Examples of “ordered substrates” include, but are not limited to, patterned flowcells, beadchip arrays, and microarrays.

As used herein, the term “interstitial region” refers to an area in a substrate or on a surface that separates other areas of the substrate or surface. For example, an interstitial region can separate one feature of an array from another feature of the array. The two regions that are separated from each other can be discrete, lacking contact with each other. In another example, an interstitial region can separate a first portion of a feature from a second portion of a feature. The separation provided by an interstitial region can be partial or full separation. Interstitial regions will typically have a surface material that differs from the surface material of the features on the surface. For example, features of an array can have an amount or concentration of capture agents or capture oligos that exceeds the amount or concentration present at the interstitial regions. In some embodiments the capture agents or primers may not be present at the interstitial regions.

In some embodiments, the substrate includes an array of wells or depressions in a surface. This may be fabricated as is generally known in the art using a variety of techniques, including, but not limited to, photolithography, stamping techniques, molding techniques and micro-etching techniques. As will be appreciated by those in the art, the technique used will depend on the composition and shape of the array substrate.

The features of a patterned substrate or an ordered substrate can be wells in an array of wells (e.g., microwells or nanowells) on glass, silicon, plastic or other suitable solid supports with patterned, covalently-linked gel such as poly(N-(5-azidoacetamidylpentyl)acrylamide-coacrylamide) (PAZAM, see, for example, U.S. Prov. Pat. App. Ser. No. 61/753,833, which is incorporated herein by reference). The process creates gel pads used for sequencing that can be stable over sequencing runs with a large number of cycles. The covalent linking of the polymer to the wells is helpful for maintaining the gel in the structured features throughout the lifetime of the structured substrate during a variety of uses. However, in many embodiments, the gel need not be covalently linked to the wells. For example, in some conditions silane free acrylamide (SFA, see, for example, U.S. Pat. App. Pub. No. 2011/0059865 A1, which is incorporated herein by reference) which is not covalently attached to any part of the structured substrate, can be used as the gel material.

In particular embodiments, a patterned substrate or ordered substrate can be made by patterning a solid support material with wells (e.g., microwells or nanowells), coating the patterned support with a gel material (e.g., PAZAM, SFA or chemically modified variants thereof, such as the azidolyzed version of SFA (azido-SFA)) and polishing the gel coated support, for example via chemical or mechanical polishing, thereby retaining gel in the wells but removing or inactivating substantially all of the gel from the interstitial regions on the surface of the structured substrate between the wells. Primer nucleic acids can be attached to gel material. A solution of target nucleic acids (e.g., a fragmented human genome) can then be contacted with the polished substrate such that individual target nucleic acids will seed individual wells via interactions with primers attached to the gel material; however, the target nucleic acids will not occupy the interstitial regions due to absence or inactivity of the gel material. Amplification of the target nucleic acids will be confined to the wells since absence or inactivity of gel in the interstitial regions prevents outward migration of the growing nucleic acid colony. The process is conveniently manufacturable, being scalable and utilizing conventional micro- or nano-fabrication methods. A patterned substrate or ordered substrate can include, for example, wells etched into a slide or chip.

The pattern of the etchings and geometry of the wells can take on a variety of different shapes and sizes so long as such features are physically or functionally separable from each other. Particularly useful substrates having such structural features are patterned substrates that can select the size of solid support particles such as microspheres. An exemplary patterned substrate having these characteristics is the etched substrate used in connection with BeadArray technology (Illumina, Inc., San Diego, Calif). Further examples, are described in U.S. Pat. No. 6,770,441, which is incorporated herein by reference.

In some embodiments, a substrate disclosed herein is a flowcell. The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference. A flowcell can be “a nonpatterned flowcell”, where the surface(s) of the flowcell comprises randomly or semi-randomly arranged features (e.g., areas comprising clusters or islands of oligos). Alternatively, the flowcell can be a “patterned flowcell,” where the flowcell comprises features (e.g., nanowells) at fixed locations across the surface(s) of the flowcell. The features of a “patterned flowcell” can further comprise immobilized oligos, or clusters or islands of immobilized oligos A “patterned flowcell” can be an “ordered substrate” in that the features of the patterned flowcell have an assigned x,y spatial address, or an x,y spatial address that can be readily determined.

As used herein, the term “immobilized” when used in reference to a nucleic acid is intended to mean direct or indirect attachment to a substrate or a feature of a substrate via covalent or non-covalent bond(s). In certain embodiments, covalent attachment can be used, but all that is required is that the nucleic acids remain stationary or attached to a support under conditions in which it is intended to use the support, for example, in applications requiring nucleic acid amplification and/or sequencing. Oligonucleotides to be used as capture primers or amplification primers can be immobilized such that a 3′-end is available for enzymatic extension and at least a portion of the sequence is capable of hybridizing to a complementary sequence. Immobilization can occur via hybridization to a surface attached oligonucleotide, in which case the immobilized oligonucleotide or polynucleotide can be in the 3′-5′ orientation. Alternatively, immobilization of oligos can comprise use of a selectively cleavable linker. Examples of selectively cleavable linkers include, but are not limited to, biotin-based molecules (e.g., desthiobiotin molecule(s) (ddBio)), PC Linker, and a recognition site for a rare-cutter enzyme. Typically, the selectively cleavable linker can be cleaved by heating, competitive binding, pH change, chemical cleavage, enzymatic cleavage and/or photo-cleavage. Cleaving the selectively cleavable linker results in the release the nucleic acid, or a portion thereof, from the substrate or feature of the substrate.

Certain embodiments may make use of an inert substrate or matrix (e.g., glass slides, polymer beads etc.) that has been functionalized, for example by application of a layer or coating of an intermediate material comprising reactive groups which permit covalent attachment to biomolecules, such as polynucleotides. Examples of such substrates include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (e.g., polynucleotides) may be directly covalently attached to the intermediate material (e.g., the hydrogel) but the intermediate material may itself be non-covalently attached to the substrate or matrix (e.g., the glass substrate). The term “covalent attachment to a substrate” is to be interpreted accordingly as encompassing this type of arrangement.

Exemplary covalent linkages include, for example, those that result from the use of click chemistry techniques. Exemplary non-covalent linkages include, but are not limited to, non-specific interactions (e.g., hydrogen bonding, ionic bonding, van der Waals interactions etc.) or specific interactions (e.g., affinity interactions, receptor-ligand interactions, antibody epitope interactions, avidin-biotin interactions, streptavidin-biotin interactions, lectin carbohydrate interactions, etc.). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “plurality” is intended to mean a population of two or more different members. Pluralities can range in size from small, medium, large, to very large. The size of small plurality can range, for example, from a few members to tens of members. Medium sized pluralities can range, for example, from tens of members to about 100 members or hundreds of members. Large pluralities can range, for example, from about hundreds of members to about 1000 members, to thousands of members and up to tens of thousands of members. Very large pluralities can range, for example, from tens of thousands of members to about hundreds of thousands, a million, millions, tens of millions and up to or greater than hundreds of millions of members. Therefore, a plurality can range in size from two to well over one hundred million members as well as all sizes, as measured by the number of members, in between and greater than the above exemplary ranges. An exemplary number of features within a microarray includes a plurality of about 500,000 or more discrete features within 1.28 cm². Exemplary nucleic acid pluralities include, for example, populations of about 1×10⁵, 5×10⁵ and 1×10⁶ or more different nucleic acid species. Accordingly, the definition of the term is intended to include all integer values greater than two. An upper limit of a plurality can be set, for example, by the theoretical diversity of nucleotide sequences in a nucleic acid sample.

As used herein, the term “tissue sample” refers to a piece of tissue that has been obtained from a subject, fixed, sectioned, and mounted on a planar surface, e.g., a microscope slide. The tissue sample can be a formalin-fixed paraffin-embedded (FFPE) tissue sample or a fresh tissue sample or a frozen tissue sample, etc. The methods disclosed herein may be performed before or after staining the tissue sample. For example, following hematoxylin and eosin staining, a tissue sample may be spatially analyzed in accordance with the methods as provided herein. A method may include analyzing the histology of the sample (e.g., using hematoxylin and eosin staining) and then spatially analyzing the tissue.

As used herein, the term “formalin-fixed paraffin embedded (FFPE) tissue section” refers to a piece of tissue, e.g., a biopsy that has been obtained from a subject, fixed in formaldehyde (e.g., 3%-5% formaldehyde in phosphate buffered saline) or Bouin solution, embedded in wax, cut into thin sections, and then mounted on a planar surface, e.g., a microscope slide.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

The emerging field of spatial proteogenomics is being driven by the development of new technologies that allow the mapping of single cell -omes to their spatial locations in a tissue. Spatial genomic technologies capture the ‘where’ of biological information. Products today use spotted arrays or physically isolated droplets to capture nucleic acid information from cell populations or histological samples. The resolution today is limited by the density of features that can be used to identify x,y positions of transcripts and proteins. Additionally, the current technologies cannot be used for multi-omic applications.

Described herein are improved methods and compositions for spatial omics applications that preserve spatial information related to the origin of analytes in the tissue. Examples of spatial omics applications include, but are not limited to, spatial genomic applications, spatial proteomic applications; spatial transcriptomic applications; spatial agrigenomic applications; spatial epigenomics s applications; spatial phenomic applications; spatial ligandomic applications; and spatial multiomic applications (e.g., trinscriptomic and genomic applications).

The present disclosure is based, in part, on the realization that information related to the spatial origin of analytes in a tissue sample can be correlated with the x,y position in an array, by using probes attached to an array or to an intermediate substrate, like a bead, the probes comprising a “spatial address” and a capture moiety for a targeted analyte from the tissue. The “spatial address” can be decoded by using various technologies including, but not limited to, decoding-by-hybridization and by decoding-by-sequencing. In a particular embodiment, the decoding method is the decoding-by-hybridization method taught in Gunderson et al. (Genome Res. 14(5):870-874) (2004)). The probe's capture moiety can then bind or hybridize with a target analyte from the tissue sample ex situ. Alternatively, the spatially addressed probes may be first detached from the array or the intermediate substrate (e.g., bead) and then bound to target analytes in the tissue sample in situ. The oligonucleotides bound to different analytes in the tissue sample can be distinguished based on their spatial address and can be mapped onto their regions of origin in the tissue sample, thereby providing spatial omics information. Examples of analytes in a tissue sample include genomic DNA, methylated DNA, specific methylated DNA sequences, messenger RNA (mRNA), polyA mRNA, fragmented mRNA, fragmented DNA, mitochondrial DNA, viral RNA, microRNA, in situ synthesized PCR products, RNA/DNA hybrids, lipids, carbohydrates, proteins, glycoproteins, lipoproteins, phosphoproteins, specific phosphorylated or acetylated variant of a protein, or viral coat proteins.

The present disclosure is further based, in part, on the realization that spatially addressable probes can be attached to the surface of an array or an intermediate substrate (e.g., beads or nanoparticles) so that the probes bind to a target analyte in a sample for omic applications. In a particular embodiment, the spatially addressable probes attached to the surface of an array or the intermediate substrate comprise the same spatial address. In another embodiment, the spatially addressable probes are reversibly attached to the surface of an array of the intermediate substrate by using desthiobiotin molecule(s). In a further embodiment, the intermediate substrate is beads. In yet a further embodiment, the beads are placed into nanowells patterned on the surface of a substrate (e.g., a beadchip). In a further embodiment, thousands of probes are attached to beads, the spatially addressable probes all having the same spatial address sequence. n yet a further embodiment, the probes are reversibly attached to the beads using desthiobiotin molecule(s) (ddBio), which allows probe release with limited lateral diffusion under defined conditions. By use of such reversible detachment, the probes can diffuse to the targeted analytes (rather than the other way around), which may improve analyte capture and assay sensitivity.

The present disclosure is further based, in part, on the realization that multiple types of probes can be attached to an intermediate substrate (e.g., beads or nanoparticles) so that the different types of probes bind to different target analytes in a sample for multi-omic applications. In a particular embodiment, at least one of the types of probes attached to the intermediate substrate comprise a spatial address. In another embodiment, all of the probes attached to the intermediate substrate comprise the same spatial address. In yet another embodiment, the multiple types of probes attached to the intermediate substrate (e.g., beads or nanoparticles) differ by comprising different capture moieties. In certain embodiments, the capture moieties may differ by comprising the same type of nucleic acids but have different sequences. In such a case, the probes can be used, for example, in epigenomic applications. In other instances, the capture moieties may differ by comprising different types of nucleic acids, e.g., RNA and DNA. In such a case, these probes can be used, for example, in transcriptomics and genomics applications. In additional embodiments, the capture moieties may differ by comprising different types of biomolecules, e.g., DNA and proteins (e.g., antibodies). Examples of biomolecules that can be used as capture moieties, include, but are not limited to, nucleic acids, antibodies, aptamers, scFvs, antigen binding domains, proteins, peptides, receptors, haptens, etc. In such a case, these probes can be used, for example, in genomics and proteomics applications. In yet additional embodiments, the capture moieties may differ according to the following: comprising the same type of nucleic acids but have different sequences, comprise different types of nucleic acids, comprise different types of biomolecules, or any combination of the foregoing. In a further embodiment, the multiple probes are reversibly attached to beads. In yet a further embodiment, the beads are placed into nanowells patterned on the surface of a substrate (e.g., a beadchip). In a further embodiment, thousands of probes are attached to beads, the probes all having the same spatial address. In yet a further embodiment, thousands of probes are reversibly attached to beads, the probes all having the same spatial address sequence. In yet a further embodiment, the multiple types of probes are reversibly attached to the beads using desthiobiotin molecule(s) (ddBio), which allows probe release with limited lateral diffusion under defined conditions. By use of such reversible detachment, the multiple probes can diffuse to the targeted analytes (rather than the other way around), which may improve analyte capture and assay sensitivity.

In another embodiment, the disclosure provides for nanoparticles or beads which comprise the spatially addressable probes disclosed herein. In a particular embodiment, beads comprise the spatially addressable probes disclosed herein. In a further embodiment, the bead comprises streptavidin on the surface of the bead. In yet a further embodiment, the beads comprise a plurality of oligos bound to the bead via a linkage or a reversible linkage. Examples of reversible linkages include biotin molecule(s), such as ddBio molecules. The oligos bound the bead typically comprise an adaptor sequence, such as P5 sequence or a P7 sequence. As used herein a P5 sequence comprises a sequence defined by SEQ ID NO: 1 (AATGATACGGCGACCACCGA) and a P7 sequence comprises a sequence defined by SEQ ID NO: 2 (CAAGCAGAAGACGGCATACGA). In some embodiments, the P5 or P7 sequence can further include a spacer polynucleotide, which may be from 1 to 20, such as 1 to 15, or 1 to 10, nucleotides, such as 2, 3, 4, 5, 6, 7, 8, 9, or 10 nucleotides in length. In some embodiments, the spacer includes 10 nucleotides. In some embodiments, the spacer includes 10 nucleotides. In some embodiments, the spacer is a polyT spacer, such as a 10T spacer. Spacer nucleotides may be included at the 5′ ends of polynucleotides, which may be attached to a suitable support via a linkage with the 5′ end of the oligo. Attachment can be achieved through a sulfur-containing nucleophile, such as phosphorothioate, present at the 5′ end of the polynucleotide. In some embodiments, the oligos will include a polyT spacer and a 5′ phosphorothioate group. Thus, in some embodiments, the P5 sequence is 5′phosphorothioate-TTTTTTTTTTAATGATACGGCGACCACCGA-3′ (SEQ ID NO: 3), and in some embodiments, the P7 sequence is 5′phosphorothioate-TTTTTTTTTTCAAGCAGAAGACGGCATACGA-3′ (SEQ ID NO: 4). In certain embodiments, the oligos attached to the beads comprise an address sequence that allows for determining the x,y position of the oligo/bead when decoded. In further embodiments, the address sequence comprises 7, 8, 9, 10, 11, 12, 13, 14, 15, 16, 17, 18, 19, 20, 21, 22, 23, 24, 25, 30, 35, 40, 45, 50, 60, 70, 80, 90, or 100 nucleotides, or a range of nucleotides that includes or is between any two of the foregoing numbers (e.g., 8 to 50 nucleotides, 10 to 30 nucleotides, 15 to 25 nucleotides, etc.). In another embodiment, the oligos attached to the beads comprise a transposome hybridization region (Tsm hyb). In yet additional embodiments, the oligos comprise sequencing primer(s) site sequence(s). Examples of sequencing primer site sequences include sequences that are complementary to Read 1 (R1) and Read 2 (R2) sequencing primers from Illumina™. Examples of R1 sequencing primer site sequences include R1 SBS3
(long):

(SEQ ID NO: 5)
ACACTCTTTCCCTACACGACGCTCTTCCGATCT;

and R1 SBS3 (short): ACACTCTTTCCCTACACGAC (SEQ ID NO:6). Examples of R2 sequencing primer site sequences include R2 SBS12
(long):

(SEQ ID NO: 7)
GTGACTGGAGTTCAGACGTGTGCTCTTCCGATCT;

and R2 SBS12 (short): GTGACTGGAGTTCAGACGTGT (SEQ ID NO:8).

In further embodiments, the oligos may further comprise one or more linker sequences. In yet further embodiment, the oligos may further comprise one or more index sequences. In certain embodiments, the oligos may comprise one or more unique molecular identifier (UMI) sequences. Unique molecular identifiers (UMIs) are a type of molecular barcoding that provides error correction and increased accuracy during sequencing. These molecular barcodes are short sequences used to uniquely tag each molecule in a sample library. UMIs are used for a wide range of sequencing applications, many around PCR duplicates in DNA and cDNA. UMI deduplication is also useful for RNA-seq gene expression analysis and other quantitative sequencing methods. In a particular embodiment, the UMI comprises 6, 7, 8, 9, 10, 11, 12, 13, 14, 15, 16, 17, 18, 19, 20, 21, 22, 23, 24, 25, 26, 27, 28, 29, or 30 nucleotides, or a range of nucleotides that includes or is between any two of the foregoing numbers (e.g., 8 to 20, 8 to 16, etc.). As noted previously, the oligos comprise moieties or sequences that can bind with specificity to analytes from a biological sample (e.g., a tissue sample). As such, the oligos attached to the beads are spatially addressable probes for analytes from a biological sample. The moieties or sequences that can bind with specificity to analytes from a biological sample can be selected for a particular omic application. For example, the oligos can comprise an oligo d(T) sequence for transcriptomics or for assay (e.g., RNA-seq assays). Alternatively, the oligos can comprise sequences to bind with genomic DNA from a biological sample for genomic applications or for assays (e.g., ATAC-seq assays). As provided in the Examples presented herein, the beads can comprise multiple types of oligos that have different moieties or sequences so that the spatially addressable probes can bind specifically to two or more different types of analytes from a biological sample. The use of multi types of oligos is ideally suited for multi-omic or multiple assay applications.

In a certain embodiment, the disclosure provides methods and compositions for spatial omics that utilize beadchips with high feature densities. Beadchips, such as the Infinium beadchip developed by Illumina™, have feature densities that are 3000-fold more dense than other arrayed features taught in the field of spatial genomics (see Table 1).

TABLE 1
Feature density for various arrays

| | Area (μm²) | Features | Feature Density (Feature/mm²) |
|---|---|---|---|
| Visium | 42250000 | 5000 | 118 |
| SlideSeq | 14130000 | 70000 | 4954 |
| Infinium | 48750000 | 18200000 | 373333 |

As beadchips have an x,y format they are ideally suited for omic applications and systems. The beadchips can provide spatial multi-omics information by adding in probes that capture protein analytes of interests. For example, antibodies or aptamers which comprise an appended sequence can be used to bind to targeted protein analytes and then be attached to beads by hybridizing the appended sequence to a complementary capture sequence of the probe (e.g., see FIG. 2). The x,y position of the probe can be determined by decoding the spatial address sequence. Resolution of the beachchip arrays can be further increased with smaller beads, nanowells, imaging sensitivity, etc. The specific analytes (nucleic acid, protein) can be detected, as well as, the address sequence and other sequences can be decoded.

In a particular embodiment, the disclosure provides nanoparticles or beads which comprise spatially addressable probes

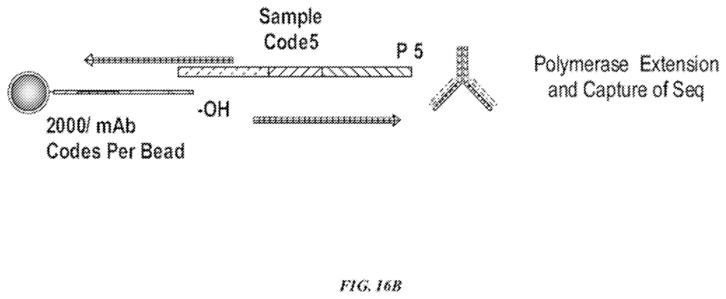

The spatially addressable probes may be contacted with tissue by placing the tissue directly on the surface comprising the probes (e.g., see FIG. 16A-B); placing the tissue on a substance, such as a filter or a gel or a thin buffer layer, separating the tissue from the spatially addressable probes such that the target nucleic acids may diffuse from the tissue, through the substance to the spatially addressable probes; placing the tissue on a substance such as a filter or a gel or a thin buffer layer separating the tissue from the spatially addressable probes such that the spatially addressable probes may diffuse from the surface comprising the spatially addressable probes, through the substance to the targets; extracting the targets from the tissue onto an intermediate substrate (e.g., a gel, filter, solid substrate, or combinations of the foregoing), which is then placed on the surface supporting the probes; and combinations of the foregoing. In each case, the technique is selected to substantially maintain information encoding the spatial orientation of the targets in the sample.

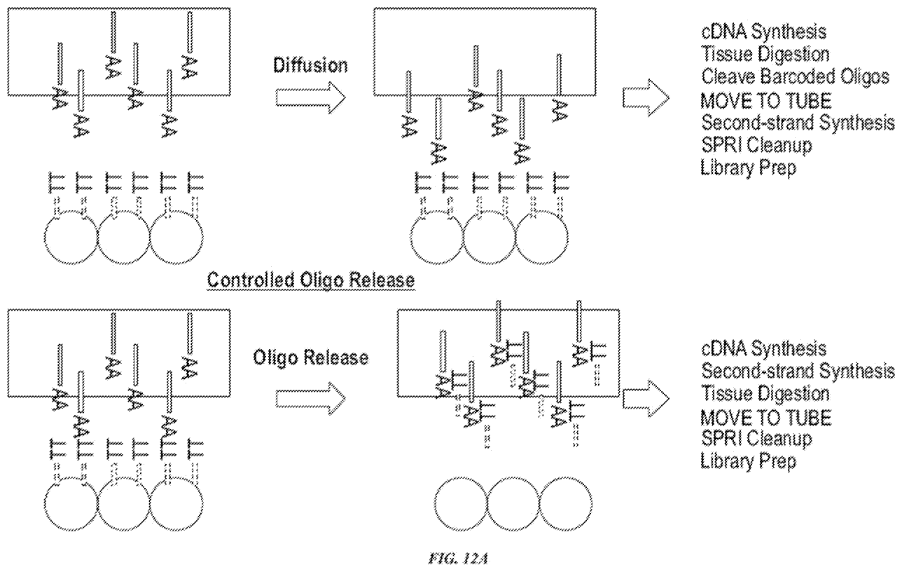

In another example, the tissue sample is contacted with the array and the spatially addressable probes on the array are released into the tissue sample for hybridization to the nucleic acids in the tissue sample (e.g., see FIG. 12A-C). The tissue sample may be placed directly on the surface comprising the spatially addressable probes or the tissue sample may be placed on a substance such as a filter or a gel or a thin buffer layer separating the tissue sample from the spatially addressable probes such that the released spatially addressable probes may diffuse from the array through the substance to the nucleic acid in the tissue sample. The probes may be anchored on the array using a releasable group or a selectively cleavable portion or linker (e.g., desthiobiotin molecule(s)). The spatially addressable probes may be released from the array using, for example, chemical cleavage, enzymatic cleavage or photo-cleavage. In another example, the spatially addressable probes may be printed onto the surface of the array and dried down. The spatially addressable probes may be released from the array by rehydration. In yet another example, the spatially addressable probes may be printed onto the array using a substance that dissolves in the presence of a certain treatment. The treatment to release the spatially addressable probes is then applied prior to the placement of the tissue sample onto the array.

In a particular embodiment, the tissue used in the compositions, methods and kits disclosed herein is fresh unprocessed tissue. In alternate embodiment, the tissue used in the compositions, methods and kits disclosed herein is processed prior to being used in the compositions, methods and kits of disclosure. Methods for processing the tissue can include the steps of fixing the tissue, embedding the tissue, staining the tissue, and sectioning the tissue. The tissue may be fixed using chemical fixatives or by fast freezing. Chemical fixatives can be coagulant fixatives, which remove water from tissues leading to coagulation and denaturalization of proteins, mostly in the extracellular matrix; and crosslinking fixatives form chemical bonds between molecules of the tissue. Examples of coagulant fixatives include, but are not limited to, ethanol, methanol, picric, acid, acetone, Clarke solution, Bouin and Camoy. Examples of crosslinking fixatives include, but are not limited to, formaldehyde and glutaraldehyde. In a particular embodiment, the tissue used in the compositions, methods and kits disclosed herein is not fixed with chemical fixatives. In a particular embodiment, the tissue used in the compositions, methods and kits disclosed herein is fixed by being fast frozen. The tissue used in the compositions, methods and kits disclosed herein may be processed herein by being embedded and sectioned. The embedding process typically entails the use of an embedding agent to embed the tissue. Examples of embedding agents, include but are not limited to, paraffin, celloidin, epoxy resin, acrylic resin, and optimal cutting temperature (OCT) compound. An OCT compound is a water-soluble blend of glycols and resins that provides a convenient specimen matrix for cryostat sectioning at temperatures of −10° C. and below. In a particular embodiment, the tissue used in the compositions, methods and kits disclosed herein is embedded in an OCT compound. Once embedded or fixed, the tissue is typically cut into very thin sections from 50 nanometers up to 100 μm using devices like an utracryotome, freezing microtome, or microtome. The tissue sections can be stained prior to being placed on an array disclosed herein. Alternatively, the tissue sections are not stained prior to being placed on an array disclosed herein. Staining is the process for coloring tissues by using dyes. It allows visualizing cells and extracellular matrix to be studied with light microscopes. A dye molecule has two domains: the chromogen provides the color and the auxochrome makes possible the binding to the tissue. Auxochromes are chemically variable: can be ionizable, can react with metallic ions (they are then called mordant), or can react with tissular molecules. Most dyes are soluble in water. In a particular embodiment, the tissue sections are stained with hematoxylin and eosin. Hematoxylin is used to visualize negatively charged DNA. Eosin is used to visualize positively charged groups such as amino groups. Accordingly, the compositions, methods and kits of the disclosure can provide spatial omics information that can be directly correlated to stained structures in the tissue section.

For use in the spatial omic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more spatially addressable probes disclosed herein, optionally in a composition or in combination with another agent (e.g., an array, a beadchip) as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use with the spatially addressable probes described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific spatial omic applications. The label can also indicate directions for use of the contents, such as in the methods described herein.

The disclosure further provides that the methods and compositions described herein can be further defined by the following aspects (aspects 1 to 92):

1. A spatial genomics Assay for Transposase-Accessible Chromatin with high-throughput sequencing (ATAC-Seq) method, comprising
(A) loading particles into wells of a substrate, wherein the particles comprise oligos that are attached to the particles through a selectively cleavable linker, the oligos comprising an adapter sequence, a spatial address sequence, and a transposome hybridization region;
(B) decoding the spatial address sequences of the oligos to determine the x,y position of the particles;
(C) placing tissue on top of the particles in the array, and lysing the cell membranes to access chromatin regions in the tissue;
(D) tagmentating the chromatin regions with a transposome complex to form tagmented fragments, and removing the transposome complex;
(E) permeabilizing the tissue to allow diffusion of the tagmented fragments to the particles in the array;
(F) capturing the tagmented fragments to the particles by hybridizing the tagmented fragments to the transposome hybridization region of the oligos;
(G) processing the captured tagmented fragments to make particle-bound genomic library constructs for sequencing;
(H) releasing the genomic library constructs from the particles by selectively cleaving the linker;
(I) obtaining spatial genomics information of the tissue by sequencing the genomic library constructs using a sequencer and mapping the sequencing reads with x,y positions of the particles,
optionally, wherein the particles are microparticles or nanoparticles; and/or
optionally, wherein the substrate is selected from a microarray, a beadchip, a multiwell plate, or a flowcell; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the spatial address sequence comprises 8 to 50 nucleotides, yet more optionally wherein the spatial address sequence comprises 10 to 30 nucleotides; and/or
optionally, wherein the transposome hybridization region comprises a sequence that is complementary to tagmented ends of polynucleotides generated by a transposome complex; and/or
optionally, the selectively cleavable linker is selected from biotin-based molecules, PC Linker, and a recognition site for a rare-cutter enzyme, more optionally, the selectively cleavable linker comprises desthiobiotin molecule(s) (ddBio); and/or
optionally, wherein the selectively cleavable linker can be cleaved by heating, competitive binding, pH change, chemical cleavage, enzymatic cleavage and/or photo-cleavage, more optionally, wherein the selectively cleavable linker can be cleaved by competitive binding and heat, more optionally, the selectively cleavable linker is cleaved by adding biotin and heating.

2. The spatial genomics ATAC-Seq method of aspect 1, wherein the nanoparticles are beads, optionally, wherein the beads are coated with streptavidin, more optionally, wherein the nanoparticles are streptavidin coated magnetic beads.

3. The spatial genomics ATAC-Seq method of aspect 1 or aspect 2, wherein the transposome complex is a TN5-based transposome complex.

4. The spatial genomics ATAC-Seq method of any one of the preceding aspects, wherein the spatial address sequences are decoded by using a decoding-by-hybridization method.

5. The spatial genomics ATAC-Seq method of any one of the preceding aspects, wherein the cell membranes are lysed by using a mild detergent, optionally, wherein the mild detergent is selected from NP40, Tween 20, Bile Salts, Triton X100, and (3-((3-cholamidopropyl)dimethylammonio)-1-propanesulfonate) (CHAPS).

6. The spatial genomics ATAC-Seq method of any one of the preceding aspects, wherein the tissue is permeabilized by using a detergent and Proteinase K, optionally wherein the detergent is Tween 20 and/or Triton X100.

7. The spatial genomics ATAC-Seq method of any one of the preceding aspects, where the captured tagmented fragments are processed by using top strand ligation, hybridization to Y-shaped adapters, and gap-filling ligations steps,
optionally wherein the top strand ligation utilizes a DNA ligase, and/or,
optionally wherein the gap-filling ligation step comprises use of a DNA polymerase lacking strand-displacement activity, dNTPs, and a DNA ligase, more optionally, the DNA polymerase lacking strand-displacement activity is selected from a T4-based polymerase, a T7-based polymerase, a Pfu-based polymerase, or a Taq-based polymerase, and/or
optionally, wherein the Y-shaped adapters are noncomplementary at their ends to prevent their self-ligation and thus form a Y shape after annealing to the oligos.

8. The spatial genomics ATAC-Seq method of any one of the preceding aspects, wherein prior to sequencing the library constructs are amplified using PCR.

9. The spatial genomics ATAC-Seq method of any one of the preceding aspects, wherein the sequencer utilizes sequencing by synthesis technology.

10. The spatial genomics ATAC-Seq method of any one of the preceding aspects, wherein the hybridization region of the transposome complex is initially blocked by use of an accessory oligo that is then removed in step (E).

11. A spatial multi-omics Assay for Transposase-Accessible Chromatin with high-throughput sequencing (ATAC-Seq) and RNA-Seq method, comprising:
(A) loading particles into wells of an array, wherein the particles comprise two sets of oligos that attached to the particles through a selectively cleavable linker, the first set of oligos comprising a first adapter sequence, a spatial address sequence, and a transposome hybridization region, the second set of oligos comprising the first adapter sequence, a sequencing primer site sequence, a spatial address sequence, a unique molecular identifier (UMI) sequence, and an oligo(dT) sequence;
(B) decoding the spatial address sequences of the oligos to determine the x,y position of the particles;
(C) placing tissue on top of the particles in the array, and lysing the cell membranes to access poly-A RNA transcripts and chromatin regions in the tissue;
(D) tagmentating the chromatin regions with a first transposome complex to form tagmented fragments, and removing the first transposome complex;
(E) permeabilizing the tissue to allow diffusion of the tagmented fragments and poly-A RNA transcripts to the particles in the array;
(F) capturing the tagmented fragments and poly-A RNA transcripts to the nanoparticles by (i) hybridizing the tagmented fragments to the transposome hybridization region of the first set of oligos, and (ii) hybridizing the poly-A RNA transcripts to the oligo(dT) sequence of the second set of oligos;
(G′) processing the first set of oligos comprising captured tagmented fragments to make particle bound genomic library constructs for sequencing;
(G″) reverse transcribing the second set of oligos comprising captured poly-A RNA transcripts in the presence of a single stranded template switch oligo to make nanoparticle bound cDNA library constructs for sequencing;
(H) releasing the genomic library constructs and the cDNA library constructs from the particles by selectively cleaving the linker;
(I) amplifying the genomic library constructs and cDNA library constructs using PCR and splitting the amplified products into two portions comprising both amplified constructs;
(J) amplifying a first portion of the amplified constructs with a first adapter primer and a second adapter primer to form an ATAC-Seq library, wherein the first adapter primer and the second adapter primer bind to adapter sequences, and wherein the first adapter primer and/or the second adapter primer further comprises an index sequence and/or a sequencing primer site sequence;
(J′) tagmentating a second portion of the amplified constructs with a primer comprising the sequence primer sequence and TSM and then amplifying the tagmented amplified library constructs using PCR with the first adapter primer comprising a sequencing primer site sequence and the second adapter primer comprising an index sequence and a sequencing primer site sequence to form an RNA-Seq library, wherein the first adapter primer and the second adapter primer have difference sequence primer sequences; and
(K) obtaining multi-omics information by sequencing the ATAC-Seq library and RNA-Seq library using a sequencer and mapping the sequencing reads with x,y positions of the particles.
optionally, wherein the particles are microparticles or nanoparticles; and/or
optionally, wherein the first adapter sequence comprises a P5 adapter sequence and the second adapter sequence comprises a P7 adapter sequence, or vice versa, more optionally, wherein the first adapter sequence comprises the sequence of SEQ ID NO:1 and the second adapter sequence comprises the sequence SEQ ID NO:2, or vice versa; and/or
optionally, wherein the first adapter primer comprises a complementary P5 adapter sequence and the second adapter primer comprises a complementary P7 adapter sequence, or vice versa, more optionally, wherein the first adapter primer comprises a sequence complementary to SEQ ID NO:1 and the second adapter primer comprises a sequence complementary to SEQ ID NO:2, or vice versa; and/or
optionally, wherein the UMI sequence comprises 8 to 20 nucleotides, more optionally wherein the UMI sequence comprises 8 to 16 nucleotides; and/or
optionally, wherein the spatial address sequence comprises 8 to 50 nucleotides, yet more optionally wherein the spatial address sequence comprises 10 to 30 nucleotides; and/or
optionally, wherein the transposome hybridization region comprises a sequence that is complementary to tagmented ends of polynucleotides generated by a transposome complex; and/or
optionally, wherein the sequencing primer site sequence of the first adapter primer comprises a Read 1 (R1) sequencing primer site sequence, more optionally, the second adapter sequence comprises a R1 sequencing primer site sequence of SEQ ID NO:5 or SEQ ID NO:6; and/or
optionally, wherein the sequencing primer site sequence of the second adapter sequence comprises a Read 2 (R2) sequencing primer site sequence, more optionally the second adapter sequence comprises a R2 sequencing primer site sequence of SEQ ID NO:7 or SEQ ID NO:8; and/or
optionally, wherein the index sequence comprises 6 to 10 nucleotides; and/or
optionally, the selectively cleavable linker is selected from biotin-based molecules, PC Linker, and a recognition site for a rare-cutter enzyme, more optionally, the selectively cleavable linker comprises desthiobiotin molecule(s) (ddBio); and/or
optionally, wherein the selectively cleavable linker can be cleaved by heating, competitive binding, pH change, chemical cleavage, enzymatic cleavage and/or photo-cleavage, more optionally, wherein the selectively cleavable linker can be cleaved by competitive binding and heat, more optionally, the selectively cleavable linker is cleaved by adding biotin and heating.

12. The spatial multi-omics ATAC-Seq and RNA-Seq method of aspect 11, wherein the particles are beads, optionally, wherein the beads are coated with streptavidin, more optionally, wherein the nanoparticles are streptavidin coated magnetic beads.

13. The spatial multi-omics ATAC-Seq and RNA-Seq method of aspect 11 or aspect 12, wherein the transposome complex is a TN5 transposome complex.

14. The spatial multi-omics ATAC-Seq and RNA-Seq method of any one of aspects 11 to 13, wherein the spatial address sequences are decoded by using a decoding-by-hybridization method.

15. The spatial multi-omics ATAC-Seq and RNA-Seq method of any one of aspects 11 to 14, wherein the cell membranes are lysed by using a mild detergent, optionally, wherein the cell membranes are lysed using a mild detergent selected from NP40, Tween 20, Bile Salts, Triton X100, and (3-((3-cholamidopropyl)dimethylammonio)-1-propanesulfonate) (CHAPS).

16. The spatial multi-omics ATAC-Seq and RNA-Seq method of any one of aspects 11 to 15, wherein the tissue is permeabilized by using a detergent and Proteinase K, optionally wherein the detergent is Tween 20 and/or Triton X100.

17. The spatial multi-omics ATAC-Seq and RNA-Seq method of any one of aspects 11 to 16, where the captured tagmented fragments are processed by using top strand ligation, hybridization to Y-shaped adaptors, and gap-filling ligations steps,
optionally, wherein the top strand ligation utilizes a DNA ligase, and/or,
optionally, wherein the gap-filling ligation step comprises use of a DNA polymerase lacking strand-displacement activity, dNTPs, and a DNA ligase, more optionally, the DNA polymerase lacking strand-displacement activity is selected from a T4-based polymerase, a T7-based polymerase, a Pfu-based polymerase, or a Taq-based polymerase, and/or
optionally, wherein the Y-shaped adapters e are noncomplementary at their ends to prevent their self-ligation and thus form a Y shape after annealing to the oligos.

18. The spatial multi-omics ATAC-Seq and RNA-Seq method of any one of aspects 11 to 17, wherein prior to sequencing the library constructs are amplified using PCR.

19. The spatial multi-omics ATAC-Seq and RNA-Seq method of any one of aspects 11 to 18, wherein the sequencer utilizes sequencing by synthesis technology.

20. The spatial multi-omics ATAC-Seq and RNA-Seq method of any one of aspects 11 to 19, wherein the hybridization region of the transposome complex is initially blocked by use of an accessory oligo that is then removed in step (E).

21. The spatial multi-omics ATAC-Seq and RNA-Seq method of any one of aspects 11 to 20, wherein steps (G) and (G′) are performed sequentially or simultaneously.

22. The spatial multi-omics ATAC-Seq and RNA-Seq method of any one of aspects 11 to 21, wherein steps (J) and (J′) are performed sequentially or simultaneously.

23. The spatial multi-omics ATAC-Seq and RNA-Seq method of any one of aspects 11 to 22, wherein the oligo d(T) sequence comprises 16 to 20 nucleotides.

24. A spatial transcriptomics method using a beadchip, comprising:
(A) loading beads into wells of a beadchip, wherein the beads comprise oligos comprising an adapter sequence, a spatial address sequence, an optional sequence primer site, and an oligo(dT) sequence;
(B) decoding the spatial address sequences of the oligos to determine the x,y position of the beads;
(C) placing tissue on top of the beadchip, and lysing the cell membranes to access poly-A RNA transcripts in the tissue;
(D) capturing the poly-A RNA transcripts to the beads;
(E) reverse transcribing the oligos comprising captured poly-A RNA transcripts: (i) in the presence of a single stranded template switch oligo to make a bead bound cDNA library constructs for sequencing, or (ii) further performing a single strand ligation reaction with a single stranded oligo to make a bead bound cDNA library constructs for sequencing;
(F) amplifying the bead bound cDNA library constructs using PCR and an adapter sequence primer;
(G) obtaining transcriptomics information by sequencing the amplified cDNA library constructs using a sequencer and mapping the sequencing reads with x,y positions of the beads in the beadchip,
optionally, wherein the wells of the beadchip are nanowells; and/or
optionally, wherein the nanoparticles are streptavidin coated beads, more optionally, wherein the nanoparticles are streptavidin coated magnetic beads; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the adapter primer comprises a complementary P5 adapter sequence or a complementary P7 adapter sequence, more optionally, wherein the first adapter primer comprises a sequence complementary to SEQ ID NO:1 or a sequence complementary to SEQ ID NO:2, and/or
optionally, wherein the sequencing primer site sequence comprises a Read 1 (R1) sequencing primer site sequence, more optionally, wherein the sequencing primer site sequence comprises a R1 sequencing primer site sequence of SEQ ID NO:5 or SEQ ID NO:6; and/or
optionally, wherein the sequencing primer site sequence comprises a Read 2 (R2) sequencing primer site sequence, more optionally, wherein the sequencing primer site sequence comprises a R2 sequencing primer site sequence of SEQ ID NO:7 or SEQ ID NO:8; and/or
optionally, wherein the spatial address sequence comprises 8 to 50 nucleotides, yet more optionally wherein the spatial address sequence comprises 10 to 30 nucleotides; and/or
optionally, the tissue is placed in removable incubation chamber(s) that is affixed to the top of the beadchip, wherein different sections of the beadchip comprise removable incubation chamber(s) and/or different sections of the beadchip comprise separate removable incubation chamber(s); and/or
optionally, wherein the cell membranes are lysed by using a mild detergent,
   optionally, wherein the cell membranes are lysed using a mild detergent selected from NP40, Tween 20, Bile Salts, Triton X100, and (3-((3-cholamidopropyl)dimethylammonio)-1-propanesulfonate) (CHAPS).

25. The spatial transcriptomics method of aspect 24, wherein the beadchip has a feature density (feature/mm²) of greater than 300000.

26. The spatial transcriptomics method of aspect 25, wherein the beads further comprise lanthanide nanophosphor labels.

27. A spatial multi-omics method to detect targeted RNA and proteins, comprising:
(A) loading multiple sets of lanthanide nanophosphor labeled beads into an array, wherein a first set of beads comprise oligos comprising an adapter sequence, a spatial address sequence, an optional sequence primer site sequence, and an oligo(dT) sequence, and a second set of beads that comprise oligos comprising a spatial address sequence, a UMI sequence, and a capture sequence;
(B) decoding the spatial address sequences of the oligos to determine their x,y position in the array;
(C) placing tissue on top of the beads, and lysing the cell membranes to access poly-A RNA transcripts and proteins in the tissue;
(D) adding antibodies comprising a barcode nucleotide sequence which bind with specificity to protein targets;
(E) capturing the poly-A RNA transcripts to the first set of beads;
(E′) capturing the antibodies to the second set of beads by hybridizing the barcode nucleotide sequence of the antibodies to the capture sequence;
(F) reverse transcribing the oligos comprising captured poly-A RNA transcripts with oNTPS;
(G) adding a primer which binds to oligos on the sets of beads and extending the primers with oNTPS;
(H) adding a dsDNA dye that binds to the extended primer sequences; and
(I) detecting targeted RNA and proteins by imaging and decoding the sets of beads,
optionally, wherein the nanoparticles are streptavidin coated beads, more optionally, wherein the nanoparticles are streptavidin coated magnetic beads; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the sequencing primer site sequence comprises a Read 1 (R1) sequencing primer site sequence, more optionally, wherein the sequencing primer site sequence comprises a R1 sequencing primer site sequence of SEQ ID NO:5 or SEQ ID NO:6; and/or
optionally, wherein the sequencing primer site sequence comprises a Read 2 (R2) sequencing primer site sequence, more optionally, wherein the sequencing primer site sequence comprises a R2 sequencing primer site sequence of SEQ ID NO:7 or SEQ ID NO:8; and/or
optionally, wherein the sequencing primer site sequence comprises a Read 1 (R1) sequencing primer site sequence, more optionally, wherein the sequencing primer site sequence comprises a R1 sequencing primer site sequence of SEQ ID NO:5 or SEQ ID NO:6; and/or
optionally, wherein the sequencing primer site sequence comprises a Read 2 (R2) sequencing primer site sequence, more optionally, wherein the sequencing primer site sequence comprises a R2 sequencing primer site sequence of SEQ ID NO:7 or SEQ ID NO:8; and/or
optionally, wherein the UMI sequence comprises 8 to 20 nucleotides, more optionally wherein the UMI sequence comprises 8 to 16 nucleotides; and/or
optionally, wherein the spatial address sequence comprises 8 to 50 nucleotides, yet more optionally wherein the spatial address sequence comprises 10 to 30 nucleotides; and/or
optionally, wherein the oligo d(T) sequence comprises 16 to 20 nucleotides; and/or
optionally, wherein the cell membranes are lysed by using a mild detergent,
   optionally, wherein the cell membranes are lysed using a mild detergent selected from NP40, Tween 20, Bile Salts, Triton X100, and (3-((3-cholamidopropyl)dimethylammonio)-1-propanesulfonate) (CHAPS); and/or
optionally, wherein the barcode nucleotide sequence comprises 5 to 20 nucleotides, more optionally, where the barcode nucleotide sequence comprises 10 to 15 nucleotides; and/or
optionally, wherein the dsDNA dye is a cyanine-based dye, more optionally, wherein the dsDNA dye is a cyanine-based dye selected from SYBR Green I, PicoGreen, SYBR Safe, SYBR Gold, thiazole orange, oxazole yellow, Safe-Green and Chai Green.

28. The spatial multi-omics method of aspect 27, wherein the dsDNA dye is Picogreen.

29. The spatial multi-omics method of aspect 27 or aspect 28, wherein the imaging and decoding the sets of beads is performed simultaneously.

30. The spatial multi-omics method of any one of aspects 27 to 29, wherein steps (E) and (E′) are performed sequentially or simultaneously.

31. A method for in situ decoding of spatially addressed nano-particles, comprising:
(A) infusing nanoparticles comprising immobilized oligos into tissue, the oligos comprising an adapter sequence, a sequence primer site sequence, a spatial address sequence, and an oligo d(T) sequence;
(B) capturing mRNA in situ by hybridizing mRNA in the tissue with the oligo d(T) sequence of the oligos;
(C) reverse transcribing the oligos comprising captured poly-A RNA transcripts in situ to form a nanoparticle bound cDNA construct;
(D) mapping the cDNA construct in situ by:
   (i) direct in situ sequencing of the spatial address sequence; or
   (ii) using decoding-by-hybridization approach to decode the spatial address sequence in situ; and
(E) digesting the tissue with a detergent and Proteinase K, and isolating the tagged constructs;
(F) separating the cDNA construct from the nanoparticle, library prepping the cDNA construct and sequencing the cDNA construct,
optionally, wherein the nanoparticles are streptavidin coated beads, more optionally, wherein the nanoparticles are streptavidin coated magnetic beads; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the sequencing primer site sequence comprises a Read 1 (R1) sequencing primer site sequence, more optionally, wherein the sequencing primer site sequence comprises a R1 sequencing primer site sequence of SEQ ID NO:5 or SEQ ID NO:6; and/or
optionally, wherein the sequencing primer site sequence comprises a Read 2 (R2) sequencing primer site sequence, more optionally, wherein the sequencing primer site sequence comprises a R2 sequencing primer site sequence of SEQ ID NO:7 or SEQ ID NO:8; and/or
optionally, wherein the spatial address sequence comprises 8 to 50 nucleotides, yet more optionally wherein the spatial address sequence comprises 10 to 30 nucleotides; and/or
optionally, wherein the oligo d(T) sequence comprises 16 to 20 nucleotides; and/or
optionally, wherein the detergent is Tween 20 and/or Triton X100.

32. A method for in situ decoding of spatially addressed nano-particles, comprising:
(A) infusing nanoparticles comprising multiple sets of immobilized oligos into a tissue, the multiple sets of immobilized oligos comprising:
a first set of oligos comprising an adapter sequence, a sequence primer site, a spatial address sequence, and an oligo d(T) sequence,
a second set of oligos comprising a spatial address sequence and a Sbs/ME sequence,
an optional third set of oligos comprising the adapter sequence, a spatial address sequence, and a Sbs/ME sequence,
wherein the first and third set of oligos are attached to the nanoparticles by a selectively cleavable linker, and wherein the second set of oligos are not attached to the nanoparticles by a selectively cleavable linker;
(B) capturing mRNA in situ by hybridizing mRNA in the tissue with the oligo d(T) sequence of the first set of oligos;
(C) reverse transcribing the oligos comprising captured poly-A RNA transcripts in situ to form a nanoparticle bound cDNA construct;
(D) extracting ex situ the cDNA construct from the nanoparticle by cleaving the cleavable linker and separating the cDNA construct from the nanoparticle;
(E) mapping the second set of oligos in situ by:
   (i) direct in situ sequencing of the spatial address sequence; or
   (ii) using decoding-by-hybridization approach to decode the spatial address sequence in situ; and
(F) library prepping the cDNA construct and sequencing the cDNA construct,
optionally, wherein the nanoparticles are streptavidin coated beads, more optionally, wherein the nanoparticles are streptavidin coated magnetic beads; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the sequencing primer site sequence comprises a Read 1 (R1) sequencing primer site sequence, more optionally, wherein the sequencing primer site sequence comprises a R1 sequencing primer site sequence of SEQ ID NO:5 or SEQ ID NO:6; and/or
optionally, wherein the sequencing primer site sequence comprises a Read 2 (R2) sequencing primer site sequence, more optionally, wherein the sequencing primer site sequence comprises a R2 sequencing primer site sequence of SEQ ID NO:7 or SEQ ID NO:8; and/or
optionally, wherein the spatial address sequence comprises 8 to 50 nucleotides, yet more optionally wherein the spatial address sequence comprises 10 to 30 nucleotides; and/or
optionally, wherein the oligo d(T) sequence comprises 16 to 20 nucleotides; and/or
optionally, the selectively cleavable linker is selected from biotin-based molecules, PC Linker, and a recognition site for a rare-cutter enzyme, more optionally, the selectively cleavable linker comprises desthiobiotin molecule(s) (ddBio); and/or
optionally, wherein the selectively cleavable linker can be cleaved by heating, competitive binding, pH change, chemical cleavage, enzymatic cleavage and/or photo-cleavage, more optionally, wherein the selectively cleavable linker can be cleaved by competitive binding and heat, more optionally, the selectively cleavable linker is cleaved by adding biotin and heating.

33. The method of aspect 31 or aspect 32, wherein the nanoparticles are functionalized with biological triggers for endocytosis and intracellular transport, or with targeting moieties or ligands for specific subcellular organelles.

34. The method of any one of aspects 31 to 33, wherein the nanoparticles further comprise lanthanide nanophosphor labels or Q-dot particles.

35. A spatial genomics method using ex situ spatial capture on a beadchip, comprising:
(A) loading beads into wells of a beadchip, wherein the beads comprise oligos that attached to the nanoparticles through a selectively cleavable linker, the oligos comprising a first adapter sequence, a spatial address sequence, and a first capture sequence;
(B) decoding the spatial address sequences of the oligos to determine the x,y position of the beads;
(C) placing tissue into sectioned areas of the beadchip, lysing the cell membranes, and adding non-tethered pool of oligos comprising a second adapter sequence, an optional UMI sequence, a sample index sequence, and a second capture sequence, wherein the first and second capture sequence bind to different portions of a biomolecule;
(D) capturing the biomolecule to the first and second capture sequence of the oligos;
(E) extending and/or ligating the first capture sequence to the second capture sequence to form a construct which comprises the first adapter sequence, the spatial address sequence, the first capture sequence, a gap or bridge molecule of interest, the second capture sequence, the optional UMI sequence, and the second adapter sequence; and
(F) obtaining ex situ spatial genomics information of the tissue by sequencing the construct using a sequencer and mapping the sequencing reads with x,y positions of the bead,
optionally, wherein the wells are nanowells; and/or
optionally, wherein the first adapter sequence comprises a P5 adapter sequence and the second adapter sequence comprises a P7 adapter sequence, or vice versa, more optionally, wherein the first adapter sequence comprises the sequence of SEQ ID NO:1 and the second adapter sequence comprises the sequence SEQ ID NO:2, or vice versa; and/or
optionally, wherein the UMI sequence comprises 8 to 20 nucleotides, more optionally wherein the UMI sequence comprises 8 to 16 nucleotides; and/or
optionally, wherein the spatial address sequence comprises 8 to 50 nucleotides, more optionally, wherein the spatial address sequence comprises 10 to 30 nucleotides; and/or
optionally, the selectively cleavable linker is selected from biotin-based molecules, PC Linker, and a recognition site for a rare-cutter enzyme, more optionally, the selectively cleavable linker comprises desthiobiotin molecule(s) (ddBio); and/or
optionally, wherein the selectively cleavable linker can be cleaved by heating, competitive binding, pH change, chemical cleavage, enzymatic cleavage and/or photo-cleavage, more optionally, wherein the selectively cleavable linker can be cleaved by competitive binding and heat, more optionally, the selectively cleavable linker is cleaved by adding biotin and heating; and/or
optionally, wherein the cell membranes are lysed by using a mild detergent, optionally, wherein the cell membranes are lysed using a mild detergent selected from NP40, Tween 20, Bile Salts, Triton X100, and (3-((3-cholamidopropyl)dimethylammonio)-1-propanesulfonate) (CHAPS).

36. The method of aspect 35, wherein the first capture sequence and the second capture sequence having complementary sequences to a targeted gene.

37. The method of aspect 35 or aspect 36, wherein the gap or bridge molecule of interest comprise nucleotides up to a thousand nucleotides in length.

38. A spatial genomics Assay for Transposase-Accessible Chromatin with high-throughput sequencing (ATAC-Seq) method, comprising
(A) providing a substrate which comprises oligos attached to the substrate through a selectively cleavable linker, the oligos comprising an adapter sequence, a spatial address sequence, and a transposome hybridization region, wherein if the substrate is an ordered substrate, then the spatial address sequence of the oligos is optional;
(B) decoding the spatial address sequences of the oligos to determine the x,y positions of the oligos on the substrate, wherein if the substrate is an ordered substrate, then step (B) is optional;
(C) placing tissue on top of the substrate, and lysing the cell membranes to access chromatin regions in the tissue;
(D) tagmentating the chromatin regions with a transposome complex to form tagmented fragments, and removing the transposome complex;
(E) permeabilizing the tissue to allow diffusion of the tagmented fragments to the substrate;
(F) capturing the tagmented fragments to the substrate by hybridizing the tagmented fragments to the transposome hybridization region of the oligos;
(G) processing the captured tagmented fragments to make substrate bound genomic library constructs for sequencing;
(H) releasing the genomic library constructs from the substrate by selectively cleaving the linker;
(I) obtaining spatial genomics information of the tissue by sequencing the genomic library constructs using a sequencer and mapping the sequencing reads with x,y positions of the oligos.
optionally, wherein at least a portion of the substrate comprises a coating with streptavidin; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the spatial address sequence comprises 8 to 50 nucleotides, yet more optionally wherein the spatial address sequence comprises 10 to 30 nucleotides; and/or
optionally, wherein the transposome hybridization region comprises a sequence that is complementary to tagmented ends of polynucleotides generated by a transposome complex; and/or
optionally, the selectively cleavable linker is selected from biotin-based molecules, PC Linker, and a recognition site for a rare-cutter enzyme, more optionally, the selectively cleavable linker comprises desthiobiotin molecule(s) (ddBio); and/or
optionally, wherein the selectively cleavable linker can be cleaved by heating, competitive binding, pH change, chemical cleavage, enzymatic cleavage and/or photo-cleavage, more optionally, wherein the selectively cleavable linker can be cleaved by competitive binding and heat, more optionally, the selectively cleavable linker is cleaved by adding biotin and heating.

39. The spatial genomics ATAC-Seq method of aspect 38, wherein the transposome complex is a TN5 transposome complex.

40. The spatial genomics ATAC-Seq method of aspect 38 or aspect 39, wherein the substrate is a plate, a multiwell plate, a slide, a flowcell, or nanoparticles, optionally, wherein at least a portion of the substrate comprises a streptavidin coating.

41. The spatial genomics ATAC-Seq method of any one of aspects 38 to 40, wherein the substrate comprises areas of immobilized oligos separated by interstitial areas lacking immobilized oligos.

42. The spatial genomics ATAC-Seq method of aspect 41, wherein the substrate comprises islands or clusters of immobilized oligos separated by interstitial areas lacking immobilized oligos, and wherein each island or cluster of immobilized oligos has a spatial address sequence that is different from the spatial address sequence of the other islands or clusters of immobilized oligos.

43. The spatial genomics ATAC-Seq method of any one of aspects 38 to 42, wherein the spatial address sequences are decoded by using a decoding-by-hybridization method.

44. The spatial genomics ATAC-Seq method of any one of aspects 38 to 41, wherein the substrate is an ordered substrate and the x,y positions of the oligos on the substrate are predetermined or can be readily determined without having to decode the spatial address sequences in step (B).

45. The spatial genomics ATAC-Seq method of any one of aspects 38 to 44, wherein the cell membranes are lysed by using a mild detergent, optionally, wherein the mild detergent is selected from NP40, Tween 20, Bile Salts, Triton X100, and (3-((3-cholamidopropyl)dimethylammonio)-1-propanesulfonate) (CHAPS).

46. The spatial genomics ATAC-Seq method of any one of aspects 38 to 45, wherein the tissue is permeabilized by using a detergent and Proteinase K, optionally wherein the detergent is Tween 20 and/or Triton X100.

47. The spatial genomics ATAC-Seq method of any one of aspects 38 to 46, where the captured tagmented fragments are processed by using top strand ligation, hybridization to Y-shaped adaptors, and gap-filling ligations steps,
optionally, wherein the top strand ligation utilizes a DNA ligase, and/or,
optionally, wherein the gap-filling ligation step comprises use of a DNA polymerase lacking strand-displacement activity, dNTPs, and a DNA ligase, more optionally, the DNA polymerase lacking strand-displacement activity is selected from a T4-based polymerase, a T7-based polymerase, a Pfu-based polymerase, or a Taq-based polymerase, and/or
optionally, wherein the Y-shaped adapters are noncomplementary at their ends to prevent their self-ligation and thus form a Y shape after annealing to the oligos.

48. The spatial genomics ATAC-Seq method of any one of aspects 38 to 46, wherein prior to sequencing the library constructs are amplified using PCR.

49. The spatial genomics ATAC-Seq method of any one of aspects 38 to 47, wherein the sequencer utilizes sequencing by synthesis technology.

50. The spatial genomics ATAC-Seq method of any one of aspects 38 to 48, wherein the hybridization region of the transposome complex is initially blocked by use of an accessory oligo that is then removed in step (E).

51. A spatial multi-omics Assay for Transposase-Accessible Chromatin with high-throughput sequencing (ATAC-Seq) and RNA-Seq method, comprising:
(A) providing a substrate which comprises multiple sets of immobilized oligos attached to the substrate through selectively cleavable linker(s), wherein the substrate comprises a first set of oligos which comprises a first adapter sequence, a spatial address sequence, and a transposome complex hybridization region, and a second set of oligos that comprises a second adapter sequence, a sequencing primer site sequence, a spatial address sequence, a unique molecular identifier (UMI) sequence, and an oligo(dT) sequence, wherein if the substrate is an ordered substrate, then the spatial address sequence of the first set of oligos, and/or second set of oligos are optional;
(B) decoding the spatial address sequences of the oligos to determine the x,y position of the oligos on the substrate, wherein if the substrate is an ordered substrate, then step (B) is optional;
(C) placing tissue on top of the substrate, and lysing the cell membranes to access poly-A RNA transcripts and chromatin regions in the tissue;
(D) tagmentating the chromatin regions with a first transposome complex to form tagmented fragments, and removing the first transposome complex;
(E) permeabilizing the tissue to allow diffusion of the tagmented fragments and poly-A RNA transcripts to the substrate;
(F) capturing the tagmented fragments and poly-A RNA transcripts to the substrate by (i) hybridizing the tagmented fragments to the transposome hybridization region of the first set of oligos, and (ii) hybridizing the poly-A RNA transcripts to the oligo(dT) sequence of the second set of oligos;
(G′) processing the first set of oligos comprising captured tagmented fragments to make substrate bound genomic library constructs for sequencing;
(G″) reverse transcribing the second set of oligos comprising captured poly-A RNA transcripts in the presence of a single stranded template switch oligo to make substrate bound cDNA library constructs for sequencing;
(H) releasing the genomic library constructs and the cDNA library constructs from the substrate by selectively cleaving the linker(s);
(I) amplifying the genomic library constructs and cDNA library constructs using PCR and splitting the amplified products into two portions comprising both amplified constructs;
(J) amplifying a first portion of the amplified constructs with a first adapter primer and a second adapter primer to form an ATAC-Seq library, wherein the first adapter primer and the second adapter primer bind to first adapter sequence and second adapter sequence, respectively, and wherein the first adapter primer and/or the second adapter primer further comprises an index sequence and/or a sequencing primer site sequence;
(J′) tagmentating a second portion of the amplified constructs with a primer comprising the sequence primer sequence and a transposome complex (TSM) and then amplifying the tagmented amplified library constructs using PCR with the first adapter primer comprising a sequencing primer site sequence and the second adapter primer comprising an index sequence and a sequencing primer site sequence to form an RNA-Seq library, wherein the first adapter primer and the second adapter primer have difference sequence primer sequences; and
(K) obtaining multi-omics information by sequencing the ATAC-Seq library and RNA-Seq library using a sequencer and mapping the sequencing reads with x,y positions of the oligos,
optionally, wherein the first adapter sequence comprises a P5 adapter sequence and the second adapter sequence comprises a P7 adapter sequence, or vice versa, more optionally, wherein the first adapter sequence comprises the sequence of SEQ ID NO:1 and the second adapter sequence comprises the sequence SEQ ID NO:2, or vice versa; and/or
optionally, wherein the first adapter primer comprises a complementary P5 adapter sequence and the second adapter primer comprises a complementary P7 adapter sequence, or vice versa, more optionally, wherein the first adapter primer comprises a sequence complementary to SEQ ID NO:1 and the second adapter primer comprises a sequence complementary to SEQ ID NO:2, or vice versa; and/or
optionally, wherein the UMI sequence comprises 8 to 20 nucleotides, more optionally wherein the UMI sequence comprises 8 to 16 nucleotides; and/or
optionally, wherein the spatial address sequence comprises 8 to 50 nucleotides, yet more optionally wherein the spatial address sequence comprises 10 to 30 nucleotides; and/or
optionally, wherein the transposome hybridization region comprises a sequence that is complementary to tagmented ends of polynucleotides generated by a transposome complex; and/or
optionally, wherein the sequencing primer site sequence of the first adapter primer comprises a Read 1 (R1) sequencing primer site sequence; and/or
optionally, wherein the sequencing primer site sequence of the second adapter sequence comprises a Read 2 (R2) sequencing primer site sequence; and/or
optionally, wherein the index sequence comprises 6 to 10 nucleotides; and/or
optionally, the selectively cleavable linker is selected from biotin-based molecules, PC Linker, and a recognition site for a rare-cutter enzyme, more optionally, the selectively cleavable linker comprises desthiobiotin molecule(s) (ddBio); and/or
optionally, wherein the selectively cleavable linker can be cleaved by heating, competitive binding, pH change, chemical cleavage, enzymatic cleavage and/or photo-cleavage, more optionally, wherein the selectively cleavable linker can be cleaved by competitive binding and heat, more optionally, the selectively cleavable linker is cleaved by adding biotin and heating.

52. The spatial multi-omics ATAC-Seq and RNA-Seq method of aspect 51, wherein the transposome complex is a TN5 transposome complex.

53. The spatial multi-omics ATAC-Seq and RNA-Seq method of aspect 51 or aspect 52, wherein the substrate is a plate, a multiwell plate, a slide, a flowcell, or nanoparticles, optionally, wherein at least a portion of the substrate comprises a streptavidin coating.

54. The spatial genomics ATAC-Seq method of any one of aspects 51 to 53, wherein the substrate comprises areas of immobilized oligos separated by interstitial areas lacking immobilized oligos.

55. The spatial multi-omics ATAC-Seq and RNA-Seq method of aspect 54, wherein the substrate comprises islands or clusters of the two sets of immobilized oligos separated by interstitial areas lacking immobilized oligos, and wherein each island or cluster of the immobilized oligos has a spatial address sequence that is different from the spatial address sequence of the other islands or clusters of immobilized oligos.

56. The spatial multi-omics ATAC-Seq and RNA-Seq method of any one of aspects 51 to 55, wherein the spatial address sequences are decoded by using a decoding-by-hybridization method.

57. The spatial multi-omics ATAC-Seq and RNA-Seq method of aspect 51 or aspect 52, wherein the substrate is an ordered substrate and the x,y positions of the oligos on the substrate are predetermined or can be readily determined without having to decode the spatial address sequences in step (B).

58. The spatial multi-omics ATAC-Seq and RNA-Seq method of any one of aspects 51 to 57, wherein the cell membranes are lysed by using a mild detergent, optionally, wherein the cell membranes are lysed using a mild detergent selected from NP40, Tween 20, Bile Salts, Triton X100, and (3-((3-cholamidopropyl)dimethylammonio)-1-propanesulfonate) (CHAPS).

59. The spatial multi-omics ATAC-Seq and RNA-Seq method of any one of aspects 51 to 58, wherein the tissue is permeabilized by using a detergent and Proteinase K, optionally wherein the detergent is Tween 20 and/or Triton X100.

60. The spatial multi-omics ATAC-Seq and RNA-Seq method of any one of aspects 51 to 59, where the captured tagmented fragments are processed by using top strand ligation, hybridization to Y-shaped adaptors, and gap-filling ligations steps.
optionally, wherein the top strand ligation utilizes a DNA ligase, and/or,
   optionally, wherein the gap-filling ligation step comprises use of a DNA polymerase lacking strand-displacement activity, dNTPs, and a DNA ligase, more optionally, the DNA polymerase lacking strand-displacement activity is selected from a T4-based polymerase, a T7-based polymerase, a Pfu-based polymerase, or a Taq-based polymerase, and/or
   optionally, wherein the Y-shaped adapters e are noncomplementary at their ends to prevent their self-ligation and thus form a Y shape after annealing to the oligos.

61. The spatial multi-omics ATAC-Seq and RNA-Seq method of any one of aspects 51 to 60, wherein prior to sequencing the library constructs are amplified using PCR.

62. The spatial multi-omics ATAC-Seq and RNA-Seq method of any one of aspects 51 to 61, wherein the sequencer utilizes sequencing by synthesis technology.

63. The spatial multi-omics ATAC-Seq and RNA-Seq method of any one of aspects 51 to 62, wherein the hybridization region of the transposome complex is initially blocked by use of an accessory oligo that is then removed in step (E).

64. The spatial multi-omics ATAC-Seq and RNA-Seq method of any one of aspects 51 to 63, wherein steps (G) and (G′) are performed sequentially or concurrently.

65. The spatial multi-omics ATAC-Seq and RNA-Seq method of any one of aspects 51 to 64, wherein steps (J) and (J′) are performed sequentially or concurrently.

66. The spatial multi-omics ATAC-Seq and RNA-Seq method of any one of aspect 51 to 65, wherein the oligo d(T) sequence is 16 to 20 nucleotides.

67. A spatial transcriptomics method, comprising:
(A) providing a substrate comprising features, wherein oligos are immobilized on the features of the substrate, wherein the oligos comprise an adapter sequence, a spatial address sequence, an optional sequence primer site, and an oligo(dT) sequence, wherein if the substrate is an ordered substrate, then the spatial address sequence of the oligos is optional;
(B) decoding the spatial address sequences of the oligos to determine their x,y position in the substrate, wherein if the substrate is an ordered substrate, then step (B) is optional;
(C) placing tissue onto the substrate, and lysing the cell membranes to access poly-A RNA transcripts in the tissue;
(D) capturing the poly-A RNA transcripts to the oligos;
(E) reverse transcribing the oligos comprising captured poly-A RNA transcripts: (i) in the presence of a single stranded template switch oligo to make substrate bound cDNA library constructs for sequencing, or (ii) further performing a single strand ligation reaction with a single stranded oligo to make substrate bound cDNA library constructs for sequencing;
(F) amplifying the oligo bound cDNA library constructs using PCR with an adapter primer;
(G) obtaining transcriptomics information by sequencing the amplified cDNA library constructs using a sequencer and mapping the sequencing reads with x,y positions of the oligos on the features of the substrate,
optionally, wherein at least a portion of the substrate comprises a coating of streptavidin; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the adapter primer comprises a complementary P5 adapter sequence or a complementary P7 adapter sequence, more optionally, wherein the first adapter primer comprises a sequence complementary to SEQ ID NO:1 or a sequence complementary to SEQ ID NO:2, and/or
optionally, wherein the spatial address sequence comprises 8 to 50 nucleotides, yet more optionally wherein the spatial address sequence comprises 10 to 30 nucleotides; and/or
optionally, the tissue is placed in removable incubation chamber(s) that is affixed to the top of the substrate; and/or
optionally, wherein the cell membranes are lysed by using a mild detergent, optionally, wherein the cell membranes are lysed using a mild detergent selected from NP40, Tween 20, Bile Salts, Triton X100, and (3-((3-cholamidopropyl)dimethylammonio)-1-propanesulfonate) (CHAPS).

68. The spatial transcriptomics method of aspect 67, wherein the substrate is a microarray, a plate, a multiwell plate, or a flowcell.

69. The spatial multi-omics ATAC-Seq and RNA-Seq method of aspect 67 or aspect 68, wherein the substrate is an ordered substrate and the x,y positions of the oligos on the features of the substrate are predetermined or can be readily determined without having to decode the spatial address sequences in step (B).

70. The spatial transcriptomics method of any one of aspects 67 to 69, wherein the substrate has a feature density (feature/mm²) of greater than 300000.

71. The spatial transcriptomics method of any one of aspects 67 to 69, wherein the features of the substrate and/or oligos further comprise lanthanide nanophosphor labels.

72. A spatial multi-omics method to detect targeted RNA and proteins, comprising:
(A) providing a substrate comprising features having lanthanide nanophosphor labels, wherein the features comprise multiple sets of immobilized oligos, wherein a first set of oligos comprise an adapter sequence, a spatial address sequence, an optional sequence primer site, and an oligo(dT) sequence, and a second set oligos that comprise a spatial address sequence, a UMI sequence, and a capture sequence, and wherein if the substrate is an ordered substrate, then the spatial address sequence of the first set of oligos and/or the second set of oligos are optional;
(B) decoding the spatial address sequences of the oligos to determine their x,y position in the substrate, wherein if the substrate is an ordered substrate, then step (B) is optional;
(C) placing tissue on top of the substrate, and lysing the cell membranes to access poly-A RNA transcripts and proteins in the tissue;
(D) adding antibodies comprising a barcode nucleotide sequence which bind with specificity to a targeted protein(s);
(E) capturing the poly-A RNA transcripts to the first set of oligos;
(E′) capturing the antibodies to the second set of oligos by hybridizing the barcode nucleotide sequence of the antibodies to the capture sequence;
(F) reverse transcribing the oligos comprising captured poly-A RNA transcripts with oNTPS;
(G) adding a primer which binds to oligos on the features of the substrate and extending the primers with oNTPS;
(H) adding a dsDNA dye that binds to the extended primer sequences; and
(I) detecting targeted RNA and proteins by imaging, and determining the x,y positions of the first and second sets of oligos on the features of the substrate,
optionally, wherein at least a portion of the substrate comprises a coating of streptavidin; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the UMI sequence comprises 8 to 20 nucleotides, more optionally wherein the UMI sequence comprises 8 to 16 nucleotides; and/or
optionally, wherein the spatial address sequence comprises 8 to 50 nucleotides, yet more optionally wherein the spatial address sequence comprises 10 to 30 nucleotides; and/or
optionally, wherein the oligo d(T) sequence comprises 16 to 20 nucleotides; and/or
optionally, wherein the cell membranes are lysed by using a mild detergent,
   optionally, wherein the cell membranes are lysed using a mild detergent selected from NP40, Tween 20, Bile Salts, Triton X100, and (3-((3-cholamidopropyl)dimethylammonio)-1-propanesulfonate) (CHAPS); and/or
optionally, wherein the barcode nucleotide sequence comprises 5 to 20 nucleotides, more optionally, where the barcode nucleotide sequence comprises 10 to 15 nucleotides.

73. The spatial multi-omics method of aspect 72, wherein the substrate is a microarray, a plate, a multiwell plate, or a flowcell.

74. The spatial multi-omics method of aspect 72 or aspect 73, wherein the substrate is an ordered substrate and the x,y positions of the oligos on the features of the substrate are predetermined, or can be readily determined without having to decode the spatial address sequences in step (B).

75. The spatial multi-omics method of any one of aspects 72 to 74, wherein the substrate has a feature density (feature/mm²) of greater than 300000.

76. The spatial multi-omics method of any one of aspects 72 to 75, wherein the dsDNA dye is a cyanine-based dye.

77. The spatial multi-omics method of aspect 76, wherein the cyanine-based dye is selected from SYBR Green I, PicoGreen, SYBR Safe, SYBR Gold, thiazole orange, oxazole yellow, Safe-Green and Chai Green, optionally, wherein the dsDNA dye is PicoGreen.

78. The spatial multi-omics method of any one of aspects 72 to 77, wherein steps (E) and (E′) are performed sequentially or concurrently.

79. A method for in situ decoding of spatially addressed oligos, comprising:
(A) infusing an oligo-coated substrate into tissue, the oligo-coated substrate comprising immobilized oligos comprising an adapter sequence, a sequence primer site, a spatial address sequence, and an oligo d(T) sequence;
(B) capturing mRNA in situ by hybridizing mRNA in the tissue with the oligo d(T) sequence of the oligos;
(C) reverse transcribing the oligos that comprise captured poly-A RNA transcripts in situ to form a substrate bound cDNA construct;
(D) mapping the cDNA construct in situ by:
   (i) direct in situ sequencing of the spatial address sequence; or
   (ii) using decoding-by-hybridization approach to decode the spatial address sequence in situ; and
(E) digesting the tissue with a detergent and Proteinase K, and isolating the substrate bound cDNA construct; and
(F) separating the cDNA construct from the substrate, library prepping the cDNA construct and sequencing the cDNA construct.
optionally, wherein at least a portion of the substrate comprises streptavidin; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the spatial address sequence comprises 8 to 50 nucleotides, yet more optionally wherein the spatial address sequence comprises 10 to 30 nucleotides; and/or
optionally, wherein the oligo d(T) sequence comprises 16 to 20 nucleotides; and/or
optionally, wherein the detergent is Tween 20 and/or Triton X100.

80. The method of aspect 79, wherein the substrate is from 10 nm to 10 μm in size.

81. The method of aspect 79 or aspect 80, wherein the substrate is functionalized with biological triggers for endocytosis and intracellular transport, or with targeting moieties or ligands for specific subcellular organelles.

82. The method of any one of aspects 79 to 82, wherein the substrate or oligos comprise lanthanide nanophosphor labels or Q-dot particles.

83. A method for in situ decoding of spatially addressed oligos, comprising:
(A) infusing an oligo-coated substrate into tissue, the oligo-coated substrate comprising multiple sets of immobilized oligos sets, the multiple sets of immobilized oligos comprising:
   a first set of oligos comprising a first adapter sequence, a sequence primer site, a spatial address sequence, and an oligo d(T) sequence,
   a second set of oligos comprising a spatial address sequence and a Sbs/ME sequence,
   an optional third set of oligos comprising the first adapter sequence, a spatial address sequence, and a Sbs/ME sequence,
wherein the first and third set of oligos are attached to the substrate by a selectively cleavable linker, and wherein the second set of oligos are not attached to the substrate by a selectively cleavable linker;
(B) capturing mRNA in situ by hybridizing mRNA in the tissue with the oligo d(T) sequence of the first set of oligos;
(C) reverse transcribing the oligos comprising captured poly-A RNA transcripts in situ to form a substrate bound cDNA construct;
(D) extracting ex situ the cDNA construct from the substrate by selectively cleaving the cleavable linker and separating the cDNA construct from the substrate;
(E) mapping the second set of oligos in situ by:
   (i) direct in situ sequencing of the spatial address sequence; or
   (ii) using decoding-by-hybridization approach to decode the spatial address sequence in situ; and
(F) library prepping the cDNA construct and sequencing the cDNA construct,
optionally, wherein at least a portion of the substrate comprises streptavidin; and/or
optionally, wherein the adapter sequence comprises a P5 adapter sequence or comprises a P7 adapter sequence, more optionally, wherein the adapter sequence comprises a sequence 98% identical to the sequence of SEQ ID NO:1 to SEQ ID NO:4, yet more optionally, wherein the adapter sequence comprises the sequence of SEQ ID NO:1 or SEQ ID NO:2; and/or
optionally, wherein the spatial address sequence comprises 8 to 50 nucleotides, yet more optionally wherein the spatial address sequence comprises 10 to 30 nucleotides; and/or
optionally, wherein the oligo d(T) sequence comprises 16 to 20 nucleotides; and/or
optionally, the selectively cleavable linker is selected from biotin-based molecules, PC Linker, and a recognition site for a rare-cutter enzyme, more optionally, the selectively cleavable linker comprises desthiobiotin molecule(s) (ddBio); and/or
optionally, wherein the selectively cleavable linker can be cleaved by heating, competitive binding, pH change, chemical cleavage, enzymatic cleavage and/or photo-cleavage, more optionally, wherein the selectively cleavable linker can be cleaved by competitive binding and heat, more optionally, the selectively cleavable linker is cleaved by adding biotin and heating.

84. The method of aspect 83, wherein the substrate is from 10 nm to 10 μm in size.

85. The method of aspect 83 or aspect 85, wherein the substrate is functionalized with biological triggers for endocytosis and intracellular transport, or with targeting moieties or ligands for specific subcellular organelles.

86. The method of any one of aspects 83 to 85, wherein the substrate or oligos comprise lanthanide nanophosphor labels or Q-dot particles.

87. A spatial genomics method using ex situ spatial capture on a substrate, comprising:
(A) providing a substrate comprising features, wherein the features comprise immobilized oligos that attached to the features of the substrate through a selectively cleavable linker, the oligos comprising a first adapter sequence, a spatial address sequence, and a first capture sequence, wherein if the substrate is an ordered substrate, then the spatial address sequence of the oligos is optional;
(B) decoding the spatial address sequences of the oligos to determine the x,y position of the beads, wherein if the substrate is an ordered substrate, then step (B) is optional;
(C) placing tissue on the substrate, lysing the cell membranes, and adding non-tethered pool of oligos comprising a second adapter sequence, an optional UMI sequence, a sample index sequence, and a second capture sequence, wherein the first and second capture sequence bind to different portions of a biomolecule;
(D) capturing the biomolecule to the first and second capture sequence of the oligos;
(E) extending and/or ligating the first capture sequence to the second capture sequence to form a construct which comprises the first adapter sequence, the spatial address sequence, the first capture sequence, a gap or bridge molecule of interest, the second capture sequence, the optional UMI sequence, and the second adapter sequence; and
(F) obtaining ex situ spatial genomics information of the tissue by sequencing the construct using a sequencer and mapping the sequencing reads with the determined x,y positions of the oligos,
optionally, wherein the wells of the substrate comprise a streptavidin coating; and/or
optionally, wherein the first adapter sequence comprises a P5 adapter sequence and the second adapter sequence comprises a P7 adapter sequence, or vice versa, more optionally, wherein the first adapter sequence comprises the sequence of SEQ ID NO:1 and the second adapter sequence comprises the sequence SEQ ID NO:2, or vice versa; and/or
optionally, wherein the spatial address sequence comprises 8 to 50 nucleotides, yet more optionally wherein the spatial address sequence comprises 10 to 30 nucleotides; and/or
optionally, the selectively cleavable linker is selected from biotin-based molecules, PC Linker, and a recognition site for a rare-cutter enzyme, more optionally, the selectively cleavable linker comprises desthiobiotin moieties(s) (ddBio); and/or
optionally, wherein the selectively cleavable linker can be cleaved by heating, competitive binding, pH change, chemical cleavage, enzymatic cleavage and/or photo-cleavage, more optionally, wherein the selectively cleavable linker can be cleaved by competitive binding and heat, more optionally, the selectively cleavable linker is cleaved by adding biotin and heating; and/or
optionally, wherein the cell membranes are lysed by using a mild detergent,
   optionally, wherein the cell membranes are lysed using a mild detergent selected from NP40, Tween 20, Bile Salts, Triton X100, and (3-((3-cholamidopropyl)dimethylammonio)-1-propanesulfonate) (CHAPS).

88. The spatial multi-omics method of aspect 87, wherein the substrate is a microarray, a plate, a multiwell plate, or a flowcell.

89. The spatial multi-omics method of aspect 87 or aspect 88, wherein the substrate is an ordered substrate and the x,y positions of the oligos on the features of the substrate are predetermined, or can be readily determined without having to decode the spatial address sequences in step (B).

90. The spatial multi-omics method of any one of aspects 87 to 89, wherein the substrate has a feature density (feature/mm²) of greater than 300000.

91. The method of any one of aspects 87 to 90, wherein the first capture sequence and the second capture sequence having complementary sequences to a targeted gene.

92. The method of any one of aspects 87 to 91, wherein the gap or bridge molecule of interest comprise nucleotides up to a thousand nucleotides in length.

The following examples are intended to illustrate but not limit the disclosure. While they are typical of those that might be used, other procedures known to those skilled in the art may alternatively be used.

### Examples

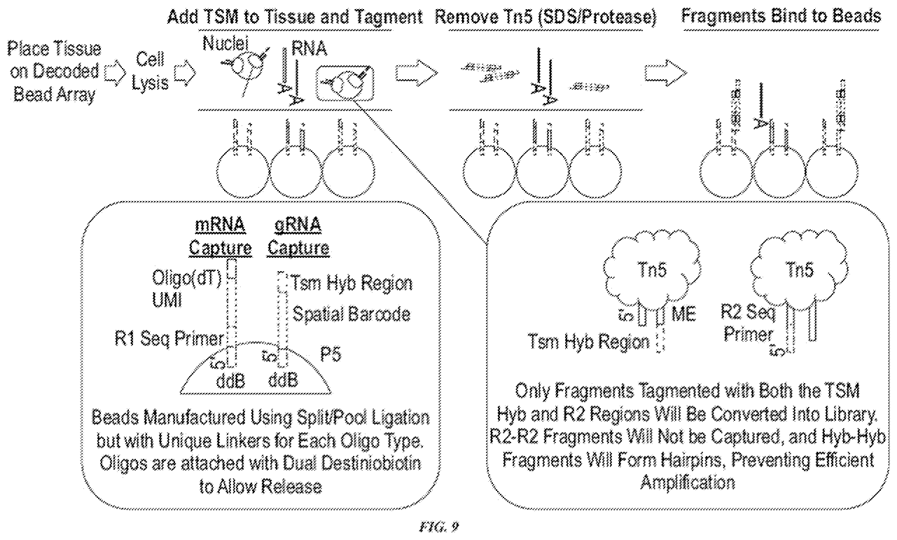

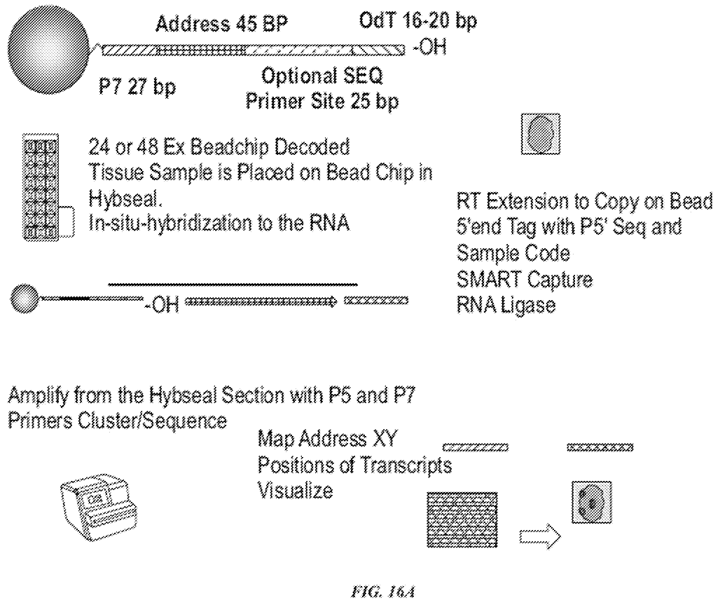

Performing spatial genomic assays using a bead-based array. A bead-based array can be used to extend spatial omic techniques to ATAC-Seq (e.g., see FIG. 8). The bead-based array uses beads that are coated with ddBio-labeled oligos containing a P5 sequence, a spatial barcode, and a transposome hybridization region. The transposons used in the spatial genomics assay contain the ME sequence along with an overhang termed the Tsm hyb region. Prior to starting the spatial genomics assay, the beads are loaded into a patterned substrate and the spatial barcodes decoded. This decoding method can include, but not limited to, a decoding-by-hybridization and by decoding-by-sequencing. In a particular embodiment, the decoding method is the decoding-by-hybridization method taught in Gunderson et al. (Genome Res. 14(5):870-874) (2004)) (e.g., see FIG. 15).

Next the tissue section is placed on top of the bead array, and cell membranes are lysed using a mild detergent. The open chromatin regions are tagmented with Tn5 transposomes, then the Tn5 is removed and the tissue further permeabilized with detergents and protease treatment. During this process, the tagmented fragments can diffuse down to the beads and be captured by the oligos through hybridization of the Tsm hyb region. Top strand ligation, hyb-to-Y, and gap-fill ligation steps of the spatial genomics assay can be performed on the slide. Oligos are then released with heat+addition of biotin to outcompete the ddBio, and PCR can be performed in tube. Final libraries are quantified and then loaded onto a sequencer to generate spatial genomic information.

One possible concern is that the Tn5 transposomes may hybridize to the bead array prior to tagmenting the nuclei. One way to address this is to initially block the hybridization region on the transposome with an accessory oligo. After tagmentation, excess transposome can be washed away and then the accessory oligo can be melted off during the SDS/protease treatment step. Alternatively, the tissue can be mounted on a separate surface (e.g., a cover slip) and then transferred to the bead array after tagmentation and washing of the tissue.

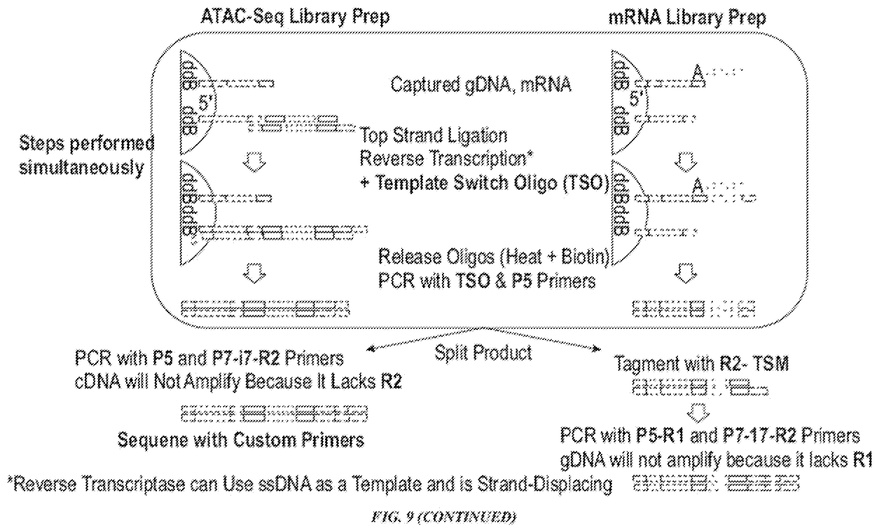

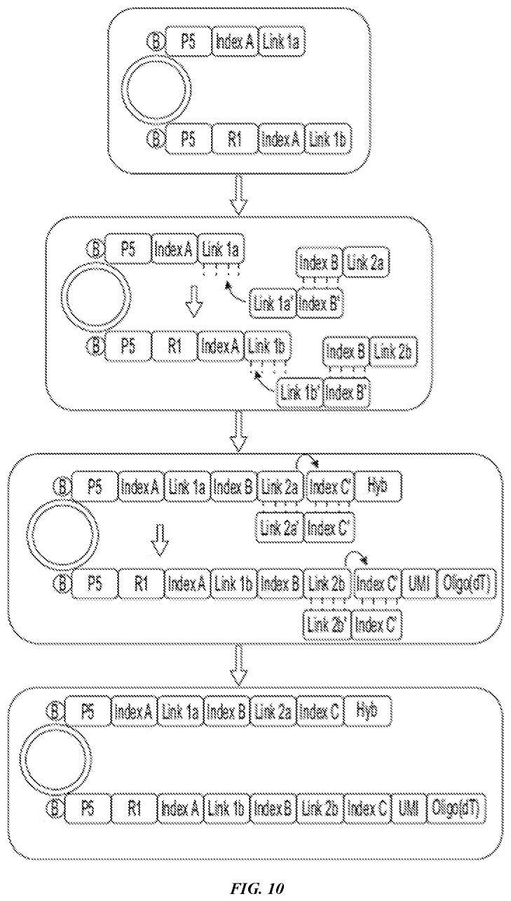

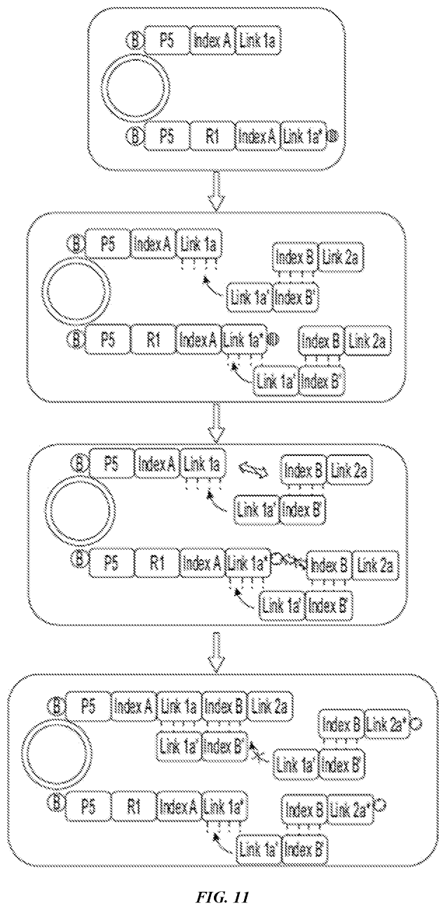

Performing spatial multi-omic assays using a bead-based array. The above ATAC-Seq workflow can be modified to be a spatial multi-omic assay. For example, the multi-omic assay can provide the information from both ATAC-Seq/RNA-Seq, with the later library prep steps modeled around the SNARE-Seq approach (e.g., see FIG. 9). For this assay, the beads are slightly modified from above, such that there are two types of oligos on each bead, both with the same spatial barcode. The gDNA capture oligos are the same as described above, but in addition there are also mRNA capture oligos with P5 and the R1 sequencing primer, followed by the spatial barcode, a UMI, and an oligo(dT) sequence. Assembly of this type of bead is possible by modification of a split-pool technique used for bead assembly. In one method, using different linker regions for the two oligos types confers specificity of assembly (e.g., see FIG. 10). In a second method, incorporation of blocking/deblocking steps allows the use of the same linker sequences, reducing the number of unique oligos that must be synthesized (e.g., see FIG. 11). This method may also confer improved specificity of oligo assembly. The spatial multi-omic assay uses two types of transposomes, one with the Tsm hyb region and one with the R2 sequencing primer. To begin, the tissue section is placed the bead array, which has been decoded as above, followed by a mild cell lysis (e.g., see FIG. 9). Tn5 transposome is added to the tissue, followed by tagmentation, Tn5 removal and tissue permeabilization, and capture of the fragments by the bead array. Because oligo(dT)-tailed oligos are also included on the beads, poly-A RNA transcripts will also be captured.

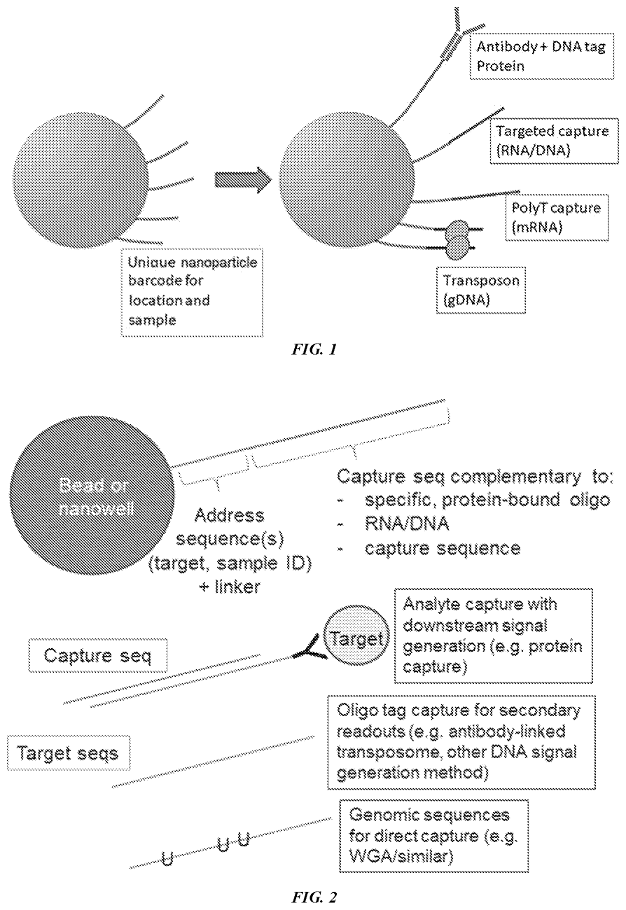

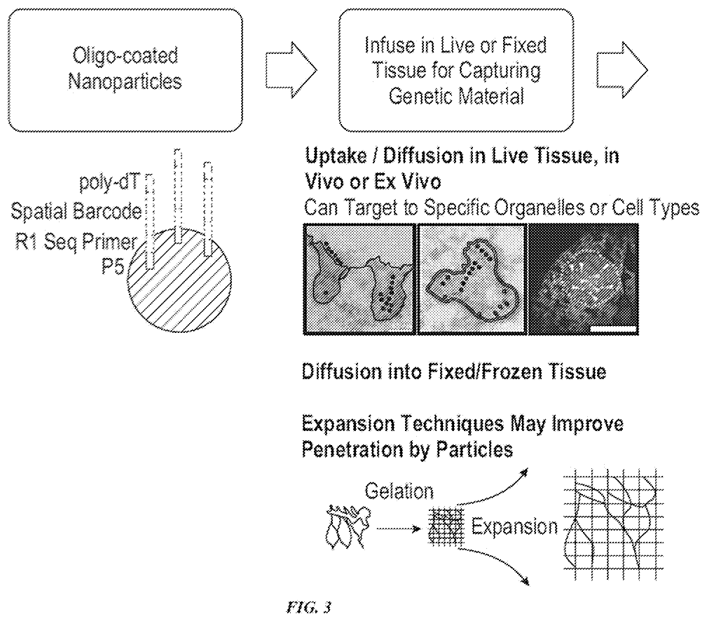

The spatial multi-omic assay simultaneously provides steps for ATAC-Seq and RNA-Seq libraries for sequencing. Top strand ligation with DNA ligase links the spatial barcode to the gDNA fragment for the ATAC-Seq libraries, but has no effect on the RNA. Next reverse transcription copies the RNA fragment onto the spatially barcoded oligo. This step also fills in the gaps for the ATAC-Seq libraries, as reverse transcriptase can use ssDNA as a template and has strand displacing activity. Reverse transcriptase also exhibits template switch activity, so inclusion of a template switch oligo allows the addition of a PCR primer landing site to the 3′ ends of the fragments. After this step, the oligos are released using heat and biotin, and PCR can be performed to amplify both ATAC-Seq and RNA-Seq fragments in the same tube. After PCR amplification, the product is split. One half undergoes PCR with P5 and P7-i7-R2 primers to preferentially amplify the ATAC-Seq library and add i7 indices. The other half undergoes tagmentation with R2-TSM followed by PCR with P5-R1 and P7-i7-R2 primers to preferentially amplify the RNA-Seq libraries. Then the samples are quantified and loaded onto the sequencer, with the ATAC-Seq libraries sequenced using primers. Partitioning the two library types prior to loading on the sequencer ensures that coverage and read lengths are appropriate for each type of library. This type of approach can be extended to assay more analytes (e.g., proteins using DNA-tagged antibodies) and to assay specific targets by incorporating the appropriate capture sequences onto the beads (e.g., see FIG. 1).

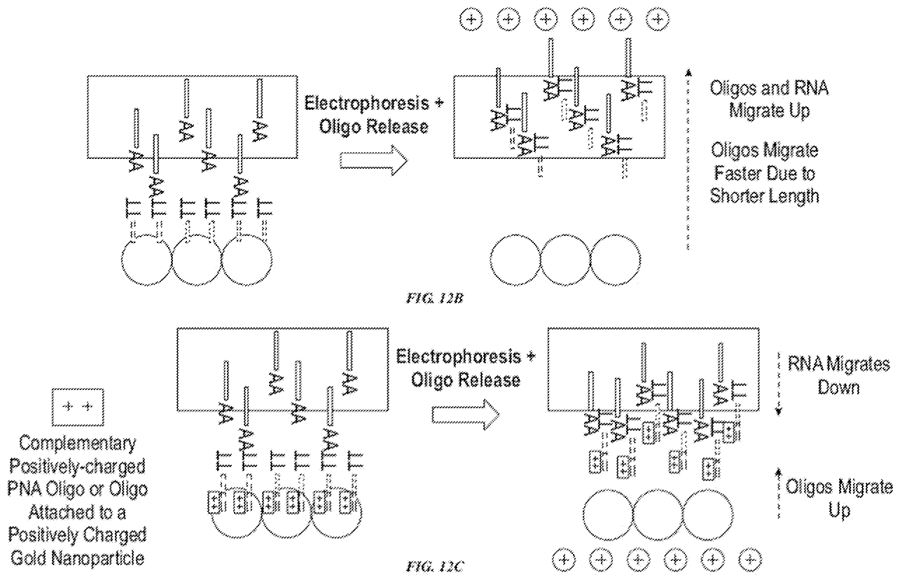

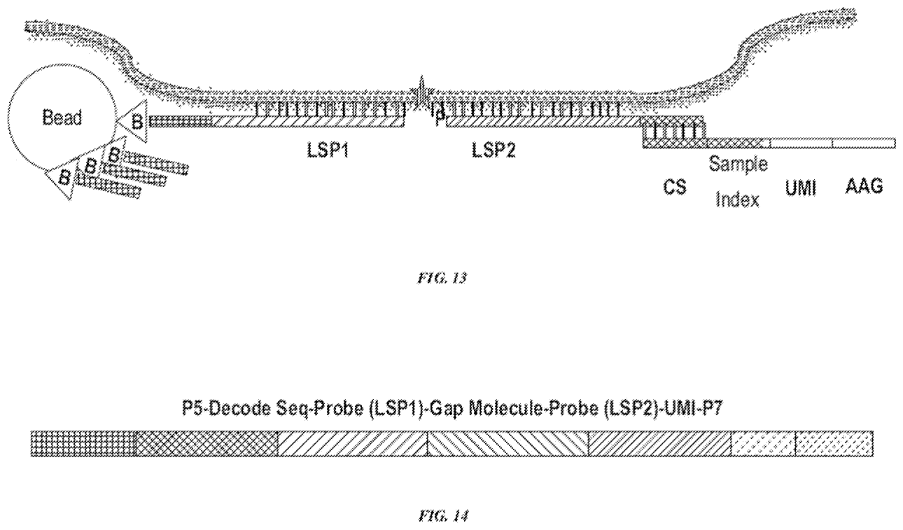

Spatial omics assay that utilizes a bead based genotyping strategy for ex situ spatial capture. The idea is to use an identifying code of the x,y space attached to the capture a probe (e.g., LSP1) in the library prep assay (e.g., see FIG. 13). These beads are then dropped into an array and the x,y positions are decoded using decoding-by-hybridization (e.g., see FIG. 15) or by decoding-by-sequencing. The sample section is placed on the array and the nucleic acids and or proteins are eluted to the surface of the bead array, the material is captured through the library prep process and capture of the sequence of interest using a second probe (e.g., LSP2). The library is recovered from the slide and the location and SNV (or other bridging molecule) of interest is sequenced and the XY distribution of the signal is mapped and displayed.

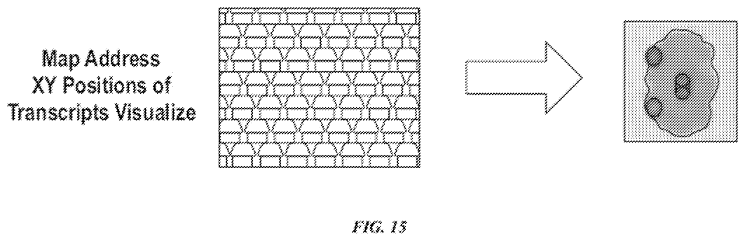

Beads are generated which comprises a p5 sequence, an identifying code of the x,y space (i.e., address sequence) and a first capture sequence (e.g., LSP1) that are tethered to the bead with biotin containing residue(s). The identifying code is typically from 10 to 60 bp. The first capture sequence is typically from 25 to 45 bp. The oligonucleotides may be attached to the beads by sequence and arrayed, or the pools of beads may be made using standard bead pool chemistry. The beads are placed in an array and the x,y positions are decoded using. Another pool of oligonucleotides is created that are not tethered to a substrate, and which comprise a P7 sequence, a second capture sequence (LSP2), an index sequence, and optionally an UMI tag. A tissue sample is sectioned and placed on top of the beads and treated to release target molecules (e.g., DNA, RNA, polypeptides) which are then tethered to the beads by hybridizing to the capture sequence. Alternatively, the tissue can be stained with a nucleic acid tagged with a mAb prior to being placed on top of the beads. The nontethered pool of the oligonucleotides are then added and hybridize to complementary sequences in target molecules. After washing to remove unbound oligonucleotides and non-targeted molecules, an extension and ligation step is performed to form a construct (e.g., see FIG. 14). The construct may be captured on the bead or in the sample segmented well on the slide of the library. The library constructs are then clustered and sequenced. The results are then mapped and the spatial data is determined.

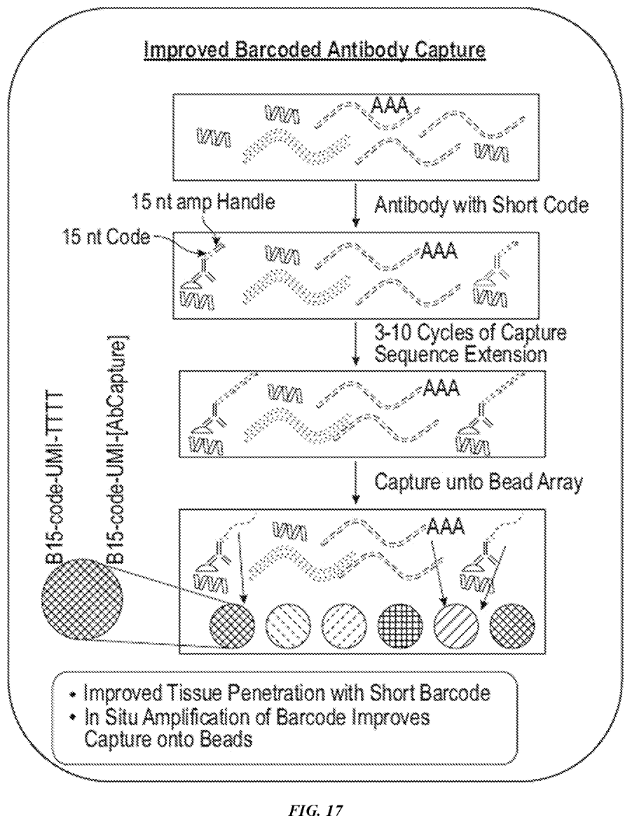

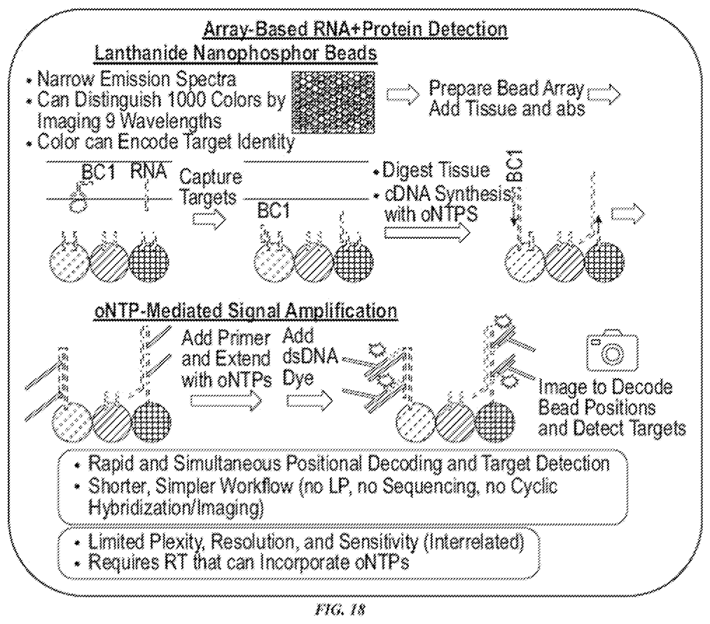

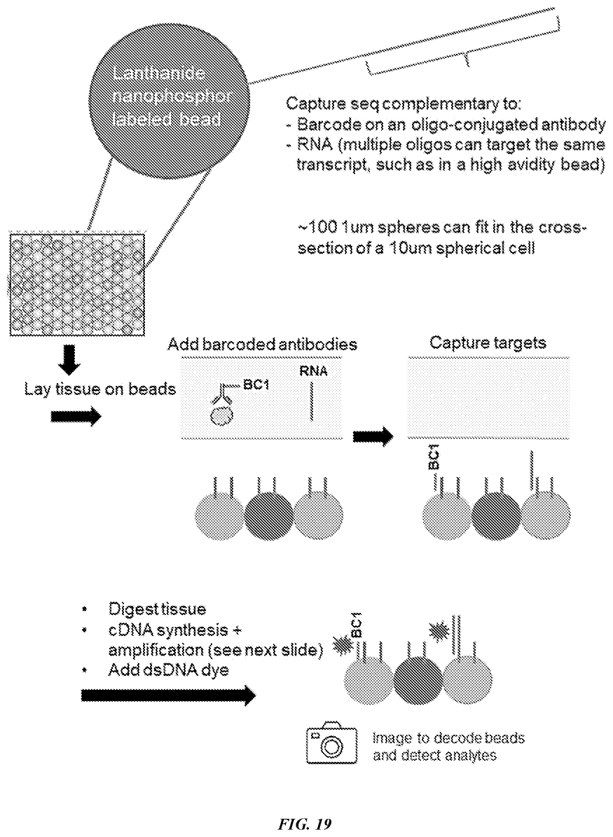

Spatial multiomics assay that leverages combined RNA/protein detection using a bead or nanoparticle-based array. FIG. 17 provides a method that provides for in situ amplification and capture of mRNA and antibodies in a bead-based array. As shown, the protein analytes of interest are bound by antibodies in situ. The antibodies comprise and appended nucleic acid sequence that can be extended. After 3-10 cycles of capture sequence extension, the antibody/protein analytes are capture on beads. The beads comprise at least 2 types of probes, one probe can capture the antibody/protein analytes via the appended nucleic acid sequence, while the other probe comprising a capture moiety of oligo(dT) can hybridize directly to mRNA. The probes have an address sequence and therefore their x,y position can be identified on an array, e.g., beadchip. FIGS. 18 and 19 provides further embodiments of the foregoing method by providing that the beads can be lanthanide nanophosphor beads, which have a narrow emission spectra and targets can identified based upon color. The lanthanide nanophosphor beads are ˜1 μm in diameter, and ˜100 lanthanide nanophosphor beads can fit into a nanowell having ˜10 μm in diameter. After capture of the antibody/analytes and RNA on to the beads, the tissue is digested away, typically by use of proteinase K and SDS, and cDNA synthesis is performed with oNTPs. The oNTP-mediated signal is then amplified, the address sequences are decoded and the bound targeted analytes are imaged by using a dsDNA dye. Some considerations for the foregoing method includes the following: Spatial resolution, sensitivity, and target plexity are interrelated (More targets→fewer beads per target adjacent to a cell→reduced resolution/sensitivity); if sensitivity, plexity are more important than spatial resolution, lateral diffusion could be increased (e.g., by heat) to allow analytes to encounter more beads; amplification may be needed to improve sensitivity; and simultaneous decoding/detection possible because dsDNA dyes like Picogreen excite at 485 nm while nanophosphors excite at 280 nm/IR. Some advantages for the foregoing methods include: rapid and simultaneous positional decoding and target detection; reduced COGS and shorter, simpler workflow (no LP, no sequencing, no cyclic hybridization/imaging); and the method leverages ILMN bead/array expertise.

Spatial genomics using in situ decoding of barcoded nanoparticles. A method for spatially mapping single-cell transcriptomes involves the use of a surface coated with barcoded oligonucleotides, where the spatial location of each barcode is known. One implementation of this approach is with a bead array and the RNA molecules within the tissue diffuse to the beads and are captured by oligo(dT)-containing barcoded oligonucleotides. The captured RNA is reverse transcribed into cDNA, linking the address barcode with the cDNA sequence. This is followed by library prep and sequencing using next generation sequencing platforms. During analysis, the spatial barcode is used to map the physical location of the read is derived from. Proteins can also be mapped with this approach using oligo-conjugated antibodies or aptamers. Disadvantages of the foregoing method include: low sensitivity (i.e., many mRNA molecules are not detected); loss of spatial information in the vertical direction; incompatibility with tissue blocks, tissues must be sectioned to ˜10 μm and sections are mounted on a surface, which is laborious and time-consuming, enabling mapping of transcriptomes in tissue blocks would be especially advantageous for neuroscience applications, as neuronal processes can span multiple sections and exhibit mRNA translation; and inability to target specific subcellar organelles.

In order to overcome the foregoing issues, the disclosure provides a method for spatial omic platforms and systems that utilize barcoded oligonucleotides that are decoded in situ. For the in situ method, tissues will be infused with nanoparticles coated with barcoded oligos that can bind mRNA (and/or protein or DNA). 3D in situ imaging of the nanoparticles can be used to decode the barcode. mRNA can be copied onto the barcoded oligos through reverse transcription; the cDNA molecules are then extracted from the tissue, followed by library prep and sequencing to generate spatially localized transcriptomes. By infusing tissue with the barcoded nanoparticles would overcome the following issues: low sensitivity, by infusing nanoparticles throughout the tissue may limit the distance that mRNA would have to travel before being bound by the barcoded oligos, thereby improving sensitivity; loss of spatial information in the vertical direction, in situ imaging of the beads can be performed in multiple optical planes; incompatibility with tissue blocks, nanoparticles can be infused into blocks of tissue or tissue sections, and imaging can be performed in 3 dimensions; inability to target specific subcellular organelles, nanoparticles can be functionalized to target specific subcellular organelles.

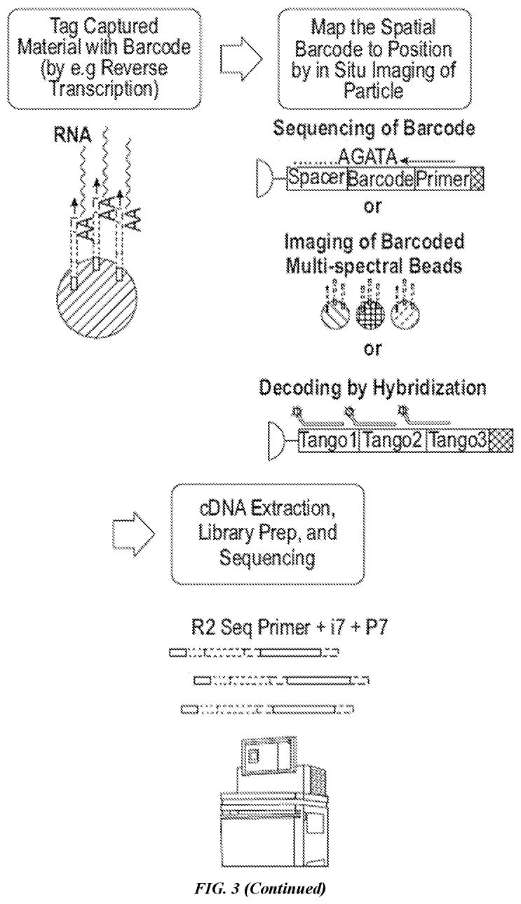

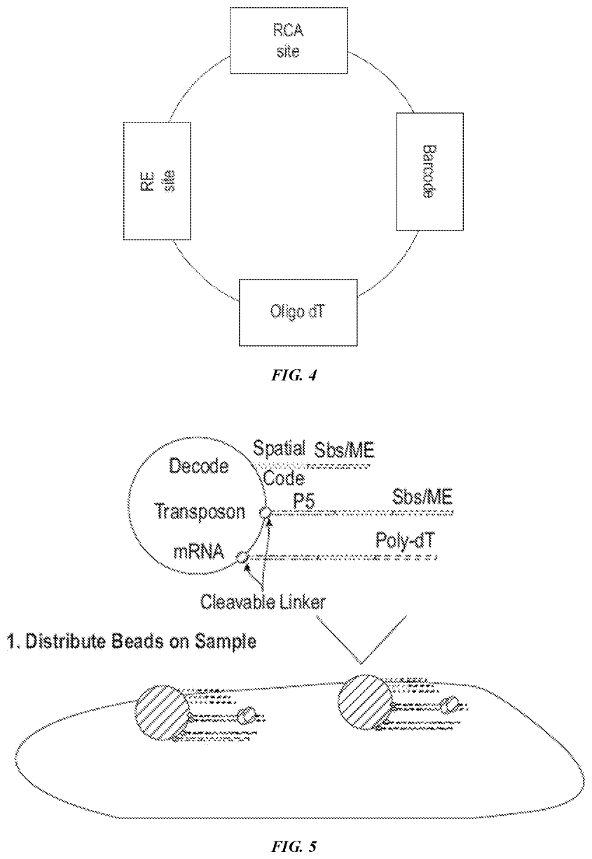

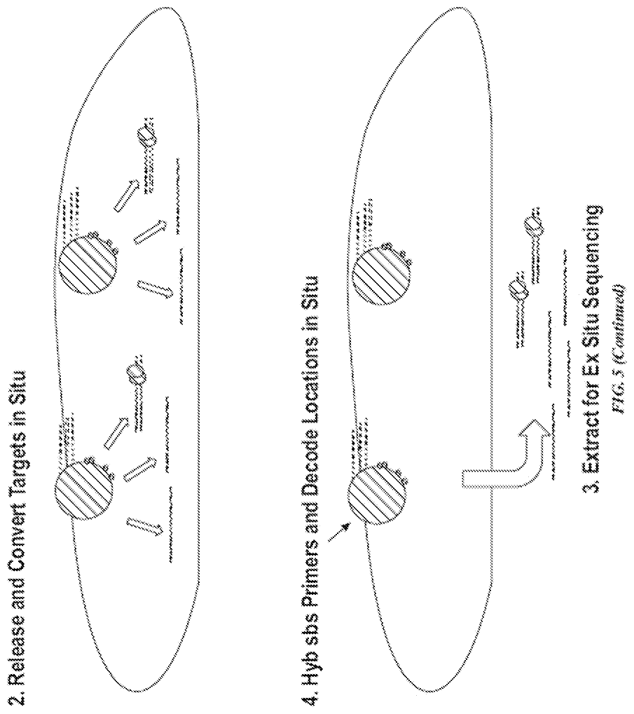

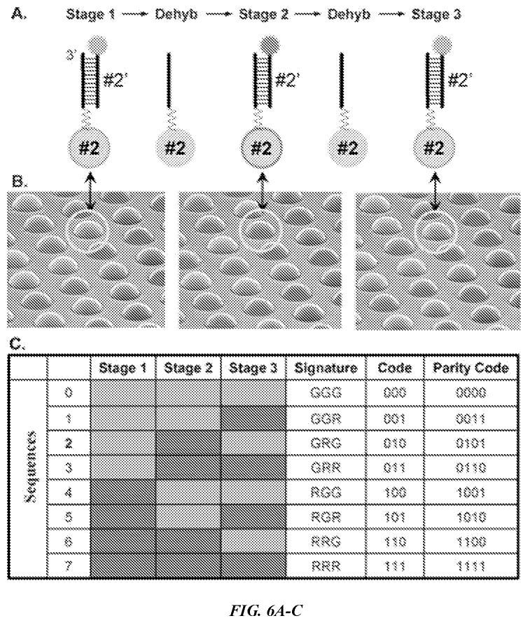

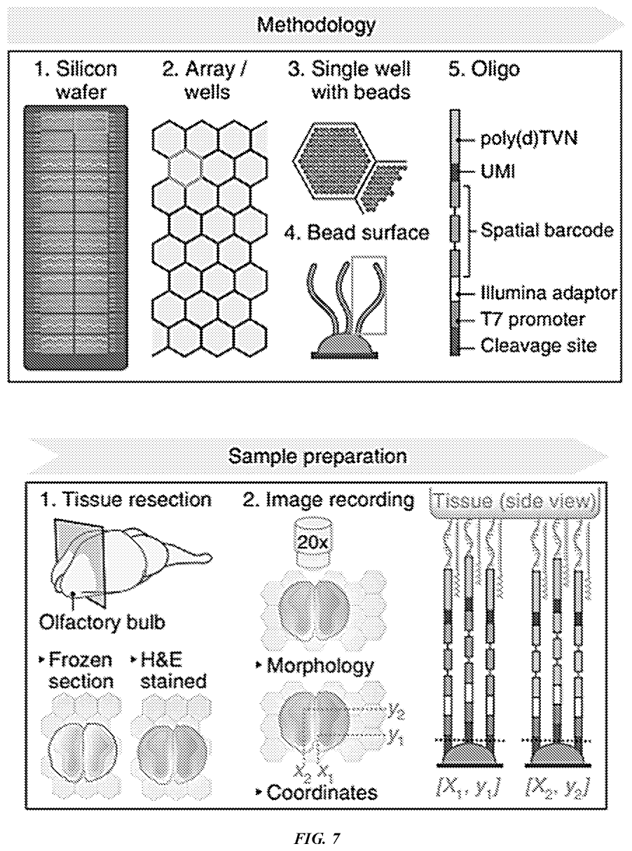

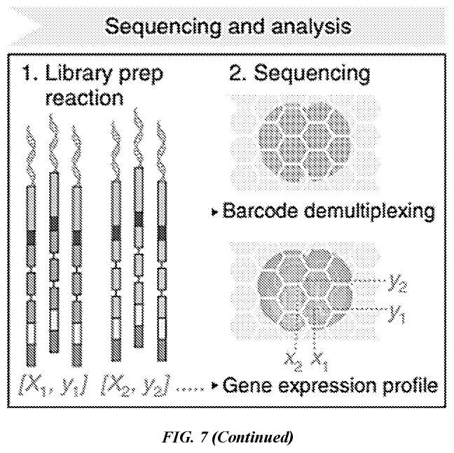

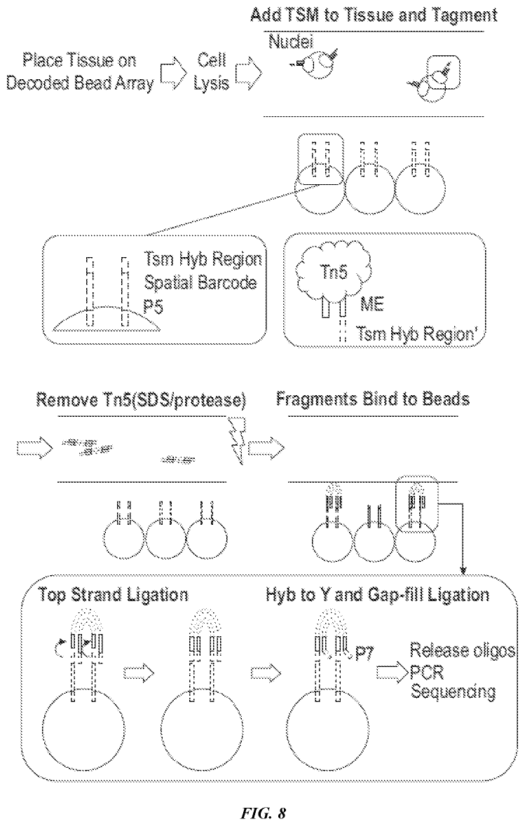

Work flow for in situ decoding of barcoded nanoparticles. FIG. 3 provides an exemplary workflow for in sit decoding of barcoded nanoparticles:
(1) Spatially barcoded capture bead/particles are generated. These particles could be dendrimers, clusterable particles, or gold nanoparticles or quantum dots that have been coated with oligos. Alternatively, the barcoded capture bead/particles can be a barcoded circular oligos that are amplified in tissue to generate a spatially barcoded nanoball (e.g., see FIG. 4).
(2) The spatially barcoded capture bead/particles are infused into tissue. For live tissue, particles can be functionalized with biological triggers of endocytosis and intracellular transport. They can also be targeted to specific subcellular organelles. In fixed or frozen tissues, particles can be perfused into the tissue after membrane permeabilization. Expansion techniques can also be used to expand the tissue to improve penetration by the nanoparticles. For spatially barcoded nanoballs, the protocol can be modified as follows: the tissue is incubated with LabelX to enable anchoring of RNA to a hydrogel; the tissue is expanded so as to allow better penetration by the spatially barcoded nanoballs by incubating the tissue in a hydrogel, digesting the protein and removing lipids, and expanding in ddH₂O; adding in circular barcodes and allow for diffusion through the tissue; performing rolling circle amplification and reversibly tethering nanoballs to hydrogel to prevent movement during sequencing (such as with photoactivated tethering and release).
(3) Tag captured material with barcode. mRNA can diffuse to the nanoparticle and hybridize to the oligos through the poly-dT tail. The oligo then acts as a primer for reverse transcription, and the resulting cDNA includes the spatial barcode. An alternative approach would be to cleave the oligos from the nanoparticle and allow them to diffuse into the tissue and encounter mRNA (e.g., see FIGS. 4 and 5). This could be advantageous in that the short oligos may diffuse more easily through the tissue compared to full-length mRNA. However, decoding of the nanoparticle would have to be performed before oligo release, or through non-cleavable decode oligos (e.g., see FIG. 5).
(4) Mapping of the spatial barcode within the tissue. There are at least three ways to map the spatial barcode within tissue: (a) direct in situ sequencing of the DNA barcoded oligos attached to the bread. (b) Use of multispectral beads (i.e., combinatorial lanthanide or Q-dot particles), where each color is associated with a specific DNA barcode sequence. For spatially barcoded nanoballs, the spatial barcodes are decoded in situ using the foregoing techniques. This may be difficult to implement as it would be challenging to generate enough unique colors to cover the desired barcode space. For example, lanthanides have been shown to produce 10\hat{ }3 different bead colors, whereas 10\hat{ }14 100 nm spheres can pack into a 5 mm cube. (c) Alternatively, a hybridization-based decoding scheme could be employed. In this scheme, numbers “tango” hybridization sites are encoded onto an oligo, and multiple rounds of hybridization with fluorescent complementary oligos of different colors occurs. It is possible to decode millions of unique combinations using this methodology with just three 15-mers, and this could be potentially be scaled to higher numbers. FIG. 6 outlines the decoding scheme. Separately, the oligo primer to which the mRNA is copied would have a barcode that is associated via a database with the tango codes. In this manner, when the cDNA is ultimately sequenced, the oligo barcode will be revealed, permitting determination of the spatial location of the bead. Optical z-sectioning can be performed on a sequencer with the sample on a flowcell, but another imaging platform, such as a light sheet microscope can also be leveraged. Another consideration is that the smaller the beads/particles (or in the limit that barcodes are comprised of a single oligo), the lower the signal will be during the sequencing/decoding step. In these cases, use of stochastic optical reconstruction microscopy (STORM), or related technologies can be utilized.
(5) Library preparation and sequencing. After reverse transcription, the tissue can be digested with protease and the cDNA molecules are extracted from the tissue after cleavage from the nanoparticles. Subsequent library prep steps can be performed in the tube, followed by sequencing. For spatially barcoded nanoballs, the nanoballs are chopped up with restriction enzymes that cut single-stranded DNA, like HaeIII, to release spatially barcoded oligos. mRNA then binds to the oligo-d(T) sequence, followed by cDNA synthesis.

Spatial transcriptomic assays that utilize oligonucleotide release from substrates to enhance the capture of RNA molecules. Current workflows using a barcoded surface suffer from low sensitivity and many RNA molecules are not detected in the final sequencing library. This may be partially due to inefficient capture of the RNA molecules in the initial steps of the protocol. In the original implementation, RNA molecules must diffuse from the tissue onto the bead array in order to hybridize with the capture oligos. Reversing this process (i.e., allowing the oligos to diffuse from the bead array into the tissue) may improve capture efficiency as the barcoded oligos are much shorter than the average mRNA transcript and therefore may diffuse more efficiently into the tissue (150 bp vs 3 kb; see FIG. 12A). The dual desthiobiotin linkage on the beads allows oligo release in a spatially controlled manner. Some lateral diffusion may still occur, but further experimental work would be needed to determine the extent of lateral diffusion with this method vs the standard method, as well as to ascertain if sensitivity is improved.

Applying an electric field with the positive electrode above the tissue may also help drive the oligos deep into the tissue (e.g., see FIG. 12B). However, the electric field will also cause the mRNAs to migrate; in theory, the shorter oligos should be able to catch up to the longer mRNA molecules, but shorter mRNA fragments may not be captured. An alternative approach would be to confer a positive charge to the oligos so that application of an electric field below the beads would result in the mRNA migrating towards the beads whereas the positively-charged spatial oligos would migrate towards the tissue (e.g., see FIG. 12C). Conferring a positive charge to the oligos could be accomplished by using a positively-charged peptide nucleic acid (PNA) oligo using the methods described in Ishizuka et al. (Nucleic Acid Research 36(5):1464-1471 (2008)); or an oligo attached to a positively charged gold nanoparticle using the methods described in Yao et al. (NPG Asia Mater 7:e159 (2015)) and Ojea-Jiménez et al. (ACS Nano 6(9):7692-7702 (2012)). These oligos could be hybridized to the spatially barcoded oligo at a site distal to the oligo(dT) sequence to prevent inhibition of reverse transcription, and then removed at a later step. Another option would be to synthesize the spatial barcoded oligos as a PNA/DNA hybrid oligo. Another possible concern is lateral diffusion of the fragments prior to capture by the barcoded oligonucleotides, which would reduce the spatial resolution of the assay. One determinant of the extent of lateral diffusion is the temperature; higher temperatures lead to greater diffusion. Current spatial transcriptomics workflows (e.g., 10× Visium, HDST) attain temperatures as high as 56° C. during the reverse transcription or permeabilization steps, which matches the highest temperature in the workflow prior to fragment capture (55° C. during tagmentation). Although experimental work will be required to assess the extent of lateral diffusion, the temperatures appear to be compatible with this type of workflow.

From the foregoing description, it will be apparent that variations and modifications can be made to the invention described herein to adopt it to various usages and conditions. Such embodiments are also within the scope of the following claims.

What is claimed:

1. A spatial genomics Assay for Transposase-Accessible Chromatin with high-throughput sequencing (ATAC-Seq) method, comprising
(A) loading nanoparticles into nanowells of an array, wherein the nanoparticles comprise oligos that attached to the nanoparticles through two desthiobiotin molecules (ddBio), the oligos comprising a P5 sequence, a spatial address sequence, and a transposome hybridization region;
(B) decoding the spatial address sequences of the oligos to determine the x,y position of the nanoparticles;
(C) placing tissue on top of the nanoparticles in the array, and lysing the cell membranes to access chromatin regions in the tissue;
(D) tagmentating the chromatin regions with a transposome complex to form tagmented fragments, and removing the transposome complex;
(E) permeabilizing the tissue to allow diffusion of the tagmented fragments to the nanoparticles in the array;
(F) capturing the tagmented fragments to the nanoparticles by hybridizing the tagmented fragments to the transposome hybridization region of the oligos;
(G) processing the captured tagmented fragments to make nanoparticle bound genomic library constructs for sequencing;
(H) releasing the genomic library constructs from the nanoparticles by using heat and added biotin;
(I) obtaining spatial genomics information of the tissue by sequencing the genomic library constructs using a sequencer and mapping the sequencing reads with x,y positions of the beads.

2. The spatial genomics ATAC-Seq method of claim 1, wherein the nanoparticles are beads.

3. The spatial genomics ATAC-Seq method of claim 1 or claim 2, wherein the transposome complex is a TN5 transposome complex.

4. The spatial genomics ATAC-Seq method of any one of the preceding claims, wherein the spatial address sequences are decoded by using a decoding-by-hybridization method.

5. The spatial genomics ATAC-Seq method of any one of the preceding claims, wherein the cell membranes are lysed by using a mild detergent.

6. The spatial genomics ATAC-Seq method of any one of the preceding claims, wherein the tissue is permeabilized by using a detergent and Proteinase K.

7. The spatial genomics ATAC-Seq method of any one of the preceding claims, where the captured tagmented fragments are processed by using top strand ligation, hybridization to Y-shaped adapters, and gap-filling ligations steps.

8. The spatial genomics ATAC-Seq method of any one of the preceding claims, wherein prior to sequencing the library constructs are amplified using PCR.

9. The spatial genomics ATAC-Seq method of any one of the preceding claims, wherein the sequencer utilizes sequencing by synthesis technology.

10. The spatial genomics ATAC-Seq method of any one of the preceding claims, wherein the hybridization region of the transposome complex is initially blocked by use of an accessory oligo that is then removed in step (E).

11. A spatial multi-omics Assay for Transposase-Accessible Chromatin with high-throughput sequencing (ATAC-Seq) and RNA-Seq method, comprising:
(A) loading nanoparticles into nanowells of an array, wherein the nanoparticles comprise two sets of oligos that attached to the nanoparticles through two desthiobiotin molecules (ddBio), the first set of oligos comprising a P5 sequence, a spatial address sequence, and a transposome hybridization region, the second set of oligos comprising a P5 sequence, a R1 sequencing primer site sequence, a spatial address sequence, a unique molecular identifier (UMI) sequence, and an oligo(dT) sequence;
(B) decoding the spatial address sequences of the oligos to determine the x,y position of the nanoparticles;
(C) placing tissue on top of the nanoparticles in the array, and lysing the cell membranes to access poly-A RNA transcripts and chromatin regions in the tissue;
(D) tagmentating the chromatin regions with a first transposome complex to from tagmented fragments, and removing the first transposome complex;
(E) permeabilizing the tissue to allow diffusion of the tagmented fragments and poly-A RNA transcripts to the nanoparticles in the array;
(F) capturing the tagmented fragments and poly-A RNA transcripts to the nanoparticles by (i) hybridizing the tagmented fragments to the transposome hybridization region of the first set of oligos, and (ii) hybridizing the poly-A RNA transcripts to the oligo(dT) sequence of the second set of oligos;
(G′) processing the first set of oligos comprising captured tagmented fragments to make nanoparticle bound genomic library constructs for sequencing;
(G″) reverse transcribing the second set of oligos comprising captured poly-A RNA transcripts in the presence of a single stranded template switch oligo to make nanoparticle bound cDNA library constructs for sequencing;
(H) releasing the genomic library constructs and the cDNA library constructs from the nanoparticles by using heat and added biotin;
(I) amplifying the genomic library constructs and cDNA library constructs using PCR and splitting the amplified products into two portions comprising both amplified constructs;
(J) amplifying a first portion of the amplified constructs with a P5 primer and a P7-i7-R2 primer to form an ATAC-Seq library;
(J′) tagmentating a second portion of the amplified constructs with R2-TSM and then amplifying the tagmented amplified library constructs using PCR with a P5-R1 primer and a P7-i7-R2 primer to form an RNA-Seq library; and
(K) obtaining multi-omics information by sequencing the ATAC-Seq library and RNA-Seq library using a sequencer and mapping the sequencing reads with x,y positions of the nanoparticles.

12. The spatial multi-omics ATAC-Seq and RNA-Seq method of claim 11, wherein the nanoparticles are beads.

13. The spatial multi-omics ATAC-Seq and RNA-Seq method of claim 11 or claim 12, wherein the transposome complex is a TN5 transposome complex.

14. The spatial multi-omics ATAC-Seq and RNA-Seq method of any one of claims 11 to 13, wherein the spatial address sequences are decoded by using a decoding-by-hybridization method.

15. The spatial multi-omics ATAC-Seq and RNA-Seq method of any one of claims 11 to 14, wherein the cell membranes are lysed by using a mild detergent.

16. The spatial multi-omics ATAC-Seq and RNA-Seq method of any one of claims 11 to 15, wherein the tissue is permeabilized by using a detergent and Proteinase K.

17. The spatial multi-omics ATAC-Seq and RNA-Seq method of any one of claims 11 to 16, where the captured tagmented fragments are processed by using top strand ligation, hybridization to Y-shaped adaptors, and gap-filling ligations steps.

18. The spatial multi-omics ATAC-Seq and RNA-Seq method of any one of claims 11 to 17, wherein prior to sequencing the library constructs are amplified using PCR.

19. The spatial multi-omics ATAC-Seq and RNA-Seq method of any one of claims 11 to 18, wherein the sequencer utilizes sequencing by synthesis technology.

20. The spatial multi-omics ATAC-Seq and RNA-Seq method of any one of claims 11 to 19, wherein the hybridization region of the transposome complex is initially blocked by use of an accessory oligo that is then removed in step (E).

21. The spatial multi-omics ATAC-Seq and RNA-Seq method of any one of claims 11 to 20, wherein steps (G) and (G′) are performed simultaneously.

22. The spatial multi-omics ATAC-Seq and RNA-Seq method of any one of claims 11 to 21, wherein steps (J) and (J′) are performed sequentially or concurrently.

23. The spatial multi-omics ATAC-Seq and RNA-Seq method of any one of claims 11 to 22, wherein the oligo d(T) sequence comprises 16 to 20 nucleotides.

24. A spatial transcriptomics method using a beadchip, comprising:
(A) loading beads into nanowells of a beadchip, wherein the beads comprise oligos comprising a P5 or a P7 sequence, a spatial address sequence, an optional sequence primer site, and an oligo(dT) sequence;
(B) decoding the spatial address sequences of the oligos to determine the x,y position of the beads;
(C) placing tissue into hyb-seal sectioned areas of the beadchip, and lysing the cell membranes to access poly-A RNA transcripts in the tissue;
(D) capturing the poly-A RNA transcripts to the beads;
(E) reverse transcribing the oligos comprising captured poly-A RNA transcripts: (i) in the presence of a single stranded template switch oligo to make a bead bound cDNA library constructs for sequencing, or (ii) further performing a single strand ligation reaction with a single stranded oligo to make a bead bound cDNA library constructs for sequencing;
(F) amplifying from the hyb-seal section the bead bound cDNA library constructs using PCR with a P5 primer or a P7 primer; and
(G) obtaining transcriptomics information by sequencing the amplified cDNA library constructs using a sequencer and mapping the sequencing reads with x,y positions of the beads in the beadchip.

25. The spatial transcriptomics method of claim 24, wherein the beadchip has a feature density (feature/mm²) of greater than 300000.

26. The spatial transcriptomics method of claim 25, wherein the beads comprise lanthanide nanophosphor labels.

27. A spatial multi-omics method to detect targeted RNA and proteins, comprising:
(A) loading multiple sets of lanthanide nanophosphor labeled beads into an array, wherein a first set of beads comprise oligos comprising a P5 adapter sequence or a P7 adapter sequence, a spatial address sequence, an optional sequence primer site, and an oligo(dT) sequence, and a second set of beads that comprise oligos comprising a spatial address sequence, a UMI sequence, and a capture sequence;
(B) placing tissue on top of the beads, and lysing the cell membranes to access poly-A RNA transcripts and proteins in the tissue;
(C) adding antibodies comprising a barcode nucleotide sequence which bind with specificity to protein targets;
(D) capturing the poly-A RNA transcripts to the first set of beads;
(D′) capturing the antibodies to the second set of beads by hybridizing the barcode nucleotide sequence of the antibodies to the capture sequence;
(E) reverse transcribing the oligos comprising captured poly-A RNA transcripts with oNTPS;
(F) adding a primer which binds to oligos on the sets of beads and extending the primers with oNTPS;
(G) adding a dsDNA dye that binds to the extended primer sequences; and
(H) detecting targeted RNA and proteins by imaging and decoding the sets of beads.

28. The spatial multi-omics method of claim 27, wherein the dsDNA dye is PicoGreen.

29. The spatial multi-omics method of claim 27 or claim 28, wherein the imaging and decoding the sets of beads is performed simultaneously.

30. The spatial multi-omics method of any one of claims 27 to 29, wherein steps (D) and (D′) are performed simultaneously.

31. A method for in situ decoding of spatially addressed nano-particles, comprising:
(A) infusing nanoparticles comprising immobilized oligos into tissue, the oligos comprising a P5 sequence, a R1 sequence primer site, a spatial address sequence, and an oligo d(T) sequence;
(B) capturing mRNA in situ by hybridizing mRNA in the tissue with the oligo d(T) sequence of the oligos;
(C) reverse transcribing the oligos comprising captured poly-A RNA transcripts in situ to form a nanoparticle bound cDNA construct;
(D) mapping the cDNA construct in situ by:
(i) direct in situ sequencing of the spatial address sequence; or
(ii) using decoding-by-hybridization approach to decode the spatial address sequence in situ; and
(E) digesting the tissue with a detergent and Proteinase K, and isolating the tagged constructs;
(F) separating the cDNA construct from the nanoparticle, library prepping the cDNA construct and sequencing the cDNA construct.

32. A method for in situ decoding of spatially addressed nano-particles, comprising:
(A) infusing nanoparticles comprising multiple sets of immobilized oligos into a tissue, the multiple sets of immobilized oligos comprising:
a first set of oligos comprising an adapter sequence, a sequence primer site, a spatial address sequence, and an oligo d(T) sequence,
a second set of oligos comprising a spatial address sequence and a Sbs/ME sequence,
an optional third set of oligos comprising the adapter sequence, a spatial address sequence, and a Sbs/ME sequence,
wherein the first and third set of oligos are attached to the nanoparticles by a cleavable linker, and wherein the second set of oligos are not attached to the nanoparticles by a cleavable linker;
(B) capturing mRNA in situ by hybridizing mRNA in the tissue with the oligo d(T) sequence of the first set of oligos;
(C) reverse transcribing the oligos comprising captured poly-A RNA transcripts in situ to form a nanoparticle bound cDNA construct;
(D) extracting ex situ the cDNA construct from the nanoparticle by cleaving the cleavable linker and separating the cDNA construct from the nanoparticle;
(E) mapping the second set of oligos in situ by:
(i) direct in situ sequencing of the spatial address sequence; or
(ii) using decoding-by-hybridization approach to decode the spatial address sequence in situ; and
(F) library prepping the cDNA construct and sequencing the cDNA construct.

33. The method of claim 31 or claim 32, wherein the oligo-coated nanoparticles are functionalized with biological triggers for endocytosis and intracellular transport, or with targeting moieties or ligands for specific subcellular organelles.

34. The method of any one of claims 31 to 33, wherein the oligo-coated nanoparticles further comprise lanthanide nanophosphor labels or Q-dot particles.

35. A spatial genomics method using ex situ spatial capture on a beadchip, comprising:
(A) loading beads into nanowells of a beadchip, wherein the beads comprise oligos that attached to the nanoparticles through biotin molecule(s), the oligos comprising a P5 sequence, a spatial address sequence, and a first capture sequence;
(B) decoding the spatial address sequences of the oligos to determine the x,y position of the beads;
(C) placing tissue into sectioned areas of the beadchip, lysing the cell membranes, and adding non-tethered pool of oligos comprising a P7 sequence, an optional UMI sequence, a sample index sequence, and a second capture sequence, wherein the first and second capture sequence bind to different portions of a biomolecule;
(D) capturing the biomolecule to the first and second capture sequence of the oligos;
(E) extending and/or ligating the first capture sequence to the second capture sequence to form a construct which comprises the P5 sequence, the spatial address sequence, the first capture sequence, a gap or bridge molecule of interest, the second capture sequence, the optional UMI sequence, and the P7 sequence; and
(F) obtaining ex situ spatial genomics information of the tissue by sequencing the construct using a sequencer and mapping the sequencing reads with x,y positions of the beads.

36. The method of claim 35, wherein the first capture sequence and the second capture sequence having complementary sequences to a targeted gene.

37. The method of claim 35 or claim 36, wherein the gap or bridge molecule of interest comprise nucleotides up to a thousand nucleotides in length.

38. A spatial genomics Assay for Transposase-Accessible Chromatin with high-throughput sequencing (ATAC-Seq) method, comprising:
(A) providing a substrate which comprises oligos attached to the substrate through a selectively cleavable linker, the oligos comprising an adapter sequence, a spatial address sequence, and a transposome hybridization region, wherein if the substrate is an ordered substrate, then the spatial address sequence of the oligos is optional;
(B) decoding the spatial address sequences of the oligos to determine the x,y positions of the oligos on the substrate, wherein if the substrate is an ordered substrate, then step (B) is optional;
(C) placing tissue on top of the substrate, and lysing the cell membranes to access chromatin regions in the tissue;
(D) tagmentating the chromatin regions with a transposome complex to form tagmented fragments, and removing the transposome complex;
(E) permeabilizing the tissue to allow diffusion of the tagmented fragments to the substrate;
(F) capturing the tagmented fragments to the substrate by hybridizing the tagmented fragments to the transposome hybridization region of the oligos;
(G) processing the captured tagmented fragments to make substrate bound genomic library constructs for sequencing;
(H) releasing the genomic library constructs from the substrate by selectively cleaving the linker;
(I) obtaining spatial genomics information of the tissue by sequencing the genomic library constructs using a sequencer and mapping the sequencing reads with x,y positions of the oligos.

39. The spatial genomics ATAC-Seq method of claim 38, wherein the transposome complex is a TN5 transposome complex.

40. The spatial genomics ATAC-Seq method of claim 38 or claim 39, wherein the substrate is a plate, a multiwell plate, a slide, a flowcell, or nanoparticles, optionally, wherein at least a portion of the substrate comprises a streptavidin coating.

41. The spatial genomics ATAC-Seq method of any one of claims 38 to 40, wherein the substrate comprises areas of immobilized oligos separated by interstitial areas lacking immobilized oligos.

42. The spatial genomics ATAC-Seq method of claim 41, wherein the substrate comprises islands or clusters of immobilized oligos separated by interstitial areas lacking immobilized oligos, and wherein each island or cluster of immobilized oligos has a spatial address sequence that is different from the spatial address sequence of the other islands or clusters of immobilized oligos.

43. The spatial genomics ATAC-Seq method of any one of claims 38 to 42, wherein the spatial address sequences are decoded by using a decoding-by-hybridization method.

44. The spatial genomics ATAC-Seq method of any one of claims 38 to 41, wherein the substrate is an ordered substrate and the x,y positions of the oligos on the substrate are predetermined or can be readily determined without having to decode the spatial address sequences in step (B).

45. The spatial genomics ATAC-Seq method of any one of claims 38 to 44, wherein the cell membranes are lysed by using a mild detergent.

46. The spatial genomics ATAC-Seq method of any one of claims 38 to 45, wherein the tissue is permeabilized by using a detergent and Proteinase K.

47. The spatial genomics ATAC-Seq method of any one of claims 38 to 46, wherein the captured tagmented fragments are processed by using top strand ligation, hybridization to Y-shaped adaptors, and gap-filling ligations steps.

48. The spatial genomics ATAC-Seq method of any one of claims 38 to 47, wherein prior to sequencing the library constructs are amplified using PCR.

49. The spatial genomics ATAC-Seq method of any one of claims 38 to 48, wherein the sequencer utilizes sequencing-by-synthesis technology.

50. The spatial genomics ATAC-Seq method of any one of claims 38 to 49, wherein the hybridization region of the transposome complex is initially blocked by use of an accessory oligo that is then removed in step (E).

51. A spatial multi-omics Assay for Transposase-Accessible Chromatin with high-throughput sequencing (ATAC-Seq) and RNA-Seq method, comprising:
(A) providing a substrate which comprises multiple sets of immobilized oligos attached to the substrate through selectively cleavable linker(s), wherein the substrate comprises a first set of oligos which comprises a first adapter sequence, a spatial address sequence, and a transposome complex hybridization region, and a second set of oligos that comprises a second adapter sequence, a sequencing primer site sequence, a spatial address sequence, a unique molecular identifier (UMI) sequence, and an oligo(dT) sequence, wherein if the substrate is an ordered substrate, then the spatial address sequence of the first set of oligos, and/or second set of oligos are optional;
(B) decoding the spatial address sequences of the oligos to determine the x,y position of the oligos on the substrate, wherein if the substrate is an ordered substrate, then step (B) is optional;
(C) placing tissue on top of the substrate, and lysing the cell membranes to access poly-A RNA transcripts and chromatin regions in the tissue;
(D) tagmentating the chromatin regions with a first transposome complex to form tagmented fragments, and removing the first transposome complex;
(E) permeabilizing the tissue to allow diffusion of the tagmented fragments and poly-A RNA transcripts to the substrate;
(F) capturing the tagmented fragments and poly-A RNA transcripts to the substrate by (i) hybridizing the tagmented fragments to the transposome hybridization region of the first set of oligos, and (ii) hybridizing the poly-A RNA transcripts to the oligo(dT) sequence of the second set of oligos;
(G′) processing the first set of oligos comprising captured tagmented fragments to make substrate bound genomic library constructs for sequencing;
(G″) reverse transcribing the second set of oligos comprising captured poly-A RNA transcripts in the presence of a single stranded template switch oligo to make substrate bound cDNA library constructs for sequencing;
(H) releasing the genomic library constructs and the cDNA library constructs from the substrate by selectively cleaving the linker(s);
(I) amplifying the genomic library constructs and cDNA library constructs using PCR and splitting the amplified products into two portions comprising both amplified constructs;
(J) amplifying a first portion of the amplified constructs with a first adapter primer and a second adapter primer to form an ATAC-Seq library, wherein the first adapter primer and the second adapter primer bind to first adapter sequence and second adapter sequence, respectively, and wherein the first adapter primer and/or the second adapter primer further comprises an index sequence and/or a sequencing primer site sequence;
(J′) tagmentating a second portion of the amplified constructs with a primer comprising the sequence primer sequence and a transposome complex (TSM) and then amplifying the tagmented amplified library constructs using PCR with the first adapter primer comprising a sequencing primer site sequence and the second adapter primer comprising an index sequence and a sequencing primer site sequence to form an RNA-Seq library, wherein the first adapter primer and the second adapter primer have difference sequence primer sequences; and
(K) obtaining multi-omics information by sequencing the ATAC-Seq library and RNA-Seq library using a sequencer and mapping the sequencing reads with x,y positions of the oligos.

52. The spatial multi-omics ATAC-Seq and RNA-Seq method of claim 51, wherein the transposome complex is a TN5 transposome complex.

53. The spatial multi-omics ATAC-Seq and RNA-Seq method of claim 51 or claim 52, wherein the substrate is a plate, a multiwell plate, a slide, a flowcell, or nanoparticles, optionally, wherein at least a portion of the substrate comprises a streptavidin coating.

54. The spatial genomics ATAC-Seq method of any one of claims 51 to 53, wherein the substrate comprises areas of immobilized oligos separated by interstitial areas lacking immobilized oligos.

55. The spatial multi-omics ATAC-Seq and RNA-Seq method of claim 54, wherein the substrate comprises islands or clusters of the two sets of immobilized oligos separated by interstitial areas lacking immobilized oligos, and wherein each island or cluster of the immobilized oligos has a spatial address sequence that is different from the spatial address sequence of the other islands or clusters of immobilized oligos.

56. The spatial multi-omics ATAC-Seq and RNA-Seq method of any one of claims 51 to 55, wherein the spatial address sequences are decoded by using a decoding-by-hybridization method.

57. The spatial multi-omics ATAC-Seq and RNA-Seq method of claim 51 or claim 52, wherein the substrate is an ordered substrate and the x,y positions of the oligos on the substrate are predetermined or can be readily determined without having to decode the spatial address sequences in step (B).

58. The spatial multi-omics ATAC-Seq and RNA-Seq method of any one of claims 51 to 57, wherein the cell membranes are lysed by using a mild detergent.

59. The spatial multi-omics ATAC-Seq and RNA-Seq method of any one of claims 51 to 58, wherein the tissue is permeabilized by using a detergent and Proteinase K.

60. The spatial multi-omics ATAC-Seq and RNA-Seq method of any one of claims 51 to 59, where the captured tagmented fragments are processed by using top strand ligation, hybridization to Y-shaped adaptors, and gap-filling ligations steps.

61. The spatial multi-omics ATAC-Seq and RNA-Seq method of any one of claims 51 to 60, wherein prior to sequencing the library constructs are amplified using PCR.

62. The spatial multi-omics ATAC-Seq and RNA-Seq method of any one of claims 51 to 61, wherein the sequencer utilizes sequencing-by-synthesis technology.

63. The spatial multi-omics ATAC-Seq and RNA-Seq method of any one of claims 51 to 62, wherein the hybridization region of the transposome complex is initially blocked by use of an accessory oligo that is then removed in step (E).

64. The spatial multi-omics ATAC-Seq and RNA-Seq method of any one of claims 51 to 63, wherein steps (G) and (G′) are performed sequentially or concurrently.

65. The spatial multi-omics ATAC-Seq and RNA-Seq method of any one of claims 51 to 64, wherein steps (J) and (J′) are performed sequentially or concurrently.

66. The spatial multi-omics ATAC-Seq and RNA-Seq method of any one of claims 51 to 65, wherein the oligo d(T) sequence comprises 16 to 20 nucleotides.

67. A spatial transcriptomics method, comprising:
(A) providing a substrate comprising features, wherein oligos are immobilized on the features of the substrate, wherein the oligos comprise an adapter sequence, a spatial address sequence, an optional sequence primer site, and an oligo(dT) sequence, wherein if the substrate is an ordered substrate, then the spatial address sequence of the oligos is optional;
(B) decoding the spatial address sequences of the oligos to determine their x,y position in the substrate, wherein if the substrate is an ordered substrate, then step (B) is optional;
(C) placing tissue onto the substrate, and lysing the cell membranes to access poly-A RNA transcripts in the tissue;
(D) capturing the poly-A RNA transcripts to the oligos;
(E) reverse transcribing the oligos comprising captured poly-A RNA transcripts: (i) in the presence of a single stranded template switch oligo to make substrate bound cDNA library constructs for sequencing, or (ii) further performing a single strand ligation reaction with a single stranded oligo to make substrate bound cDNA library constructs for sequencing;
(F) amplifying the oligo bound cDNA library constructs using PCR with an adapter primer;
(G) obtaining transcriptomics information by sequencing the amplified cDNA library constructs using a sequencer and mapping the sequencing reads with x,y positions of the oligos on the features of the substrate.

68. The spatial transcriptomics method of claim 67, wherein the substrate is a microarray, a plate, a multiwell plate, or a flowcell.

69. The spatial multi-omics ATAC-Seq and RNA-Seq method of claim 67 or claim 68, wherein the substrate is an ordered substrate and the x,y positions of the oligos on the features of the substrate are predetermined, or can be readily determined without having to decode the spatial address sequences in step (B).

70. The spatial transcriptomics method of any of claims 67 to 69, wherein the substrate has a feature density (feature/mm²) of greater than 300000.

71. The spatial transcriptomics method of any one of claims 67 to 70, wherein the features of the substrate and/or oligos further comprise lanthanide nanophosphor labels.

72. A spatial multi-omics method to detect targeted RNA and proteins, comprising:
(A) providing a substrate comprising features having lanthanide nanophosphor labels, wherein the features comprise multiple sets of immobilized oligos, wherein a first set of oligos comprise an adapter sequence, a spatial address sequence, an optional sequence primer site, and an oligo(dT) sequence, and a second set oligos that comprise a spatial address sequence, a UMI sequence, and a capture sequence, and wherein if the substrate is an ordered substrate, then the spatial address sequence of the first set of oligos and/or the second set of oligos are optional;
(B) decoding the spatial address sequences of the oligos to determine their x,y position in the substrate, wherein if the substrate is an ordered substrate, then step (B) is optional;
(C) placing tissue on top of the substrate, and lysing the cell membranes to access poly-A RNA transcripts and proteins in the tissue;
(D) adding antibodies comprising a barcode nucleotide sequence which bind with specificity to a targeted protein(s);
(E) capturing the poly-A RNA transcripts to the first set of oligos;
(E′) capturing the antibodies to the second set of oligos by hybridizing the barcode nucleotide sequence of the antibodies to the capture sequence;
(F) reverse transcribing the oligos comprising captured poly-A RNA transcripts with oNTPS;
(G) adding a primer which binds to oligos on the features of the substrate and extending the primers with oNTPS;
(H) adding a dsDNA dye that binds to the extended primer sequences; and
(I) detecting targeted RNA and proteins by imaging, and determining the x,y positions of the first and second sets of oligos on the features of the substrate.

73. The spatial multi-omics method of claim 72, wherein the substrate is a microarray, a plate, a multiwell plate, or a flowcell.

74. The spatial multi-omics method of claim 72 or claim 73, wherein the substrate is an ordered substrate and the x,y positions of the oligos on the features of the substrate are predetermined, or can be readily determined without having to decode the spatial address sequences in step (B).

75. The spatial multi-omics method of any one of claims 72 to 74, wherein the substrate has a feature density (feature/mm²) of greater than 300000.

76. The spatial multi-omics method of any one of claims 72 to 75, wherein the dsDNA dye is a cyanine-based dye.

77. The spatial multi-omics method of claim 76, wherein the cyanine-based dye is selected from SYBR Green I, PicoGreen, SYBR Safe, SYBR Gold, thiazole orange, oxazole yellow, Safe-Green and Chai Green.

78. The spatial multi-omics method of any one of claims 72 to 77, wherein steps (E) and (E′) are performed sequentially or concurrently.

79. A method for in situ decoding of spatially addressed oligos, comprising:
(A) infusing an oligo-coated substrate into tissue, the oligo-coated substrate comprising immobilized oligos comprising an adapter sequence, a sequence primer site, a spatial address sequence, and an oligo d(T) sequence;
(B) capturing mRNA in situ by hybridizing mRNA in the tissue with the oligo d(T) sequence of the oligos;
(C) reverse transcribing the oligos that comprise captured poly-A RNA transcripts in situ to form a substrate bound cDNA construct;
(D) mapping the cDNA construct in situ by:
(i) direct in situ sequencing of the spatial address sequence; or
(ii) using decoding-by-hybridization approach to decode the spatial address sequence in situ; and
(E) digesting the tissue with a detergent and Proteinase K, and isolating the substrate bound cDNA construct; and
(F) separating the cDNA construct from the substrate, library prepping the cDNA construct and sequencing the cDNA construct.

80. The method of claim 79, wherein the substrate is from 10 nm to 10 μm in size.

81. The method of claim 79 or claim 80, wherein the substrate is functionalized with biological triggers for endocytosis and intracellular transport, or with targeting moieties or ligands for specific subcellular organelles.

82. The method of any one of claims 79 to 81, wherein the substrate or oligos comprise lanthanide nanophosphor labels or Q-dot particles.

83. A method for in situ decoding of spatially addressed oligos, comprising:
(A) infusing an oligo-coated substrate into tissue, the oligo-coated substrate comprising multiple sets of immobilized oligos sets, the multiple sets of immobilized oligos comprising:
a first set of oligos comprising a first adapter sequence, a sequence primer site, a spatial address sequence, and an oligo d(T) sequence,
a second set of oligos comprising a spatial address sequence and a Sbs/ME sequence,
an optional third set of oligos comprising the first adapter sequence, a spatial address sequence, and a Sbs/ME sequence,
wherein the first and third set of oligos are attached to the substrate by a selectively cleavable linker, and wherein the second set of oligos are not attached to the substrate by a selectively cleavable linker;
(B) capturing mRNA in situ by hybridizing mRNA in the tissue with the oligo d(T) sequence of the first set of oligos;
(C) reverse transcribing the oligos comprising captured poly-A RNA transcripts in situ to form a substrate bound cDNA construct;
(D) extracting ex situ the cDNA construct from the substrate by selectively cleaving the cleavable linker and separating the cDNA construct from the substrate;
(E) mapping the second set of oligos in situ by:
(i) direct in situ sequencing of the spatial address sequence; or
(ii) using decoding-by-hybridization approach to decode the spatial address sequence in situ; and
(F) library prepping the cDNA construct and sequencing the cDNA construct.

84. The method of claim 83, wherein the substrate is from 10 nm to 10 μm in size.

85. The method of claim 83 or claim 84, wherein the substrate is functionalized with biological triggers for endocytosis and intracellular transport, or with targeting moieties or ligands for specific subcellular organelles.

86. The method of any one of claims 83 to 85, wherein the substrate or multiple sets of oligos comprise lanthanide nanophosphor labels or Q-dot particles.

87. A spatial genomics method using ex situ spatial capture on a substrate, comprising:
(A) providing a substrate comprising features, wherein the features comprise immobilized oligos that attached to the features of the substrate through a selectively cleavable linker, the oligos comprising a first adapter sequence, a spatial address sequence, and a first capture sequence, wherein if the substrate is an ordered substrate, then the spatial address sequence of the oligos is optional;
(B) decoding the spatial address sequences of the oligos to determine the x,y position of the beads, wherein if the substrate is an ordered substrate, then step (B) is optional;
(C) placing tissue on the substrate, lysing the cell membranes, and adding non-tethered pool of oligos comprising a second adapter sequence, an optional UMI sequence, a sample index sequence, and a second capture sequence, wherein the first and second capture sequence bind to different portions of a biomolecule;
(D) capturing the biomolecule to the first and second capture sequence of the oligos;
(E) extending and/or ligating the first capture sequence to the second capture sequence to form a construct which comprises the first adapter sequence, the spatial address sequence, the first capture sequence, a gap or bridge molecule of interest, the second capture sequence, the optional UMI sequence, and the second adapter sequence; and
(F) obtaining ex situ spatial genomics information of the tissue by sequencing the construct using a sequencer and mapping the sequencing reads with the determined x,y positions of the oligos.

88. The spatial multi-omics method of claim 87, wherein the substrate is a microarray, a plate, a multiwell plate, or a flowcell.

89. The spatial multi-omics method of claim 87 or claim 88, wherein the substrate is an ordered substrate and the x,y positions of the oligos on the features of the substrate are predetermined, or can be readily determined without having to decode the spatial address sequences in step (B).

90. The spatial multi-omics method of any one of claims 87 to 89, wherein the substrate has a feature density (feature/mm²) of greater than 300000.

91. The method of any one of claims 87 to 90, wherein the first capture sequence and the second capture sequence having complementary sequences to a targeted gene.

92. The method of any one of claims 87 to 91, wherein the gap or bridge molecule of interest comprise nucleotides up to a thousand nucleotides in length.
